(19) United States
(12) Patent Application Publication
(10) Pub. No.: US 2022/0323390 A1
(43) Pub. Date: Oct. 13, 2022

(54) DIETARY SUPPLEMENT TO PROMOTE GUT HEALTH

(71) Applicant: TERRAGENICS NUTRITION, INC., Orem, UT (US)

(72) Inventor: Jennifer COOPER, Nephi, UT (US)

(21) Appl. No.: 17/640,084

(22) PCT Filed: Sep. 4, 2020
(86) PCT No.: PCT/US2020/049488

§ 371 Date: Mar. 3, 2022

Related U.S. Application Data
(60) Provisional application No. 62/897,156, filed on Sep. 6, 2019.
(60) Provisional application No. 62/897,630, filed on Sep. 9, 2019.

(51) Int. Cl. A61K 31/198; A61K 9/00; A61K 31/702; A61K 31/05; A61K 35/76; A61K 38/47; A61K 38/48; A61K 38/46; A61K 38/16; A61K 38/17; A61K 38/39; A61K 33/22; A61K 33/34; A61K 33/24; A61K 33/26; A61K 33/32; A61K 33/30; A61P 1/14

(52) U.S. Cl. CPC A61K 31/198; A61K 9/0053; A61K 31/702; A61K 31/05; A61K 35/76; A61K 38/47; A61K 38/48; A61K 38/465; A61K 38/4873; A61K 38/168; A61K 38/1767; A61K 38/39; A61K 38/1706; A61K 38/16; A61K 33/22; A61K 33/34; A61K 33/24; A61K 33/26; A61K 33/32; A61K 33/30; A61P 1/14

(57) ABSTRACT

Provided are dietary supplement composition comprising one or both of L-theanine and a trace mineral complex, optionally in combination with bioactive peptides, in an amount or amounts individually or in combination effective to promote gut health. Further optional components include a fiber oligosaccharide prebiotic, a polyphenol prebiotic, and a polynucleotide or blend. Examples of benefits include one or more of: —an increase in the amount of total short-chain fatty acid production or of at least one of propionate, acetate and butyrate; —an increase the expression or level of at least one tight junction protein (for example, claudin-1, claudin-3, claudin-4, occludin, or a zona occludens protein) or mucin-3 or decrease the expression or level of claudin-2; —an increase in transepithelial electrical resistance; —an increase in the abundance or diversity of a member or phylum of the intestinal bacterial community of the Intestinal lumen or mucus area (for example, Firmicutes, Bacteroidetes, Lactobacilli, Bifidobacteria, F. prausnitzii, or A. muciniphila); —an increase in the expression or level of at least one intestinal surface area marker protein (for example, villin, myosin, cadherin or sucrase-isomaltase). Methods for using such supplements are also disclosed.

### RELATED APPLICATIONS

The present application claims the priority of U.S. provisional applications No. 62/897,156 filed 6 Sep. 2019 and No. 62/897,630 filed 9 Sep. 2019. The disclosures of both prior applications are incorporated by reference in their entirety.

### FIELD OF THE DISCLOSURE

The present disclosure is directed to dietary supplements, i.e., compositions to promote healthy gut structure, with the aim of repairing or strengthening the gut barrier function and/or improving the environment of the lumen and/or balancing the gut microbiota, and to methods for their preparation and use of such dietary supplements. In another aspect, the present disclosure is directed to methods of promoting gut health, including specific aspects thereof (such as promoting integrity and thus improving barrier function of the intestinal barrier, increasing gut surface area for example by increasing one or more surface area indicators) in a subject by daily administering to the subject the dietary supplements of the present disclosure.

### BACKGROUND OF THE DISCLOSURE

The human intestine has been reported to have about 10¹⁴ bacterial cells/mi of luminal content, and the composition of the gut microflora has been found to play important roles in the health and diseases of humans. Gionchettu P, et al, Probiotics in infective diarrhea and inflammatory bowel diseases. J Gastroenterol 2000; 108: 975-82; Gill H S. Stimulation of the immune system. Int Dairy J 1998; 8: 535-44; Salminen S, et al. Functional food science and gastrointestinal physiology and function. Br J Nutr 1998; 80: S147-71. These estimates are being revised as estimating techniques and the accuracy of supporting data are improved. See, e.g., Sender R, Fuchs S, Milo R. Revised Estimates for the Number of Human and Bacteria Cells in the Body. PLoS Biol. 2016; 14(8):e1002533. Published 2016 Aug. 19. doi:10.1371/journal.pbio.1002533. This reference estimates the number of bacteria in the human body to be 3.8×10¹³.

The intestinal barrier is mainly formed by a layer of epithelial cells joined together by tight junctions (TJ). TJ consist mainly of the transmembrane protein complexes (e.g. claudins and occludins) and ZO (zonulae occludentes), a group of cytosolic proteins. These proteins form a structure at the boundary of two adjacent cells, working as a barrier within the epithelial cell space. The TJ proteins act as a rate-limiting barrier in the paracellular pathway in forming a selectively permeable barrier. Pro-inflammatory cytokines have been shown to down-regulate TJ protein expression. Al-Sadi R, et al, (2009) Mechanism of cytokine modulation of epithelial tight junction barrier. Front Biosci 14, 2765-2778. An increase in one or more claudins (except claudin-2 which should decrease) and occluding protein level or expression is associated with a healthier and more selective intestinal barrier. Id.

The intestinal barrier is a functional unit, organized as a multi-layer system, made up of two main components: a physical barrier surface, which prevents bacterial adhesion and regulates paracellular diffusion to the host tissues, and a deeper functional barrier, that is able to discriminate between pathogens and commensal microorganisms, organizing the immune tolerance and the immune response to pathogens.

The paracellular permeability of the intestine to ions and small molecules is dependent on the intestinal location and is controlled by the TJ protein complexes that connect adjacent cells in the epithelium. Inflammatory stimuli can increase permeability of the epithelium through contractions of the perijunctional actomyosin ring that is connected to the Ti complex or via altered TJ protein composition or dynamics. Chronic changes in epithelial permeability are considered to contribute to the pathophysiology of several intestinal disorders by allowing antigens or inflammatory stimuli including microorganisms to enter the lamina propria (LP) and perturb homeostasis. In obesity, for example, altered epithelial permeability and permeation of the gut by lipopolysaccharide (LPS) can lead to increased levels in the plasma and insulin-resistant states.

The proper functioning of the gut barrier has implications beyond the gut and mucosal immunity affecting metabolism, adaptive immunity, the enteric nervous system, and the brain. The intestinal barrier function plays a central role in how gut-brain interaction affects the immune system. Deterioration of the intestinal barrier function may lead to increased and prolonged mucosal immune activation and, consequently, to increased afferent sensory signaling and abdominal complaints. Brain function affects the intestinal barrier partly by activation of the HPA-axis and both mast cell-dependent as well as mast cell-independent mechanisms. Traina, G. Frontiers in Cellular Neuroscience 1 Jul. 2019 | Volume 13 | Article 345. Furthermore, the role of microbial metabolites such as butyrate have gained Interest in this respect. (Wells, J M et al, Am J Physiol Gastrointest Liver Physiol. 2017 Mar. 1; 312(3): G171-G193. Published online 2016 Dec. 1. doi: 10.1152/ajpgi.00048.2015). Accordingly, there is a need to maintain or restore the integrity of the intestinal barrier in order to strengthen the defenses of the host against invading organisms and to keep the host's immune system activity in balance.

The human gut microbiota is a complex ecosystem that includes at least 10¹⁴ bacteria, counting up to 2000 species, with predominance of anaerobic bacteria as well as other microorganisms including bacteriophages. The microbiota interacts with the human body in a symbiotic manner. Gut microbiota is involved in many bodily functions, such as the metabolism of nutrients and drugs, the regulation of many metabolic pathways, the maintenance of epithelial Integrity, the modulation of gastrointestinal motility, the stimulation and maturation of both systemic and mucosal immunity, and the production of vitamins and micronutrients. The homeostatic balance between gut microbiota and host is maintained because of several mechanisms related to structural elements of the gut, such as secretion of mucus and mucosal immunoglobulins, formation and maintenance of a mucosal barrier through secretion and replenishment of mucus, intestinal motility, mucosal and systemic immunity, interaction among different bacteria strains of bacteria and phage, and others. Many factors can break such homeostasis and/or damage the intestinal structure and functions, such as ageing, unhealthy diet, drugs (e.g., steroids or proton-pump inhibitors), and several (gastrointestinal, vascular, infectious, neurologic) diseases. Qualitative and quantitative alterations of gut microbiota may, therefore, occur, resulting in so-called dysbiosis and leading to gut microbiota-associated diseases. The alteration of gut microbiota can indeed lead to many digestive pathologies, ranging from mild to severe. Viggiano, D, et al, Eur Rev Med Pharmacol Sci 2015, 19: 1077-1085. The authors remark on the mutual dependence relationship between the intestinal barrier and the intestinal microbiota and propose that a healthy intestinal barrier plays a key role in ensuring a balanced and thriving intestinal microbiota. However, the authors admit that “[t]o date, available weapons for the maintenance and repair of gut barrier are . . . few, even if promising. Considerable efforts, including both a better understanding of the gut barrier features and mechanisms in health and disease, and the development of new pharmacological approaches for the modulation of gut barrier components, are needed for the prevention and treatment of gastrointestinal and extraintestinal diseases associated with gut barrier impairment.”

The mucus layer is part of the intestinal barrier and also serves as habitat and a source of energy for a variety of intestinal bacteria. Either directly, or through cross-talk with the gut microbiota, it is a key yet not fully considered factor for risk assessment and for management of the host's ability to cope with environmental stresses such as toxins or pollution. Gillois, K. et al, Microorganisms. 2018 Jun. 15; 6(2). pii: E53. doi: 10.3390/microorganisms6020053.

Accordingly, there is a need to maintain and restore a healthy and balanced symbiotic relationship between the gut microbiota and the human host and ensuring integrity of the intestinal barrier and a healthy and continuous mucus layer. Attempts to restore a healthy microbiota led to the development of probiotics, orally administrable preparations of bacteria, and such preparations have been shown to confer some benefit on the host. However, the administered bacteria are mostly aerobic (or facultative), limited to a few species, and thus cannot replenish the tremendous diversity in a healthy human gut. They do not appear to establish and colonize the gut. Moreover, oral administration is inefficient and only limited numbers of the Ingested bacteria reach the intestine. Thus, any benefit is limited and persists only as long as the subject consumes the probiotics.

Recently, interest has shifted to maintenance of the integrity of the intestinal barrier, not only because its integrity and selectivity are essential for the host in a direct fashion but also because it provides food and habitat for a portion of the Intestinal flora, which in turn benefits both the established microbiota and the host as the mucus colonizing bacteria displace potentially pathogenic bacteria and produce metabolic products that enhance the balance between host and microbiota. Accordingly, there is a pronounced need to devise methods and compositions for strengthening and repairing the intestinal barrier including mucous membrane, mucus layer, and gut surface area.

This can be done for example by increasing the levels/expression of barrier proteins, such as those discussed and measured in the present disclosure upon administration of compositions and practice of methods outlined below.

### SUMMARY OF THE DISCLOSURE

A human dietary supplement comprising a mixture of L-theanine, and a trace mineral complex, optionally further comprising at least one or at least two bioactive peptides, wherein the amount of L-theanine is in a daily dose within the range from about 25 mg to about 1,000 mg, the amount of trace mineral complex is in a daily dose from about 5 mg to about 1,000 mg, and the total amount of bioactive peptides, when present in the dietary supplement, is in a daily dose from about 50 mg to about 10 g wherein the dietary supplement promotes gut health.

In some embodiments, an improvement in gut health is assessed in vivo or in vitro by improvements in at least two of the following parameters: improvement in mucous membrane integrity; increase in tight junction protein expression; increase in mucin production; increase in mucin turnover; increase in microvilli-related gene transcription and/or expression; decrease in inflammatory markers and/or cytokines; increase in gut surface area; increase in short-chain fatty acid production; reduction in pH; increase in lactate production; modulation of gas production; and a favorable gut microbiota profile shift.

In some embodiments, an improvement in gut health comprises strengthening, maintenance and/or repair of the intestinal barrier and/or maintenance and/or restoration of luminal balance.

An amount of an ingredient or combination of ingredients that achieves at least one of the foregoing indicator parameters is sometimes referred to herein as an “effective amount”. In some embodiments, the amount of L-theanine is in a daily dose within the range from 50 to about 500 mg, the amount of bioactive peptides, when present in the supplement, is in a daily dose from about 100 mg to about 5 g, and the amount of trace mineral complex is in a daily dose from about 7 mg to about 300 mg. In some embodiments, the amount of L-theanine is in a daily dose within the range from about 50 mg to about 200 mg, the amount of bioactive peptides, when present in the supplement, is in a daily dose from about 200 mg to about 2 g, and the amount of trace mineral complex is in a daily dose from about 10 mg to about 100 mg. In further embodiments, the supplement further comprises one or more nondigestible carbohydrate prebiotics in an amount from about 300 mg to about 5 g if the nondigestible carbohydrate prebiotic comprises XOS, or from about 1,000 mg to about 15 g if the nondigestible carbohydrate prebiotic comprises FOS, OS or GOS. In other embodiments, the supplement further comprises a polyphenolic preparation comprising one or more and preferably two or more polyphenolic prebiotics in a daily dose within the range generally from about 10 mg to about 2 g or typically within the range from about 100 mg to about 2 g, or more specifically within the range the range of about 175 mg to about 1.5. In some embodiments, the supplement further comprises a daily dose within the range generally from about 45 mg to about 1.8 g, or typically within the range from about 60 mg to 600 g, or more specifically within the range from about 120 mg to 300 mg of a nucleotide mixture prebiotic. In yet other embodiments, the supplement further comprises, generally, from about 2 mg to about 200 mg, or typically from about 5 to about 100 mg, or more specifically from about 10 to about 50 mg of one or more phage prebiotics.

In some embodiments, the supplement further comprises an activated enzyme mixture comprising an amylase, a protease, a cellulase and a lipase and optionally further comprising one or more of a galactosidase, an Invertase, a maltase, bromelain and papain, wherein the activity of the enzymes within the following activity ranges (with the supplement typically using with the total enzyme amount being within the typical range of 25 to 600 mg:

amylase: from about 100 to about 40,000 DU/g (dextrinizing units);
protease: from about 2500 to about 120,000 HUT/g (units on a tyrosine basis)
cellulase: from about 1000 to about 100,000 CU/g (cellulase units);
lipase: from about 50 to about 1,000 FIP/g (fungal lipase international units); and, when one or more of the following is present in the supplement,
lactase: from about 20 to about 6,000 ALU/g (acid lactase units);
maltase: from about 10 to about 1000 DP/g;
bromelain: from about 100 to about 1,200 GDU/g; and
papain: from about 2,000 to about 5 million PU/g.

In certain embodiments, the amylase comprises one or more of an amylase, a fungal amylase and a glucoamylase; in other embodiments, the supplement further comprises one or more vitamins and/or added minerals and/or fermented foods. For example, the vitamins may comprise one or more of Vitamin A, Vitamin D and Vitamin 85; the added minerals may comprise one or more of zinc and selenium.

In some embodiments, the supplement further comprises: one or more prebiotics selected from the group consisting of (I) nondigestible carbohydrate prebiotics; (ii) one or more and preferably two or more polyphenolic prebiotics; and (iii) a phage prebiotic. In other embodiments the supplement further comprises an activated enzyme mixture comprising an amylase, a protease, a cellulase and a lipase a galactosidase, an invertase, a maltase, bromelain and papain, Such a supplement may have peptides which comprise at least one or preferably at least two of the following: pea peptide, mung bean peptide, Momordica charantia (bitter melon) peptide, rice peptide, algae peptide, fava bean peptide, spinach peptide, almond peptide, walnut peptide, oyster peptide, algae peptides, wheat peptide, collagen peptide, croix peptide, potato peptide, salmon peptide, corn peptide, sea cucumber peptide, yeast peptide, egg peptide, albumin peptide, milk peptide, casein peptide, amaranth peptide, silk peptide, soy peptide, oat peptide, quinoa peptide, and pulses peptides.

In some embodiments, the trace mineral complex is selected from the group consisting of a composition comprising 74 trace minerals including boron, cobalt, copper, iron, manganese, vanadium and zinc, the composition derived from rhyolitic tuff breccia also comprising volcanic glass, crystalline silicates and montmorillonite clay. In some embodiments, the phage prebiotic comprises myoviridae and siphoviridae; in other embodiments, the enzymes comprise an amylase, a protease, a cellulase, a lipase, a galactosidase, an invertase, a maltase, bromelain and papain.

The supplements of the present disclosure may be formulated in one or more daily dosages for at least 30 days. The daily dose may be achieved by a plurality of dosage forms of the same or different composition.

The mineral complex may be derived from a rhyolitic tuff breccia.

In an embodiment, methods are provided for promoting gut health comprising administering to a subject a dietary supplement as described above. Promoting gut health may comprise an improvement in at least two of the following compared to subjects not administered the supplement: an improvement in mucous membrane integrity; an increase in tight junction protein expression; an increase in TEER; an increase in mucin production; an increase in mucin turnover; an increase in microvilli-related gene transcription/expression; a decrease in Inflammatory markers and/or cytokines; an increase in gut surface area; an increase in short chain fatty acid production; a decrease in pH; an increase in lactate production; a modulation (increase or decrease) in gas production; and a gut microbiota profile shift. Stated differently, promoting gut health may comprise strengthening, maintaining and/or repairing the intestinal barrier and maintaining and/or restoring luminal balance (the chemistry of the luminal environment, including for example sufficient SCFA with Increased butyrate content, pH towards lower end of normal range, healthy mucus layer, adequate lactate, low ammonium microbial diversity and quantities). Administration of the supplement in such a method may continue for at least 30 days or indefinitely as long as benefits persist.

In particular embodiments detailed anywhere in this section the effective amount of trace mineral complex may be as detailed above (5 mg to about 1,000 mg or subranges thereof) or if needed within the range of 50 to 1,000 mg, more specifically 85 to 1000 mg. In some of these embodiments the upper limit may be 500 mg rather than 1,000 mg. The amount ranges of other ingredients, if also present, may be as provided in this section above.

In other aspects of the present disclosure, methods are provided for increasing intestinal surface area and/or Improving intestinal barrier function comprising administering to a subject an effective amount for that purpose of a composition comprising a product selected from the group consisting of trace mineral complex, L-theanine, full product (as defined below), and a combination of (a) L-theanine+bioactive peptides or (b) L-theanine+bioactive peptides+trace mineral complex or (c) L-theanine and trace mineral complex. An increase in surface area can be assessed by an increase in proteins of intestinal villi such as one or more of villin, myosin, cadherin and sucrase-isomaltase. Intestinal barrier function is assessed by assessing the expression or level of at least one of claudin-1, claudin-2, claudin-3, claudin-4, occludin and a protein of zona occludens (ZO-1), and mucin, such as mucin-3, or by assessment of TEER.

Compositions comprising an effective amount of the foregoing ingredients and combinations for increasing intestinal surface area and/or improving intestinal barrier function are also contemplated.

In yet other aspects of the present disclosure, methods are provided for increasing the efficiency of fermentation as assessed by reduced pH or increased gas production comprising administering to a subject an effective amount for that purpose of a composition comprising full product (as defined below), preferably on a daily basis. Compositions comprising the full product in an amount effective for this purpose are also contemplated.

In still other aspects of the present disclosure, methods are provided for increasing the efficiency of fermentation as assessed by increased of one or more SCFA (acetate propionate and butyrate) or total SCFA and/or lactate levels comprising administering to a subject an effective amount for that purpose of a composition comprising a product selected from the group consisting of L-theanine, full product (as defined below), a combination of (a) L-theanine+bioactive peptides or (b) L-theanine+bioactive peptides+trace mineral complex or (c) L-theanine and trace mineral complex, preferably on a daily basis. Compositions comprising one or more of the foregoing products in an amount effective for this purpose are also contemplated.

In other aspects, methods are provided for increasing the abundance of members of the intestinal bacterial community (including one or more of Bacteroidetes or Firmicutes phyla or one or more of lactobacilli or bifidobacteria, or Feacalibacterium prausnitzii) comprising administering to a subject a composition comprising the full product), or a combination of (a) L-theanine+bioactive peptides or (b) L-theanine+bioactive peptides+trace mineral complex, preferably on a daily basis. Compositions comprising one or more of the foregoing products in an amount effective for this purpose are also contemplated.

Provided is a dietary supplement composition comprising one or both of L-theanine and a trace mineral complex, optionally in combination with bioactive peptides, in an amount or amounts individually or in combination effective to promote gut health. In some embodiments, the amount or amounts is/are effective individually or in combination to effect one or more of: an increase in membrane integrity; an increase in tight junction protein expression (and/or a decrease in claudin-2); an increase in mucin production; an increase in mucin turnover; an increase in microvilli-related gene transcription/expression; a decrease in inflammatory markers and/or cytokines; an increase in gut surface area; an increase in short chain fatty acid production; a decrease in pH; an increase in lactate and gas production; and a gut microbiota profile shift.

In further embodiments, the amount or amounts is/are effective to accomplish one or more of the following: Increase microbial fermentation, enrich microbial community, increase microbial diversity, fortify intestinal barrier function, and increase intestinal surface area. More specifically, benefits contemplated include one or more of
increase of the amount of total short-chain fatty acid production or of at least one of propionate, acetate and butyrate;
increase of the expression or level of at least one tight junction protein (for example, claudin-1, claudin-3, claudin-4, occludin, or a zona occludens protein) or mucin-3 or decrease the expression or level of claudin-2;
increase in transepithelial electrical resistance;
increase in the abundance or diversity of a member or phylum of the intestinal bacterial community of the intestinal lumen or mucus area (for example, Firmicutes, Bacteroidetes, Lactobacilli, Bifidobacteria, F. prausnitzii, or A. muciniphila);
increase in the expression or level of at least one intestinal surface area marker protein (for example, villin, myosin, cadherin or sucrase isomaltase).

In some embodiments, the amount of trace mineral complex is in a daily dose within the range of 50 to 1,000 mg and preferably 85 to 1000 mg; the amount of L-theanine is in a daily dose within the range from about 25 mg to about 1,000 mg, and the amount of bioactive peptides, when present in the dietary supplement, is in a daily dose from about 50 mg to about 10 g.

Optional additional components of the present supplements include one or more of:
one or more and preferably two or more polyphenolic prebiotics in a daily dose within the range generally from about 10 mg to about 2 g or typically within the range from about 100 mg to about 2 g, or more specifically within the range the range of about 50 mg to about 1.5 g;
one or more nondigestible carbohydrate prebiotics in an amount from about 300 mg to about 5 g if the nondigestible carbohydrate prebiotic comprises XOS (which is preferred), or from about 1,000 mg to about 15 g if the nondigestible carbohydrate prebiotic comprises FOS, OS or GOS; and
one or more nucleotide mixture prebiotic in a daily dose within the range generally from about 45 mg to about 1.8 g, or typically within the range from about 60 mg to 1 g, or more specifically within the range from about 120 mg to 600 mg;
one or more phage prebiotics in an amount within the range from about 2 mg to about 200 mg, or typically from about 5 to about 100 mg, or more specifically from about 10 to about 50 mg.

Additional optional components may include inter alia:
an activated enzyme mixture comprising an amylase, a protease, a cellulase and a lipase and optionally further comprising one or more of a galactosidase, an invertase, a maltase, bromelain and papain, wherein the activity of the enzymes is within the following ranges:
   amylase: from about 100 to about 40,000 DU/g (dextrinizing units);
   protease: from about 2500 to about 120,000 HUT/g (units on a tyrosine basis)
   cellulase: from about 1000 to about 100,000 CU/g (cellulase units);
   lipase: from about 50 to about 1,000 FIP/g (fungal lipase international units); and, when one or more of the following is present in the supplement:
   lactase: from about 20 to about 6,000 ALU/g (add lactase units);
   maltase: from about 10 to about 1000 DP/g;
   bromelain: from about 100 to about 1,200 GDU/g; and
   papain: from about 2,000 to about 5 million PU/g.

Other optional ingredients include vitamins and/or added minerals and/or fermented foods.

In particular aspects a method is provided for increasing intestinal surface area and/or improving intestinal barrier function comprising administering to a subject an effective amount for that purpose of a composition comprising a product selected from the group consisting of a trace mineral complex, L-theanine, a combination of (a) L-theanine+bioactive peptides or (b) L-theanine+bioactive peptides+trace mineral complex or (c) L-theanine and trace mineral complex; and a combination of L-theanine+bioactive peptides+trace mineral complex+optionally, one or more of (i) a polyphenol prebiotic, (ii) a nondigestible fiber prebiotic, and (iii) a polynucleotide.

In some embodiments of the Immediately preceding method, the increase in surface area is assessed by an increase in one or more proteins of intestinal villi such as one or more of villin, myosin, cadherin and sucrase-isomaltase; and/or intestinal barrier function is assessed by assessing the expression or level of at least one of claudin-1, claudin-2, claudin-3, claudin-4, occludin and a protein of zona occludens (such as ZO-1), and mucin, such as mucin-3, or by a decrease in claudin-2, or by an increase in TEER.

In another aspect, a method is provided for increasing the efficiency of fermentation comprising administering to a subject an effective amount for that purpose of a composition comprising one or more of the following: a trace mineral complex, L-theanine, a combination of (a) L-theanine+bioactive peptides or (b) L-theanine+bioactive peptides+trace mineral complex or (c) L-theanine and trace mineral complex; and a combination of L-theanine+bioactive peptides+trace mineral complex+optionally, one or more of (i) a polyphenolic prebiotic preparation. (ii) a nondigestible fiber prebiotic, and (ii) a polynucleotide each preferably on a daily basis.

The method the efficiency of fermentation may be assessed inter alia by increased levels of one or more SCFA (acetate, propionate and butyrate) or total SCFA and/or lactate levels or pH decrease or gas production.

In an additional aspect, a method is provided for increasing the abundance or diversity of members of the intestinal bacterial community (including one or more of Bacteroidetes or Firmicutes phyla or one or more of Lactobacilli or Bifidobacteria, or Faecalibacterium prausnitzii) comprising administering to a subject a composition comprising one or more of the following: a trace mineral complex, L-theanine, a combination of (a) L-theanine+bioactive peptides or (b) L-theanine+bioactive peptides+trace mineral complex or (c) L-theanine and trace mineral complex; and a combination of L-theanine+bioactive peptides+trace mineral complex+optionally, one or more of (i) a polyphenol prebiotic, (ii) a nondigestible fiber prebiotic, and (iii) a polynucleotide, each preferably on a daily basis.

In yet an additional aspect, a method is provided for increasing abundance and/or diversity of or increasing the efficiency of fermentation by an intestinal microbial community the method comprising administering to a subject an amount effective for that purpose of a composition comprising a polyphenolic preparation containing one I or more and preferably two or more polyphenolic prebiotics, the amount is effective to accomplish at least one of the following: Increasing lactate production and Increasing abundance of at least one of bifidobacteria and F. prausnitzii. In some embodiments, the amount of polyphenolic preparation is in a daily dose within the range generally from about 10 mg to about 2 g or typically within the range from about 100 mg to about 2 g, or more specifically within the range the range of about 50 mg to about 1.5 g.

The disclosure is further illustrated by one or more of the drawings (figures) identified below, which are illustrative and not limiting.

### BRIEF DESCRIPTION OF THE FIGURES

FIG. 1. Gas production during different time-intervals (0-6 h, 6-24 h and 24-48 h) upon fermentation of the full product and selected subfractions by the gut microbiota of a single healthy adult donor. A negative control (blank) and a positive control (inulin) were also included.

FIG. 2. Total SCFA production during different time-intervals (0-6 h, 6-24 h and 24-48 h) upon fermentation of the full product and selected subfractions by the gut microbiota of a single healthy adult donor. A negative control (blank) and a positive control (inulin) were also included.

FIG. 3. Acetate production during different time-intervals (0-6 h, 6-24 h and 24-48 h) upon fermentation of the full product and selected subfractions by the gut microbiota of a single healthy adult donor. A negative control (blank) and a positive control (inulin) were also included.

FIG. 4. Propionate production during different time-intervals (0-6 h, 6-24 h and 24-48 h) upon fermentation of the full product and selected subfractions by the gut microbiota of a single healthy adult donor. A negative control (blank) and a positive control (inulin) were also included.

FIG. 5. Butyrate production during different time-intervals (0-6 h, 6-24 h and 24-48 h) upon fermentation of the full product and selected subfractions by the gut microbiota of a single healthy adult donor. A negative control (blank) and a positive control (inulin) were also Included.

FIG. 6. Branched SCFA production during different time-intervals upon fermentation of the full product and selected subfractions by the gut microbiota of a single healthy adult donor. A negative control (blank) and a positive control (inulin) were also included.

FIG. 7. Ammonium production during different time-intervals upon fermentation of full product and selected subfractions by the gut microbiota of a single healthy adult donor. A negative control (blank) and a positive control (inulin) were also included.

FIG. 8. Lactate production and consumption during different time-intervals (0-6 h, 6-24 h and 24-48 h) upon fermentation of the full product and selected subfractions by the gut microbiota of a single healthy adult donor. A negative control (blank) and a positive control (Inulin) were also included.

FIG. 9. Abundance of Bifidobacterium sp. in the lumen on different timepoints of the incubation upon fermentation of the full product and selected subfractions by the gut microbiota of a single healthy adult donor. A negative control (blank) and a positive control (inulin) were also included.

FIG. 10. Abundance of Bifidobacterium sp. in mucus on different timepoints of the Incubation upon fermentation of the full product and selected subfractions by the gut microbiota of a single healthy adult donor. A negative control (blank) and a positive control (inulin) were also included.

FIG. 11. Abundance of Lactobacillus sp. In the lumen on different timepoints of the Incubation upon fermentation of the full product and selected subfractions by the gut microbiota of a single healthy adult donor. A negative control (blank) and a positive control (inulin) were also included.

FIG. 12. Abundance of Lactobacillus sp. In mucus on different timepoints of the incubation upon fermentation of the full product and selected subfractions by the gut microbiota of a single healthy adult donor. A negative control (blank) and a positive control (inulin) were also included.

FIG. 13. Abundance of Bacteroidetes in the lumen on different timepoints of the incubation upon fermentation of the full product and selected subfractions by the gut microbiota of a single healthy adult donor. A negative control (blank) and a positive control (Inulin) were also included.

FIG. 14. Abundance of Bacteroidetes in mucus on different timepoints of the incubation upon fermentation of the full product and selected subfractions by the gut microbiota of a single healthy adult donor. A negative control (blank) and a positive control (inulin) were also included.

FIG. 15. Abundance of Faecalibacterium prausnitzii in the lumen on different timepoints of the incubation upon fermentation of the full product and selected subfractions by the gut microbiota of a single healthy adult donor. A negative control (blank) and a positive control (inulin) were also included.

FIG. 16. Abundance of Faecalibacterium prausnitzii in mucus on different timepoints of the incubation upon fermentation of the full product and selected subfractions by the gut microbiota of a single healthy adult donor. A negative control (blank) and a positive control (inulin) were also included.

FIG. 17. Abundance of Firmicutes in the lumen (top) on different timepoints of the Incubation upon fermentation of the full product and selected subfractions by the gut microbiota of a single healthy adult donor. A negative control (blank) and a positive control (inulin) were also included.

FIG. 18. Abundance of Firmicutes in mucus on different timepoints of the incubation upon fermentation of the full product and selected subfractions by the gut microbiota of a single healthy adult donor. A negative control (blank) and a positive control (inulin) were also included.

FIG. 19. Effect of batch samples on transepithelial electrical resistance (TEER) of TNFα/IFNγ-treated Caco-2/HT29-MTX co-cultures. TEER was measured 24 h (A) and 48 h (B) after apical treatment of the co-cultures with batch samples or the positive control Sodium butyrate (NaB) in combination with basolateral stimulation with TNFα/IFNγ. Each value was normalized to its corresponding 0 h value and is shown as percentage of initial value. The dotted line represents 100% (initial value). Data are plotted as mean±SEM (CM, TNFα/IFNγ, NaB and blank) or as single measurements (treatment batch samples). CM=complete medium control.

FIG. 20. Effect of colonic batch samples on the expression of the barrier-strengthening tight junction genes in a Caco-2/HT29-MTX co-culture model. Cells were apically treated with the colonic batch samples or the positive control Sodium butyrate in combination with basolateral stimulation with TNFα/IFNγ for 48 h. Gene expression was determined via qPCR for claudin-1 (A), claudin-3 (B), claudin-4 (C) and occludin (D). The grey dotted line represents the TNFα/IFNγ control. The black dotted line represents the blank control. Data are plotted as mean±SEM (CM, TNFα/IFNγ, NaB and blank) or as single measurements (treatment batch samples). CM=complete medium control.

FIG. 21. Effect of colonic batch samples on the expression of the permeability-inducing tight junction gene claudin-2 in a Caco-2/HT29-MTX co-culture model. Cells were apically treated with the colonic batch samples or the positive control Sodium butyrate in combination with basolateral stimulation with TNFα/IFNγ for 48 h. Gene expression was determined via qPCR. The grey dotted line represents the TNFα/IFNγ control. The black dotted line represents the blank control. Data are plotted as mean±SEM (CM, TNFα/IFNγ, NaB and blank) or as single measurements (treatment batch samples). CM=complete medium control.

FIG. 22. Effect of colonic batch samples on the expression of the mucin-3 gene in a Caco-2/HT29-MTX co-culture model. Cells were apically treated with the colonic batch samples or the positive control Sodium butyrate in combination with basolateral stimulation with TNFα/IFNγ for 48 h. Gene expression was determined via qPCR. The grey dotted line represents the TNFα/IFNγ control. The black dotted line represents the blank control. Data are plotted as mean±SEM (CM, TNFα/IFNγ, NaB and blank) or as single measurements (treatment batch samples). CM=complete medium control.

FIG. 23. Results of the Villin expression in CaCo-2 cells after treatment with the test blend or cell culture medium only for 6 days (A) or 12 days (B). Means of two independent experiments are shown. Results in ng Villin/mg protein±SEM. The line drawn marks the level of the untreated control (Medium).

FIG. 24. Results of the Myosin 1 expression in CaCo-2 cells after treatment with the test blend or cell culture medium only for 6 days (A) or 12 days (B). Means of two independent experiments are shown. Results in ng Myosin/mg protein±SEM. The line drawn marks the level of the untreated control (Medium).

FIG. 25. Results of the Cadherin expression in CaCo-2 cells after treatment with the test blend or cell culture medium only for 6 days (A) or 12 days (B). Means of two independent experiments are shown. Results in ng Cadherin/mg protein t SEM. The line drawn marks the level of the untreated control (Medium).

FIG. 26. Results of the Sucrase-isomaltase expression in CaCo-2 cells after treatment with the test blend or cell culture medium only for 6 days (A) or 12 days (B). Means of two independent experiments are shown. Results in ng SI/mg protein±SEM. The line drawn marks the level of the untreated control (Medium).

FIG. 27. Results of the Claudin 1 expression in CaCo-2 cells after treatment with the test blend or cell culture medium only for 6 days (A) or 12 days (B). Means of two independent experiments are shown. Results in pg Claudin/mg protein t SEM. The line drawn marks the level of the untreated control (Medium).

FIG. 28. Results of the Occludin 1 expression in CaCo-2 cells after treatment with the test blend or cell culture medium only for 6 days (A) or 12 days (B). Means of two independent experiments are shown. Results in pg Occludin/mg protein±SEM. The line drawn marks the level of the untreated control (Medium).

FIG. 29. Results of the Zona occludens 1 (ZO-1) protein expression in CaCo-2 cells after treatment with the test blend or cell culture medium only for 6 days (A) or 12 days (6). Means of two independent experiments are shown. Results in pg ZO-1/mg protein t SEM. The line drawn marks the level of the untreated control (Medium).

### DETAILED DESCRIPTION

“Subject” means a human subject to be treated. The definition includes subjects considered healthy in that they present no clinical symptoms of disease but suffer from transient or chronic gastrointestinal (GI) distress, which indicates suboptimal or compromised gut health. Symptoms of GI distress vary in severity and duration from irritable bowel disorders (IBD) at the most serious chronic extreme, to diarrhea, constipation (symptoms that can be either transitory or chronic), to more transitory and common issues like gas, bloating, rumbling, feeling of fullness and abdominal discomfort. The definition of “subject” also includes subjects with a compromised Intestinal microbiome and/or intestinal barrier, whether as a result of a poor diet, age, treatment with drugs or associated with a disease. Lastly, the definition of “subject” includes those who do not present with symptoms but are likely to have suboptimal gastroenterological function, whether because of aging, disease, medication or simply poor diet and are thus at risk of developing gastrointestinal disorders and the aforementioned chronic or transient types.

Administration of the present dietary supplements is to promote gut health by favorably affecting at least one or preferably at least two of the following parameters of intestinal structure: increase mucous membrane integrity; increase tight junction protein level or expression; increase TEER; increase mucin production; increase mucin turnover; increase microvilli-related gene transcription/expression; increase intestinal surface area marker proteins; decrease inflammatory markers and inflammatory cytokines (such as one or more of the following TNF-alpha, IFN-gamma, IL-1b); decrease pH while maintaining it within physiologic limits for e.g., the colon, increase short chain fatty acid (SCFA) production and/or tilt the balance of SCFA in favor of butyrate; modulate (increase or decrease as needed) lactate production; modulate (increase or decrease as needed) gas production; and shift microbial profile away from Firmicutes towards Bacteroidetes (decreasing F/B ratio but maintain or rebalance specific beneficial bacterial populations within the Firmicutes phylum, especially those that colonize or more generally are closely associated with the mucosal layer and/or consume mucus). The goal is to strengthen or repair the barrier function of the intestinal epithelium (mucus membrane) including the mucus layers and tight junction, and thereby to treat (including ameliorate) or prevent (including avoiding or postponing the manifestation of symptoms) both transient and chronic intestinal disorders (exemplified above) and extraintestinal disorders associated with gut barrier impairment. Indeed, conditions apparently unrelated to GI distress or dysfunction, such as depression, allergic disorders, psoriasis and eczema have been found to correlate with suboptimal or impaired GI function. In addition, an improvement in gut structure may improve the ability of the treated subject to cope with physiological, emotional and environmental stress, the latter including for example toxins and pollution. In other words, the present dietary supplements are not aimed only at subjects presenting with symptoms but at all subjects who may have suboptimal gastroenterological function and are thus at risk of developing gastrointestinal disorders and the aforementioned chronic or transient symptoms such as pain, bloating, discomfort, stool consistency, straining and constipation, diarrhea etc.

These parameters can be assessed in vivo, for example by obtaining fecal or tissue samples or using an appropriate animal model, or in vitro, using cell culture, in vitro simulations of the colon, or assays using in vitro propagated intestinal tissue. Exemplary methods of such assessment are provided below.

The dosage ranges and formulations for the dietary supplements of the present disclosure are provided below. The formulations, whether tablets, powder, capsules, suspensions or any other form suitable for oral administration are prepared by known techniques. The supplements may be taken daily, once per day or in two or three divided doses, with or without food. Thus, when a daily dose is specified, it can be achieved by administering multiple dosage forms. Each dosage form may contain all the specified components or some dosage forms may contain one or more components and others the remainder, or may contain the same component in varying amounts. For example, a human dietary supplement comprising a mixture of L-theanine and a trace mineral complex, and further comprising at least one or at least two bioactive peptides, wherein the amount of L-theanine in a daily dose is within the range from about 25 mg to about 1,000 mg, the amount of trace mineral complex is in a daily dose from about 5 mg to about 1,000 mg, and the amount of bioactive peptides, is in a daily dose from about 50 mg to about 10 g is not limited to a single unit dosage form but may be in different dosage forms that together will constitute a daily dose. Treatment should be instituted for at least 30 days (even though benefits may become manifest to the subject or the physician earlier, for example by amelioration or elimination of some or all symptoms) and may continue for at least 6 months or even indefinitely as long as benefits are maintained.

Whereas some of the components of the present supplements have been reported to affect at least one parameter mentioned above, to the knowledge of the present inventors, effects on gut structure, notably structure of the small and large intestine, such as mucous membrane barrier, including tight junction, permeability (as measured for example by mucus membrane electrical resistance) mucus layer (in turn related to mucus production and/or turnover), and gut surface area (villi, microvilli) are not believed to have been reported for either theanine or trace mineral complexes, individually or as a combination. Nor have they been reported for 3-component combinations of the present disclosure, i.e., as a further combination of theanine and trace mineral complex with bioactive peptides, especially but not exclusively peptides of plant origin. The present supplements and methods of their use are responsive to the pronounced need in the art to reinforce or repair the barrier function of the intestinal surface and/or to maintain or restore balance in the gut microbiome.

L-Theanine

L-theanine (chemically known as γ-glutamylethylamide) is a non-proteinogenic amino acid of tea that takes part in the biosynthesis of its polyphenols. Recently discovered neuroprotective effects of L-theanine can be attributed to its structural similarity with glutamate, the principal excitatory neurotransmitter in brain. This amino acid also displays antioxidant and anti-inflammatory properties, improves motor behavioral abnormalities, increases dopamine availability and may cause a favorable downshift in neurodegeneration due to glutamate excitotoxicity. It has not been extensively studied for any effect on gut structure or ecology of gut microbiota.

The daily amount of L-theanine in the present supplement is generally within the range of about 25 to about 1,000 mg, typically from about 50 to about 500 mg, and more specifically from about 50 to about 200 mg.

Trace Mineral Complex or Blend

The role of certain trace minerals in gut health has been attributed to a contribution by trace minerals such as zinc, molybdenum, copper and manganese to the enzymatic action in the digestive process.

The trace mineral complex or blend can be provided in the form of a commercially available mixture of microminerals, such as those available under the tradename Concentrace® (a proprietary concentrated preparation of 72 minerals isolated from a naturally occurring trace mineral mixture from lakebed, available from Trace Minerals Research, Roy, Utah 8; or Aquamin® (containing a concentrated mixture of 77 minerals isolated from a calcareous marine algae (Lithothamnion sp.) harvested from seabed) from Marigot Ltd., Strand Farm, Currabinny, Carrigaline, Co. Cork, Ireland; or Youngevity® plant-derived minerals, a proprietary plant mineral blend available from Youngevity Chula Vista, Calif. or RhyoTrace™ (a proprietary 74-mineral complex of volcanic and ancient seabed origin derived from rhyolitic tuff breccia, characterized as a hydrated potassium-sodium calcium aluminosilicate, providing trace minerals such as boron, cobalt, copper, iron, manganese, vanadium and zinc and many others, and containing, in addition to the minerals, volcanic glass, crystalline silicate minerals and montmorillonite clay) available from Life Minerals LLC., Nephi, Utah; or Plant Sourced Minerals (70+minerals sourced from Senonian Vegetate) available from Natural Vitality, Austin Tex.; or Colloidal Life™ trace minerals, a combination of 72 colloidal minerals and electrolytes of aquatic and plant origin available from Source Naturals Scotts Valley, Calif. 95066.

The daily amount of trace mineral blend provided in the present dietary supplements is within the general range from about 5 mg to about 1,000 mg daily, typically from about 7 mg to about 300 mg and more specifically from about 10 mg to about 100 mg. Without wishing to be bound by theory, it is believed that the mineral complex can be provided in suboptimal amounts, i.e., at doses substantially below the total daily doses recommended for mineral supplementation, which may indicate a potentiation mechanism, but higher doses may also be administered as provided above.

Trace mineral complex or blend supplements have not been reported previously to have been associated with strengthening or repair of the Intestinal barrier or more specifically the mucus layer and/or tight junction or with Improving the balance of luminal microbiota or creating a balanced luminal environment (increased presence of SCFA with Increased proportion of butyrate, lower pH, modulation of lactate, healthy mucus layer).

Bioactive Peptides

Bioactive peptides, particularly those derived from food of plant or animal (including fish) origin, to be included in the dietary supplement of the present disclosure include one or more and preferably two or more of the following: pea peptide, mung bean peptide, Momordica charantia (bitter melon) peptide, rice peptide, fava bean peptide, spinach peptide, broccoli peptide, almond peptide, wakame peptide, walnut peptide, oyster peptide, algae peptides (one or more of several genera and species including without limitation spirulina), wheat peptide, collagen peptide, croix peptide, potato peptide, salmon peptide, corn peptide, maize peptide, sea cucumber peptide, yeast peptide, egg peptide, albumin peptide, milk peptide, casein peptide, sunflower peptide, sesame peptide, amaranth peptide, silk peptide, soy peptide, oat peptide, quinoa peptide, as well as pulses peptides, such as hemp, lentil, bean, fava bean and chick pea peptides. In general, plant peptides and peptides derived from marine organisms, such as algae, fish or shellfish are preferred, with plant peptides being most preferred.

Several of the foregoing peptides are commercially available. Others can be prepared from plant and animal proteins by enzymatic hydrolysis of proteins or by fermentation, and purified, as is known in the field. See, e.g., Chakrabarti, S., et al, Nutrients. 2018 November; 10(11): 1738 (published online 2018 Nov. 12. doi: 10.3390/nu10111738) and references cited therein including: Korhonen, H., et al, Bioactive peptides: Production and functionality. Int. Dairy J. 2006; 16:945-960. doi: 10.1016/j.idairyj.2005.10.012; Daliri E. B. M., et al, Bioactive Peptides. Foods. 2017; 6:32. doi: 10.3390/foods6050032; Gobbetti M., et al, Production of Angiotensin-I-Converting-Enzyme-Inhibitory Peptides in Fermented Milks Started by Lactobacillus delbrueckii subsp. bulgaricus 551 and Lactococcus lactis subsp. cremoris FT4. Appl. Environ. Microbiol. 2000; 66:3898-3904. doi: 10.1128/AEM.66.9.3898-3904.2000; EI-Fattah A. M. A., et al, Bioactive peptides with ACE-1 and antioxidant activity produced from milk proteolysis. Int. J. Food Prop. 2017; 20:3033-3042. doi: 10.1080/10942912.2016.1270963; Ferri M., et al, Peptide fractions obtained from rice by-products by means of an environment-friendly process show in vitro health-related bioactivities. PLoS ONE. 2017; 12:e0170954. doi: 10.1371/journal.pone.0170954; Fu Y., et al, Structural characteristics of low bitter and high umami protein hydrolysates prepared from bovine muscle and porcine plasma. Food Chem. 2018; 257:163-171. doi: 10.1016/j.foodchem.2018.02.159; Mora L, et al, Effect of cooking and simulated gastrointestinal digestion on the activity of generated bioactive peptides in aged beef meat. Food Funct. 2017; 8:4347-4355. doi: 10.1039/C7FO01148B; Aspri M., Bioactive properties of fermented donkey milk, before and after in vitro simulated gastrointestinal digestion. Food Chem. 2018; 268:476-484. doi: 10.1016/j.foodchem.2018.06.119; Vieira E. F., et al, Impact of in vitro Gastrointestinal Digestion and Transepithelial Transport on Antioxidant and ACE-inhibitory Activities of Brewer's Spent Yeast Autolysate. J. Agric. Food Chem. 2016; 64:7335-7341. doi: 10.1021/acs.jafc.6b02719; Picarlello G., et al, Role of intestinal brush border peptidases in the simulated digestion of milk proteins. Mol. Nutr. Food Res. 2015; 59:948-956. doi: 10.1002/mnfr.201400856; Rizzello C. G., et al, Improving the antioxidant properties of quinoa flour through fermentation with selected autochthonous lactic acid bacteria. Int. J. Food Microbiol. 2017; 241:252-261. doi: 10.1016/j.ijfoodmicro.2016.10.035; Aguilar-Toalá J. E., et al, Assessment of multifunctional activity of bioactive peptides derived from fermented milk by specific Lactobacillus plantarum strains. J. Dairy Sci. 2017; 100:65-75. doi: 10.3168/jds.2016-11846; Ahn J. E., et al, Angiotensin I-Converting Enzyme (Ace) Inhibitory Peptides From Whey Fermented By Lactobacillus Species. J. Food Biochem. 2009; 33:587-602. doi: 10.1111/j.1745-4514.2009.00239.x.; Sanjukta S., et al, Enhancement of antioxidant properties of two soybean varieties of Sikkim Himalayan region by proteolytic Bacillus subtilis fermentation. J. Funct. Foods. 2015; 14:650-658. doi: 10.1016/j.jff.2015.02.033; Chaudhury A., et al, Clinical review of antidiabetic drugs: Implications for type 2 diabetes mellitus management. Front. Endocrinol. 2017; 8:6. doi: 10.3389/fendo.2017.00006.

The daily amount of peptides, preferably plant peptides, provided in the present dietary supplements is within the general range of about 50 mg to about 10 g, typically about 100 mg to about 5 g, and more specifically about 200 mg to about 2 g.

Prebiotics

Nondigestible Carbohydrate Prebiotics

One or more dietary fiber prebiotics, carbohydrate polymers which are nondigestible by humans, may optionally and advantageously be included in the present dietary supplements, in addition to the trace-mineral-complex/theanine or trace mineral/theanine/peptide (preferably plant peptide) combination. These may include one or more of non-digestible oligosaccharides including, XOS (xylooligosaccharides), FOS (fructooligosaccharides), GOS (galactooligosaccharides), and/or AXOS (arabinoxylo-oligosaccharides), IMO (isomaltooligosaccharides). 105 (inhibitory oligosaccharides, inhibiting cellulase enzymes).

Prebiotics in general, used Individually, can selectively stimulate the growth of Bifidobacteria and Lactobacilli, leading to production of short chain fatty acids (SCFAs), and consequent lowering of gut pH (which reduces levels of pathogens and enhances absorption of calcium (Ca2+) and magnesium (Mg2+). The SCFAs lower pH and may suppress formation of toxic bacterial metabolites.

Prebiotics, being nondigestible, are resistant to gastric acidity and hydrolysis by human enzymes; but they are fermented by microbiota, and selectively stimulate the growth and/or activity of intestinal bacteria associated with health and well-being.

The main bacterial fermentation products of complex carbohydrates are SCFA, mainly acetate, propionate and butyrate. SCFA are important indicators of bacterial fermentation in the colon. The concentration of SCFAs changes throughout the length of the GI tract, with the highest concentrations in the proximal colon and diminishing concentrations in the distal colon, the region of the gastrointestinal tract with the greatest density of microbes.

Butyrate is the key energy source for colonocytes and enterocytes. In a healthy GI tract SCFAs are absorbed in the gut or used by the microbiota. SCFAs also influence gastrointestinal epithelial cell integrity, barrier function, inflammation, glucose homeostasis, lipid metabolism, appetite regulation and immune function. Additionally, particular SCFAs, including butyrate have demonstrated a critical role in optimal ileal and colonic motor activity, which is a modulation of gut structure and function. In general, a higher amount or butyrate, or a higher proportion among SCFA, is favorable.

Other metabolic activities include the activation or inactivation of bioactive food components like isoflavonoids, flavonoids and plant lignans, the conversion of pro-drugs to drugs, the production of vitamins and the transformation of bile acids and xenobiotics.

The extent of prebiotic fermentation has been shown to depend not only upon the type of fiber or the microbiota composition, but also to be significantly altered by interaction with the host (genetics), lumen environment (pH and current microbiota composition) and the amount of prebiotic. Thus, the capacity for fermentation of carbohydrate contained in a specific prebiotic per individual may vary from 20% to 100%. Lumen environment and gut structure, which mediates host interaction, may have a significant impact on fermentation properties, including effective dose, of a particular microbial profile.

SCFAs also account for 2-10% of the total energy consumption in humans, are the main energy source for large intestinal epithelial cells, and affect the production of mucins (mucus). In addition, SCFAs physiologically influence blood flow to the colon mucous membrane, the absorption of fluids and electrolytes, the autonomic nervous system, and the secretion of gut hormones. A considerable part of the beneficial effect of prebiotics is thought to be due to SCFAs produced by intestinal microbes. Research has shown that the concentration of SCFAs is 70.140 mmol/L in the proximal colon and 20.70 mmol/L in the distal colon in healthy adults. In general, acetate is thought to be more prevalent, followed by propionate and butyrate. Animal experiments have shown that the total amount of SCFAs may be approximately 400400 mmol/day when 60 g/day of undigested carbohydrates reach the colon. (Ohira, H. et al, J Atheroscier Thromb. 2017 Jul. 1; 24(7): 660-672; doi: 10.5551/jat.RV17006). SCFAs contribute the maintenance of a balanced luminal environment (luminal balance).

Although it is the least abundant SCFA produced (˜60% acetate, 25% propionate, and 15% butyrate in humans) (6, 7), butyrate is the major energy source for colonocytes.

Thus, carbohydrate prebiotics have the potential to confer substantial benefits to the subject taking such supplements but this potential is realized only if the intestinal microbiota of the host is able to elaborate efficient fermentation of the prebiotics, and the host is able to use such products efficiently. The present inventors contemplate that the efficiency of fermentation will increase if the prebiotic is co-administered (i.e., administered substantially contemporaneously, though not necessarily as part of the same composition) with the foregoing combination of theanine and trace mineral complex, or theanine, trace mineral complex and (preferably plant) peptide.

The present disclosure thus contemplates inclusion of one or more dietary nondigestible carbohydrate oligosaccharide prebiotics in further combination with the trace mineral complex/theanine/peptide combination in a daily amount within the range:

XOS: generally, from about 300 mg to about 5 g; typically, from about 500 mg to about 3 g; and more specifically, from about 800 mg to 1200 mg.
GOS, FOS, including inulin, MOS, IOS, each, general range: about 1000 mg to about 15 g; typical range from about 600 mg to about 10 g; more specific range from about 2 g to about 5 g.

Other Prebiotics

Included in this group are components of the present dietary supplement that contribute to growth of the intestinal microbiota other than the foregoing prebiotic components containing complex oligosaccharides (nondigestible carbohydrates). Such additional prebiotic components include polyphenols, nucleotides (also referred to as polynucleotides) and phage. One or both of these can be added to the trace mineral complex/theanine or mineral complex/theanine/peptide combination instead of or in addition to the foregoing nondigestible carbohydrate prebiotics.

Polyphenol Mixture

Included in this component are soluble polyphenolic preparations derived from plants and fruit and enriched in polyphenolic compounds such as flavonoids, including flavones, flavanones, flavonones, flavanols, flavonols, flavononols, isoflavones, and anthocyanins. And nonflavonoids phenolics, including phenolic acids, hydrolysable tannins and stilbenes. The polyphenolic preparations are typically in the form of powders or extracts from plants and fruit, such as two, three, four or more (or all) of apple extract, purple sweet potato extract, mangiferin, berberine, cocoa polyphenols/flavanois extract, quercetin, cranberry fruit powder or extract, avocado fruit powder or extract, skullcap root extract, a source of baicalin, passion fruit extract, a source of chrysin, blueberry fruit extract, pomegranate fruit extract (a source of punicalagins), sea buckhorn fruit, and turmeric extract, a source of curcuminoids.

The total amount of the polyphenol preparation will generally be in the range from about 10 mg to about 2 g, typically in the range from about 100 mg to about 2 g, and more specifically in the range of about 175 mg to about 1.5 g based on the plant, or fruit-derived material. The milligram amounts of each constituent of this polyphenolic component can be the same or different as Indicated in the art.

Nucleotides

Dietary nucleotide supplementation has been associated with relief of constipation and prebiotic effect (Pinheiro et al. BMC Complementary and Alternative Medicine (2017) 17:441; as well as improvement in body growth, nonspecific immune activity and intestine length (Li, X et al, Animal Nutrition, 1 (2015) 244-251 (experiments with tilapia fish)). Elsewhere, it is reported that nucleotide supplementation showed the following activities in the intestine: proliferation and differentiation of intestinal cell lines; increased villus height; increase of IgA production and other beneficial immune system effects (proliferation and differentiation of T-cells and macrophages, activation of immune signaling pathways). See, e.g., Che, L. et al, PLOS ONE | DOI:10.1371/journal.pone.0157314 Jun. 15, 2016 reporting on experiments conducted in neonatal piglets. Gil, A. European Journal of Clinical Nutrition (2002) 56, Suppl 3, S1-S4 also report that nucleotides influence lymphocyte maturation, activation and proliferation; affect the lymphocyte subset populations in both the small intestine and blood; are involved in enhancing macrophage phagocytosis and delayed hypersensitivity as well as allograft and tumor responses; and contribute to the immunoglobulin response in early life, having a positive effect on infection. The author also states however that the molecular mechanisms by which dietary nucleotides modulate the immune system are practically unknown.

Nutritional yeast, Saccharomyces cerevisiae, is an example of a yeast that is appropriate for use in the present dietary supplements as a source of polynucleotides. Brewer's yeast, another strain of S. cerevisiae, is another example. Nucleotide preparations from yeast for use as supplements are commercially available.

The daily amount of the nucleotide component of the present dietary supplements is generally within the range from about 45 mg to about 1.8 g, typically from about 60 mg to 600 mg, and more specifically from about 120 mg to 300 mg based on commercially available nucleotide supplements containing about 60 to about 70% polynucleotides.

Bacteriophates

Bacteriophages, such as myoviridae, podoviridae and siphoviridae, though not technically a prebiotic, have been shown to act as prebiotics in that they precipitate increases in populations of beneficial gut bacteria such as one or more of B. bifidum, B. breve, B. animalis, B. longum, L. acidophilus, L. paracasei, L. casei, L. rhamnosus, Lc. Lactis and B. subtilis. Indeed, phage therapy has been proposed as an alternative to antibiotics to take advantage of the lytic (if not also the lysogenic) properties of phage against bacteria and treat bacterial infections. See, e.g., Borysowski, J. et al, international Journal of Infectious Diseases (2008) 12, 466-471. In addition, phage have immunomodulatory and anti-inflammatory properties (inhibition of NF kappa 6 activation, potentially useful for maintaining homeostasis of the human immune system and have shown activity against viral Infections of mammalian and cells in vitro as well as antifungal activity. Gorski, A et al, Frontiers in microbiology, 2019, 9: 3306, doi: 10.3389/fmicb.2018.03306. The present dietary supplements may contain one or more phage as an optional component together with the combination of trace mineral complex/theanine/peptides with or without one or more additional optional components discussed herein. The phage can be selected from a wide variety of phage as they are used for their prebiotic effect. Examples of bacteriophages that can be used in the present dietary supplements include one or more phages and preferably two or more phages, such as for example a combination of LH01-Myoviridae, LL5-Siphoviridae, T4D-Myoviridae, and LL12-Myoviridae. See generally, Vitetta, L et al, Front Immunol. 2018; 9: 2240. doi: 10.3389/fimmu.2018.02240.

Other phages that can be used include phages disclosed in: Marcó M8, Moineau S, Quiberoni A. Bacteriophages and dairy fermentations. Bacteriophage. 2012; 2(3):149-158. doi:10.4161/bact.21868; Ackermann, H. W., 5500 Phages examined in the electron microscope Brief Review. Arch Virol (2007) 152: 227-243 DOI 10.1007/s00705-006.0849-1; Lehman S M. Mearns G, Rankin D, et al. Design and Preclinical Development of a Phage Product for the Treatment of Antibiotic-Resistant Staphylococcus aureus Infections. Viruses. 2019; 11(1):88. Published 2019 Jan. 21. doi:10.3390/v11010088; Muntasir Alam, Marufa Zerin Akhter, Mahmuda Yasmin, Chowdhury Rafiqul Ahsan, Jamalun Nessa Local bacteriophage isolates showed anti-Escherichia coli O157:H7 potency in an experimental ligated rabbit ileal loop model. Canadian Journal of Microbiology, 2011, 57(5): 408-415, https://doi.org/10.1139/w11-020: and Fortier L C, Sekulovic O. Importance of prophages to evolution and virulence of bacterial pathogens. Virulence. 2013; 4(5):354-365. doi:10.4161/viru.24498.

The daily amount of phage supplementation in the present dietary supplements is generally within the range of about 2 to about 200 mg, typically within the range of about 5 to about 100 mg and more specifically within the range of about 10 to about 50 mg.

Enzyme Components

Human digestive enzymes are capable of degrading only a few glycosidic linkages present in a subset of carbohydrates, including starch polysaccharides. At least one of the following enzymes, and preferably combinations of two or more of them, may advantageously be combined in the present formulation as an optional but beneficial component to the dietary supplements of the present disclosure in amounts effective to assist the subject's digestion. Enzyme dosages may vary and depend on the activity of each enzyme as well as on the formulation as certain excipients affect activity and enzymes may advantageously be enteric coated if desired to be active in the small intestine. Commercially available enzymes are sold with activity values given. Ianiro, G. et al, Current Drug Metabolism, 2016, 17, 187-193.

Exemplary enzymes and exemplary amount/activity ranges for such enzyme(s) used in the dietary supplements of the present disclosure are as follows (per gram as indicated):

Amylolytic Activity (“Amylase”)
The total amylase activity will be in the range of about 100 to about 40,000 units, typically 2,000 to 20,000 units and more specifically about 2500 to about 10,000 DU (dextrinizing units) per gram of total amylases.
   Alpha (fungal) amylase: generally, from about 400 DU/g to about 15,000 DU/g; typically, from about 1000 DU/g to about 12,000 DU/g and more specifically from about 2000 DU to about 7500 DU/g);
   Amylase: generally, from about 500 to about 10,000 SKB, typically from about 1,000 to about 7500 SKB and more specifically from about 3,000 to about 6,000 SKB;
   Amyloglucosidase: generally, from about 2.5 to about 5000 AG (amyloglycosidase units), typically from about 500 to about 2500 AG/g, and more specifically from about 750 to about 2000 AG/g.

Proteolytic Activity
Protease: generally, from about 2500 to 120,000 HUT/g (protease units on an L-tyrosine basis); typically, from about 4,000 to about 80,000 HUT/g and more specifically from about 5, 000 to about 50,000 HUT/g.

Cellulolytic Activity
Cellulase: generally, from about 1000 to about 100,000 CU/g, typically from about 10000 to about 80,000 CU/g, and more specifically from about 20000 to about 75000 CU/g (cellulase units)
Lactase:
Beta-D-galactoside galactohydrolase: generally, from about 20 to about 6000 ALU/g, typically, from about 75 to about 6000 ALU/g, and more specifically from about 100 to about 6000 ALU/g (acid lactase units)

Maltase
Maltase: generally, from about 10 to about 1000 DP/g (degrees diastatic power), typically, from about 100 to about 700 DP/g, and more specifically, from about 200 to about 600 DP/g

Lipolytic Activity
Lipase: generally, from about 50 to about 10,000 FIP/g, typically, from about 100 to about 7500 FIP/g, and more specifically, from about 1,000 to about 3000 FIP/g (fungal lipase international units)

Bromelain
Bromelain: generally, from about 100 TO 1,200 GDU/g (gelatin digesting units), typically, from about 200 to about 1,000 GDU, and more specifically, from about 300 TO 700 GDU/g.

Papain
Papain: generally, from about 2000 to about 5 million PU/g (FCC or USP papain units), typically, from about 1,000,000 to about 4,000.000 PU/g, and more specifically, from about 1,500,000 to 3,000,000 PU/g.

The foregoing enzymes are commercially available. The total amount of enzymes is within the typical range from 25 to 600 mg per daily dose wherein the individual enzymes can be present in equal or varying mg amounts. An exemplary amount for an individual enzyme is 5 mg. The absolute amount of each enzyme used depends on its specific activity, so the amounts given here are a guidance.

Fermented Food Component

Fermented foods have attracted interest because of their anti-oxidant, anti-hypertensive and probiotic activity as well as for providing vitamin supplementation, improving digestibility of food and alleviating lactose intolerance. See, e.g., Melini, F. et al Nutrients 2019, 11, 1189; doi:10.3390/nu11051189. The present dietary supplement may also contain a fermented food component typically comprising one or more and preferably two or more of the following fermented plant foods (fruits, seeds, nuts and other fermented plant products): orange, pineapple, banana, apple, papaya, guava, melon, brown rice, oat, corn, barley, pea, jalo bean, roxinho bean, black sesame seed, millet, plum, adzuki bean, soy bean, carrot, rye grain, black bean, lentil bean, avocado, acerola, lemon, pear, tomato, red grape, mango, watermelon, pumpkin, sweet potato, chick pea, carambola, cashew nut, brazil nut, kiwi, cassava root, green bell pepper, sugar beet, collard greens (leaves), cabbage, passion fruit, chicory (leaves), lemon grass (leaves), lotus root, turnip (root), seaweed (kombu), mate (leaves), cinnamon (root), anise (fruit), clove (leaves), ginger (root), zedoary (root).

A fermented food component may advantageously be added to the dietary supplements of the present disclosure containing a combination of a trace mineral complex and theanine and optionally also one or more bioactive peptides. Such combinations may be further augmented by one or more of the other optional components disclosed here.

The amount of fermented foods if any to be included in the dietary supplements of the present disclosure can vary broadly. Exemplary ranges include a broader range from about 10 mg to about 5 g, more usually from about 50 mg to about 3 grams and more specifically from about 100 mg to about 2 g.

Excipients and Dosage Forms

The dietary supplements of the present disclosure may comprise a solid form or a liquid form, such as a suspension. Solid forms include without limitation a powder, a tablet, a capsule, a caplet, a sachet, or an encapsulated liquid. Advantageously, the composition is in the form of free powder or a capsule. Such dosage forms can be prepared by compounding, suspending and capsule filling techniques known in the art.

Excipient that may be optionally incorporated in the dietary supplements of the present disclosure include fillers, flow agents, colorants, flavoring agents, binders, disintegrants, solubilization agents, viscosity modifiers, surfactants, preservatives and combinations thereof. The excipients together may typically constitute from about 1 to about 10 weight percent of the composition. The excipients are preferably food grade.

Examples of fillers include microcrystalline cellulose, ethyl cellulose, lactose (hydrated or anhydrous), vegetable glycerin.

Examples of binders include cellulosic binders (for example CompaceCell available from BioGrund (www.biogrund.com)), maize starch, Methocel™ cellulose ethers, etc.

Examples of colorants: sodium erythrosine, zinober, ferric oxide red, ferrous oxide yellow, malachite, azurite, titanium oxide, limestone, carbon, sooth, beta carotene, turmeric, other clean label colorants.

Examples of flavoring agents: sugar, sucralose, stevia-based sweeteners, sugar alcohols, natural flavoring agents such as citric acid, malic acid etc.

Examples of disintegrants: croscarmellose sodium, citric acid.

Examples of preservatives: citric acid salts such as sodium citrate, sodium sorbate, sorbic acid, citric acid, benzoic acid and benzoates, lactic acid and lactates, sulfites and ascorbates.

Examples of flow agents include silica (which also acts as a desiccant), colloidal silica, magnesium silicate, metallic salts of stearic acid such as magnesium stearate, hydrated magnesium stearate, calcium stearate, and zinc stearate; metallic salts of lauric acid, myristic acid, palmitic acid, glyceride esters such as glyceryl monostearate, glyceryl tribehenate, and glyceryl dibehenate) and sugar esters (sorbitan monostearate and sucrose monopalmitate) and preferably rice bran concentrate, rice extract, rice hulls, gum arabic sunflower oil, vegetable cellulose, microcrystalline cellulose. Generally, the flow agents will comprise about 0.25% to about 5.0%, w/w of the total composition.

Examples of surfactants: lecithin, lipoproteins, quillaja extract, bile salts.

The capsule material may be made of cellulose, hydroxypropylmethyl cellulose (HPMC), gelatin, tapioca starch polysaccharide.

The present dietary supplements can be tested for activity as illustrated in the following nonlimiting examples. The reagents and techniques described below can be applied to any dietary supplement or components thereof and combinations of components disclosed here. Moreover, discussions about experimental rationale, background information, alternative ways of testing a substance constitute general discussions and such are not confined to the environment of a particular experiment but apply to the present disclosure as a whole.

As supported by the experiments, discussions and conclusions contained below (which are broader than the precise experimental system used) the present supplements can and do Influence a variety of features and parameters of the human intestine. The supplements of the present disclosure, whether comprising one component or a combination of components have been shown to be active in favorably influencing one or more such features and parameters. For example, the trace mineral complex, used alone was active in increasing levels of one or more tight junction proteins and one or more of microvilli proteins, which are lumen surface area indicators. The complex was also active in increasing TEER, also a measure of strength (‘tightness”) of the epithelial barrier. L-theanine alone was very active in fortifying barrier function, and one or more tight junction proteins and one or more surface area Indicators. Combinations of L-theanine with peptide with and without the trace mineral complex and also the full product had broader activities towards improving the barrier (including notably increasing claudin-1 and occludin, increasing mucin-3, decreasing claudin-2, a negative indicator of barrier strength, and increasing TEER, by protecting the epithelial barrier from inflammation damage) and increasing one or more of the surface area indicators as well as increasing the population of at least one phylum and species tested. Lastly, the full product, which contained a combination of all six individual components showed activity in these areas despite its containing components which were shown to adversely impact some of the desirable features and parameters involved. Lastly, the polyphenol preparation used alone showed activities for which it was not previously known, as described below and claimed.

### EXPERIMENTAL

### Example 1: Short-Term Colonic Simulation Experiments

A validated (Van der Abbeele, P. et al, The ISME Journal (2013) 7: 94-961) short-term (ST) human colonic (luminal (L-) and mucosal (M-)) simulation system (SHIME® ProDigest, Gent, Belgium) available commercially on a fee-for-service basis (any other validated in vitro model of the colon, TNO intestinal model, also available on a fee for service basis from TNO and Triskelion in the Netherlands, may have been used instead), was used in this in vitro study to investigate the effects of components and combinations of components of the present dietary supplement on the human microbiome. The one-vessel, batch, SHIME® colonic simulation module was employed. Each vessel is independent. It simulates the human colon (proximal large intestine) and is inoculated with fecal microbiome from a single healthy adult donor (21-45 yrs., average body mass index, typical western diet) who had been antibiotics-free for at least one year. One reactor is run as a negative control with no component of the present dietary supplement added to establish a baseline and, where applicable, one reactor is run with a positive control. A different reactor vessel was used for each test product, for 8 test products, one negative control and one positive control, I.e., for a total of 33 vessels (33 Incubations) including the control (3 different amounts of each test product and positive control were used and the negative control was run in triplicate). The vessels were kept under volume, pH and temperature control and under a nitrogen atmosphere to simulate anaerobic biologic conditions. The volume was 600 ml, the temperature was maintained at 37° C. and, the pH was maintained at physiologic levels by using 0.5 M HCl and 0.5 M NaOH; the consumption of these ingredients was recorded. Mild agitation was provided by agitating at 90 rpm. The defined growth medium (buffer and minimal nutrition medium) is commercially available from ProDigest, supra. According to ProDigest literature provided in connection with this service, the medium contains (in g/L) arabinogalactan (1.2), pectin (2.0), xylan (0.5), resistant starch (4.0), glucose (0.4), yeast extract (3.0), peptone (1.0), mucin (3.0), and cysteine (0.5). It is thus relatively glucose-depleted. The defined medium was adjusted to pH 6.5 prior to autoclaving, then autoclaved and used.

Prebiotic effects of the various test substances were also evaluated using mucin beads that simulate the mucin-coated environment of the colon. Mucin-coated carriers were provided as mucin bead bags (5 beads per bag, one bag per reactor) prepared by dipping plastic, hollowed bead carriers (DI, 5 mm; ProDigest) into a mucin-agar solution. The Mucin-Agar solution was prepared by boiling 1% bacterial agar in autoclaved MilliQ water three times, to dissolve 5% type II porcine mucin (Sigma-Aldrich). The filled carriers were allowed to solidify under laminar flow in a biosafety cabinet at room temperature and stored at −4° C. until use. The mucin beads were used to simulate the intestinal mucosa, providing a surface for bacteria to adhere and grow. For additional details of this procedure, see Van den Abbeele, P. et al, 2012, Microb Biotechnol. 5(1):106-15 and Van den Abbeele, P. et al, 2013, supra.

Fecal samples were tested for pathogens. Fresh fecal inocula were prepared as follows: 7.5% fecal suspensions from a single healthy adult donor were prepared in anaerobic PBS, stomachered, centrifuged at mild speed, supernatant collected and homogenized before administration. (Frozen samples (−80° C.) could have been used instead.) The supernatant was used as the inoculum at 7 ml per reactor. The reactor vessels were incubated for 48 hours. No stabilization of the culture was needed in this batch experiment.

Measured quantities of the test materials in proportion to the size of the reactor and nontoxic to the culture in the reactor were added to each reactor at time 0 (start of experiment) as follows: negative control (to establish microbiome baseline), bioactive peptide mixture alone, L-theanine alone, XOS fiber prebiotic alone, nonfiber polyphenol prebiotic alone, polynucleotide mixture alone and trace mineral complex alone, as well as combinations thereof as described in Table 1 below. The test products were each added suspended in 63 ml of carbohydrate-depleted background nutritional medium (and 7 ml of fecal inoculum) without predigesting. The mixture was incubated for 48 hours.

All percentages given here are by weight.

TABLE 1
Overview of product concentrations (in
reactors at start of experiment)

| | Concentration | | | |
|---|---|---|---|---|
| | of individual | | | |
| | components if | Low | Medium | High |
| | 5 g/L of full | dose | dose | dose |
| Test Product | product tested | (g/L) | (g/L) | (g/L) |
| Control | | 0 | 0 | 0 |
| Peptide blend | 0.72 | 0.72 | 3.00 | 5.00 |
| L-theanine | 0.24 | 0.24 | 3.00 | 5.00 |
| Trace mineral | 0.04 | 0.04 | 1.00 | 3.00 |
| complex | | | | |
| XOS Fiber | 2.39 | 2.39 | 3.00 | 5.00 |
| prebiotic | | | | |
| Polynucleotide | 0.04 | 0.04 | 3.00 | 5.00 |
| mixture | | | | |
| Polyphenol | 0.95 | 0.25 | 0.95 | 3.00 |
| blend | | | | |
| Full product | 5.00 | 1.00 | 3.00 | 5.00 |
| (combination | | | | |
| of the 6 | | | | |
| foregoing | | | | |
| components) | | | | |
| Mineral complex + L- | 0.99 | 0.99 | 3.00 | 5.00 |
| theanine + | | | | |
| peptide blend | | | | |
| L-theanine + | 0.95 | 0.95 | 3.00 | 5.00 |
| peptide blend | | | | |
| Inulin (positive | 5.00 | 2.39 | 3.00 | 5.00 |
| control) | | | | |

The composition of each test product was as follows:

The Peptide blend contained the following peptides: pea peptide, rice peptide, mung bean peptide, and bitter melon peptide.

The L-Theanine test product contained 100% L-theanine.

The Nucleotide test product contained 60% nucleotides from S. Cerevisiae and arabic gum.

The trace mineral complex contained trace mineral complex, commercially available.

The Polyphenol test product contained equal amounts of each of the following ingredients.

Apple fruit extract

Cranberry fruit

Purple sweet potato extract

Mango leaf extract

Indian Barberry/berberine

Cocoa seed extract

Quercetin

Avocado fruit extract

Skullcap root extract

Passion flower extract

Blueberry extract

Sea buckhorn fruit

Turmeric

Pomegranate extract

The XOS test product contained about 95% of XOS, commercially available.

The L-theanine+Peptide combination product contained 25% L-theanine and the remainder was the peptide product.

The L-theanine+trace mineral complex+peptide combination product contained 24.2% of the L-theanine product, 3.22% of the trace mineral complex product and the balance (approx. 72.6%) of the peptide product.

The “full product” contained, 17.9% of the peptide test product, 8.55% of the Nucleotide test product, 7.98% of the polyphenol product, 0.57% of the trace mineral complex product, 42.74% of the XOS product and 4.27% of the L-theanine product. The remainder was composed of various vitamins (A. D2, BS), microelements (Zn, Se), fruit and plant powders and extracts (superfoods), enzymes and phage. The enzymes were a major component at Thus, the full product contained substantially diluted amounts of the individual components tested.

The positive control, inulin, was a commercially available product said to contain 100% inulin, a fructan-based polysaccharide product of plant tuber origin.

Samples of luminal fluid were collected at 0 (for control), 6, 24 and 48 hrs. This permitted assessment of metabolite production. Gas pressure was measured after sampling at 0, 6, 24 and 48 hrs. after the start of incubation; pH was also assessed at the same times.
Gas pressure provides a measure of microbial activity and therefore fermentation speed as reflected by the production of gas in the head space of the reactor, which is a closed system. H₂ and CO₂ are the first gasses to be produced upon microbial fermentation; they can subsequently be utilized as substrates for CH₄ production, reducing the gas volume. H, can also be utilized to reduce sulfate to H₂S, resulting from proteolytic fermentation. Scaldaferri et al. (2013) Intestinal gas production and gastrointestinal symptoms: from pathogenesis to clinical Implication. Eur Rev Med Pharmacol Sci 2013; 17 (2 Suppl): 2-10. As a result, N₂, O₂, CO₂, H₂ and CH₄ account for 99% of the volume of intestinal gas. The remaining 1% consists of NH₃, H₂S, volatile amino acids and short chain fatty acids. Avunduk C. (2008). Manual of Gastroenterology Diagnosis and Therapy—Fourth edition. Lippincott Williams & Wilkins. Excessive production of gas is considered as a potentially negative side-effect of increased saccharolytic activity of the intestinal community upon administration of a test product. As different bacterial groups produce different amounts of gasses, the substrate-specific stimulation of certain microbial groups will allow for more or less gas production.
pH reduction also provides a rough measure of the speed of fermentation, which results in acid production, causing the pH to decrease. The degree of acidification is a measure of the Intensity of bacterial metabolism. In general, lower pH is unfavorable to pathogens. Bacterial fermentation of polysaccharides results in the production of acidic fermentation end products, primarily lactic acid and SCFAs, that reduce the colonic pH which in turn Impacts the composition of the microbial communities present in the GI tract. Normal human colonic pH values are between pH 5.5 and 7.5. In vitro fermentation experiments to model the colon reveal that a reduction in pH from 6.5 to 5.5 (being maintained within normal limits) significantly alters the bacterial community. The more acidic conditions better support growth of butyrate-producing Firmicutes, such as Roseburia spp., while reducing the proliferation of the acid-sensitive Bacteroides spp. F. prausnitzii is also a low pH tolerant organism that will “bloom” under certain luminal conditions.

On the other hand, a decrease in the pH has also been reported to affect SCFA production fueled by bioactive peptides. Walker, A. W., et al, APPL. ENVIRON. MICROBIOL. 71(7): 3692-3700 (2005).

Both pH and gas pressure were measured at sampling time.

The luminal samples were centrifuged and processed for DNA extraction, short-chain fatty acid analysis, and ammonium production; at 48 hours a sample was collected to be used for cell-based experiments (see Example 2) (sample centrifuged at 9000 rpm, 5 min and supernatant retained).
Short chain fatty acid (SCFA) analysis: The pattern of SCFA is an assessment of the microbial carbohydrate metabolism (acetate, propionate and butyrate) or protein metabolism (branched SCFA: isobutyrate, isovalerate and isocaproate) and can be compared to typical fermentation patterns for normal GI microbiota as well as with the negative control. Samples were taken at 0, 6, 24 and 48 hrs. (See for example, Liu, et al. Establishing a mucosal gut microbial community in vitro using an artificial simulator. PLoS ONE 13(7): e0197692. 2018). In general, higher SCFA values and in particular higher butyrate values and butyrate portions of SCFA are associated with a healthy intestinal microbiota and a healthy gut. (There is some controversy regarding butyrate and obesity. The majority of the studies shows it provides a protective effect against obesity, especially in high fat diets, it may also improve glucose homeostasis and reduce insulin resistance. However, certain other studies show elevated butyrate levels in obese individuals. The mixed nature of the data may be related to dietary choices, or to other metabolic factors or variations in individual microbial profiles. This is an area where more study is needed, but SCFA production and butyrate levels are shown to be predominately correlated with positive health benefits.)

SCFAs account for 2-10% of the total energy consumption in humans, are the main energy source for large intestinal epithelial cells, and affect the production of mucins (mucus). In addition, SCFAs physiologically influence blood flow to the colon mucous membrane, the absorption of fluids and electrolytes, the autonomic nervous system, and the secretion of gut hormones. A considerable part of the beneficial effect of prebiotics is thought to be due to SCFAs produced by intestinal microbes. Research has shown that the concentration of SCFAs is 70-140 mmol/L in the proximal colon and 20-70 mmol/L in the distal colon. In general, acetate is thought to be more prevalent, followed by propionate and butyrate. Animal experiments have shown that the total amount of SCFAs may be approximately 400-600 mmol/day when 60 g/day of undigested carbohydrates reach the colon. (Ohira, H. et al, J Atheroscler Thromb. 2017 Jul. 1; 24(7): 660-672; doi: 10.5551/jat.RV17006). It is anticipated that the amount of butyrate will increase (compared to the butyrate produced when nondigestible carbohydrate prebiotic is used alone) when nondigestible carbohydrate prebiotic is provided to the test system in addition to two (trace mineral complex/L-theanine) or three (trace mineral complex/L-theanine/peptides) core ingredients of the present dietary supplements, even when one or more of the core ingredients are provided in suboptimal or even subthreshold amounts. An increase in SCFA production and/or production efficiency will translate into amelioration of the luminal microbial environment and restoration of balance of the intestinal immune system.

Although it is the least abundant SCFA produced (˜60% acetate, 25% propionate, and 15% butyrate in humans) butyrate is the major energy source for colonocytes. Thus, it is desirable to increase butyrate while maintaining it of course well below toxic levels (if any). For example, in the past, 100 mM butyrate by rectal administration was commonly used in clinical practice, which is comparable with physiologic concentrations in the colon of humans after the consumption of a high-fiber diet. No toxicity was observed.
Lactate analysis: The human intestine harbors both lactate-producing and lactate-utilizing bacteria. Lactate is produced by lactic acid bacteria and decreases the pH of the environment (within the normal pH range which in the colon is 5.5 to 7.5), acting also as an antimicrobial agent and decreasing some pathogens in the gut. Protonated lactic acid can penetrate the microbial cell after which it dissociates and releases protons within the cell, resulting in acidification and microbial cell death. It can also be rapidly converted to acetate, butyrate, and propionate by other microorganisms. Samples for lactate analysis were analyzed after 0, 6, 24 and 48 h of incubation.
Ammonium analysis: Ammonium is a product of proteolytic degradation which results in the production of potentially toxic or carcinogenic compounds such as p-cresol and p-phenol. It can be used as an indirect marker for low substrate availability. Since it is only produced towards the end of the incubation, it is measured only at 24 and 48 hrs.
Microbiome composition: The microbiome composition from the simulator of the proximal large intestine was analyzed by quantitative PCR (qPCR) and compared to the internal negative control as well as to accepted norms for certain microbial species. At the taxonomic phyla level, a healthy microbiota in adult humans is principally composed of Firmicutes and Bacteroidetes, which together represent approximately 70% of the total microbiota; Proteobacteria, Verrucomicrobia, Actinobacteria, Fusobacteria, and Cyanobacteria can also be found, although at lower percentages. Obligate anaerobes dominate and exceed by two logs the facultative anaerobes and by three logs the aerobes. At the taxonomic level of species, the gut microbiota composition changes from individual to individual and is comparable to a fingerprint. Gagliardi, A. et al, Int. J. Environ. Res. Public Health, 2018, 15, 1679; doi:10.3390/ijerph15081679.

Both indicators of the mucosal microbiota and of the luminal microbiota were examined. Both are essential to optimal gut health. The structural elements of the gastrointestinal tract help these organisms play a very different role in gastrointestinal and human health⋅ besides just keeping pathogens out of the lamina propria. The amount and type of bacteria associated with the mucus layer also affects the production of mucin and other structural components of the intestinal barrier beyond excluding pathogens from colonizing the mucus layer.

The 165 rRNA gene consists of variable and conserved regions, spread over the gene. Due to their key role in protein expression, the conserved regions are characterized by very low evolutionary rates. Any mutations that occurred in these regions during evolution have inevitably led to the death of the corresponding organism. Conservation of these 165 rRNA gene sequences allows the design of universal primers targeting the complete bacterial pool in a sample. Next to these conserved gene regions, the 16S rRNA gene also contains nine variable regions (V1-V9), which are characterized by a much higher evolutionary rate. These gene regions are typically less essential for the survival of the organism, which is why any mutations in these regions did not lead to death of the organism during evolution. Considering their higher evolutionary rates, these gene regions are typically used to distinguish between different taxonomic groups of bacteria.

Samples for the qPCR analysis of the microbial community composition were collected (separately for each vessel) and analyzed using primers targeted to specific bacterial sequences (i.e., 165 rRNA genes) by methods known by those skilled in the art. See, e.g., Possemiers, S. et al, J. Agric. Food Chem. 2013, 61, 9380-9392 dx.doi.org/10.1021/jf402137r | at Table 2 of this reference. The following groups of micro-organisms were monitored as indicator genera or species of intestinal microbial health and diversity: Firmicutes (F) and Bacteroidetes (B), the two most dominant bacterial phyla with the aim of detecting a favorable relative increase in the ratio of Bacteroidetes to Firmicutes (while Firmicutes may continue to be more plentiful than Bacteroidetes). An increase in the ratio of B/F is associated with leaner body mass and greater metabolic efficiency. Bifidobacteria and lactobacilli, two specific groups which are typically of Interest were also monitored with the experiment aiming to detect a relative increase in Bifidobacteria. Bifidobacteria, through their generation of lactate, support other bacterial species that consume lactate and in turn produce butyrate.

The truly anaerobic (as opposed to facultative and/or spore former) Akkermansia spp. was similarly monitored. A. muciniphila in particular is not generally available as a probiotic. Its numbers can be affected through diet but this requires a healthy gut structure and specifically a healthy mucin layer which this microorganism colonizes and feeds on. Relative increases in this Akkermansia are beneficial in that predominance of these bacteria is negatively associated with obesity, diabetes, cardiometabolic disease and low-grade inflammation, and positively associated with improvements in glucose homeostasis, blood lipid levels and body composition after caloric restriction. It is anticipated that through the use of the present dietary supplement there will be relative increases to A. muciniphila which could be attributed to increased butyrate levels. A. muciniphila is a mucus degrading bacteria and colonize the outer mucous layer. Other bacteria that are found in the mucus layer do not have the ability degrade mucin. They depend on the mucolytic activity of other microbiota members to colonize the mucus layer. Degradation of the mucin protein backbone and the release of sugars from the glycan chains, provide carbon and nitrogen for the bacteria that do not produce these enzymes. Butyrate-producers, such as Faecalibacterium prausnitzii, are examples of species that do not have the ability to degrade mucus, but are commonly found in the mucus layer. Since butyrate is a source of energy for the colonocytes, the production of butyrate by these organisms nearby the epithelium layer is a useful benefit for the host. An actual syntrophy between A. muciniphila and various butyrate producers was shown in vitro. The mucolytic activity of A. muciniphila lead to accumulation of acetate and mono-saccharides in the medium. These were fermented to butyrate by the cross feeding butyrogens such as F. prausnitzii. Ottman N., et al, Action and function of Akkermansia muciniphila in microbiome ecology, health and disease. Best Pract Res Clin Gastroenterol. 2017 December; 31(6):637-642. doi: 10.1016/j.bpg.2017.10.001. Epub 2017 Oct. 13. Thus, these bacteria are an important indicator of gut health.

Lastly, the commensal true anaerobic bacterium Faecalibacterium prausnitzii was monitored. Characterized as a commensal bacterium, this species has been shown to have an impact on the physiology and health of the host in that its depletion is associated with several intestinal disorders, and more consistently in Crohn's disease. (Martin, R. et al, 2017). An increase in the numbers of this bacterium to comprise 3 to 5% of the human fecal microbiota is considered Important to achieving a homeostatic balance among gut microorganisms. These are also mucosal associated bacteria that act as “peacekeepers” in the gut and help maintain critical balance. The establishment of these bacteria in the GI tract is influenced by other commensal species, redox mediators, oxygen concentration (F. prausnitzii is oxygen sensitive), mucus layer as well as bile salt concentrations and pH. As F. prausnitzii is one of the most abundant butyrate-producing species, its beneficial effects have been first attributed to butyrate. Butyrate is involved in the cross-feeding between butyrate producer bacteria and Bifidobacterium sp. which favors the co-existence of bifidobacterial strains with other bifidobacteria and with butyrate-producing colon bacteria in the human colon with the consequent benefit to human health. The anti-inflammatory capacities of F. prausnitzii do not seem to be limited to butyrate. F. prausnitzii-mediated butyrate production is not the only beneficial bacterial effector linked to this species in colitis models. Martin R, Bermudez-Humar\dot{a}n L G, Langella P. Searching for the Bacterial Effector: The Example of the Multi-Skilled Commensal Bacterium Faecalibacterium prausnitzii. Front Microbiol. 2018; 9:346. Published 2018 Mar. 6. doi:10.3389/fmicb.2018.00346. Thus, the Importance of this indicator species should not be understated.

Mucin bead bags were removed at 48 h and washed in anaerobic PBS to wash off luminal bacteria; mucin was homogenized for DNA extraction. qPCR of the DNA fraction permits to identify which of the foregoing bacterial groups, if any, establish and thrive on the mucin layer.

The gut is a remarkable organ where nutrients are digested and absorbed. It is inhabited by diverse microbes that play an Important role in maintaining physiological homeostasis of the gastrointestinal system. The gastrointestinal mucosal barrier mainly consists of epithelium, immune cells and resident microbiota. Mucosal barrier not only protects the gastrointestinal tract (GIT) against bacteria, other pathogenic microbes and other toxic substances, but also facilitates the passage of food in the gut and allows the low molecular weight nutrients to pass the barrier of the epithelial cells. Gastrointestinal mucosal surfaces are usually covered with a thick layer of mucus, which is synthesized by goblet cells in the epithelium and forms a protective physical barrier. The colon has two layers of mucus with distinct physical properties: an inner firmly adherent mucus layer (which does not ordinarily host bacteria) and an outer less dense loosely adherent mucus layer. In contrast to the colon, the small intestine has only one layer of mucus. (He, J. et al, Current Protein and Peptide Science ⋅ May 2018; DOI: 10.2174/1389203719666180514152406).

In addition to providing habitat, mucus can provide energy sources for some commensal microbes through mucin degradation, while gut microorganisms also influence mucin gene expression, glycosylation, and secretion (9). Furthermore, once commensal microbes are attached to host mucus, they keep pathogenic organisms from occupying mucus empty niches (10). (Sicard, J. F.; Le Bihan, G.; Vogeleer, P.; Jacques, M.; Harel, J. Interactions of Intestinal Bacteria with Components of the Intestinal Mucus. Front. Cell Infect. Microbiol. 2017, 7, 387.

Another feature of the colon mucous layer is its rapid turnover time. A recent mouse study has revealed that the inner mucous layer is fully renewed within 1 h, while the gut epithelium tissue is self-renewed within 4-5 days. Hence, it is clear that mucus forms a remarkable and sensitive niche for microbial life, in which microbes are challenged to adapt to the biochemical dynamics determining mucous viscoelasticity and to persist within a continuously renewed environment. Johansson M E. Fast renewal of the distal colonic mucus layers by the surface goblet cells as measured by in vivo labeling of mucin glycoproteins. PLoS One. 2012; 7(7):e41009. doi:10.1371/journal.pone.0041009.

The results of the foregoing experiments were as follows:

A) Microbial Metabolic Activity
pH decrease: Monitoring the pH during a colonic incubation provides a good indication of the production of SCFA, lactate and ammonium (NH4+). In general, a pH drop is observed during the first 24 h of incubation due to the formation of SCFA/lactate. This pH drop is often followed by a pH increase during the last 24 h of incubation due to proteolytic fermentation, which results in the production of amongst others NH4+, and due to conversion of stronger acids into weaker acids through cross-feeding (for instance acetate/lactate-to-propionate/butyrate conversion).

XOS, the full product, the positive control inulin and the high dose of the polyphenol prebiotic blend resulted in stronger pH decreases than the blank during the first 6 h of incubation. This indicates that product fermentation started at an early stage of the incubation (Table 2 and Table 3). Higher product doses resulted in stronger pH-decreases. The strongest initial pH decreases were obtained with XOS. Interestingly, these increases were markedly stronger than the ones noted for the positive control inulin.

In general, after the initial pH drop, the pH tended to remain stable or moderately increased between 6 and 24 h for all treatments, except for the high inulin dose, where an additional pH-decrease was observed. As mentioned above, the pH-increase can be due to production of ammonium (indicative of substrate depletion), or conversion of stronger acids (acetate and lactate) into weaker acids (propionate and butyrate) due to cross-feeding. The strongest pH increases were obtained with the low and medium doses of the full product and with the high L-theanine dose.

TABLE 2
Absolute pH at different time points of fermentation (0, 6,
24 and 48 h of incubation) of various test products by gut
microbiota of a healthy adult subject. Negative control:
blank; positive control: inulin. (0-6 h, 6-24 h, 24-48 hr)

| | | pH absolute | | | |
|---|---|---|---|---|---|
| | | 0 h | 6 h | 24 h | 48 h |
| Blank | | 6.49 | 6.38 | 6.50 | 6.47 |
| Peptide blend | Low | 6.47 | 6.41 | 6.51 | 6.45 |
| | Medium | 6.47 | 6.37 | 6.51 | 6.48 |
| | High | 6.48 | 6.36 | 6.52 | 6.48 |
| L-theanine | Low | 6.50 | 6.40 | 6.51 | 6.49 |
| | Medium | 6.49 | 6.39 | 6.54 | 6.49 |
| | High | 6.49 | 6.38 | 6.57 | 6.50 |
| Trace Mineral | Low | 6.51 | 6.40 | 6.51 | 6.49 |
| Complex | Medium | 6.50 | 6.41 | 6.49 | 6.45 |
| | High | 6.50 | 6.40 | 6.50 | 6.45 |
| XOS prebiotic | Low | 6.50 | 5.96 | 6.11 | 6.11 |
| | Medium | 6.49 | 5.85 | 5.99 | 5.97 |
| | High | 6.48 | 5.41 | 5.55 | 5.58 |
| Nucleotides | Low | 6.49 | 6.42 | 6.51 | 6.45 |
| | Medium | 6.46 | 6.36 | 6.45 | 6.41 |
| | High | 6.42 | 6.33 | 6.34 | 6.38 |
| Polyphenol prebiotic | Low | 6.51 | 6.41 | 6.49 | 6.50 |
| blend | Medium | 6.49 | 6.38 | 6.46 | 6.43 |
| | High | 6.46 | 6.27 | 6.32 | 6.29 |
| Full product | Low | 6.51 | 6.36 | 6.57 | 6.43 |
| | Medium | 6.48 | 6.17 | 6.39 | 6.27 |
| | High | 6.47 | 6.07 | 6.18 | 6.09 |
| Trace Mineral Complex + | Low | 6.53 | 6.46 | 6.61 | 6.57 |
| L-theanine + | Medium | 6.51 | 6.45 | 6.61 | 6.50 |
| peptides | High | 6.51 | 6.44 | 6.61 | 6.49 |
| L-theanine + peptides | Low | 6.52 | 6.46 | 6.57 | 6.56 |
| | Medium | 6.51 | 6.44 | 6.57 | 6.49 |
| | High | 6.51 | 6.43 | 6.58 | 6.49 |
| Inulin | Low | 6.53 | 6.19 | 6.26 | 6.26 |
| | Medium | 6.52 | 6.14 | 6.15 | 6.08 |
| | High | 6.52 | 6.06 | 5.86 | 5.79 |

TABLE 3
pH change measured at different timepoints in the fermentation
of various test products by the gut microbiota of a healthy
adult subject. Negative control: blank; positive control:
inulin. (0-6 h, 6-24 h, 24-48 hr)

| | | pH change | | |
|---|---|---|---|---|
| | | D 0-6 h | D 6-24 h | D 24-48 h |
| Blank | | −0.11 | 0.12 | −0.03 |
| Peptide blend | Low | −0.06 | 0.10 | −0.06 |
| | Medium | −0.10 | 0.14 | −0.03 |
| | High | −0.12 | 0.16 | −0.04 |
| L-theanine | Low | −0.10 | 0.11 | −0.02 |
| | Medium | −0.10 | 0.15 | −0.05 |
| | High | −0.11 | 0.19 | −0.07 |
| Trace Mineral | Low | −0.11 | 0.11 | −0.02 |
| Complex | Medium | −0.09 | 0.08 | −0.04 |
| | High | −0.10 | 0.10 | −0.05 |
| XOS prebiotic | Low | −0.54 | 0.15 | 0.00 |
| | Medium | −0.64 | 0.14 | −0.02 |
| | High | −1.07 | 0.14 | 0.03 |
| Nucleotides | Low | −0.07 | 0.09 | −0.06 |
| | Medium | −0.10 | 0.09 | −0.04 |
| | High | −0.09 | 0.01 | 0.04 |
| Polyphenol prebiotic | Low | −0.10 | 0.08 | 0.01 |
| blend | Medium | −0.11 | 0.08 | −0.03 |
| | High | −0.19 | 0.05 | −0.03 |
| Full product | Low | −0.15 | 0.21 | −0.14 |
| | Medium | −0.31 | 0.22 | −0.12 |
| | High | −0.40 | 0.11 | −0.09 |
| Trace Mineral Complex + | Low | −0.07 | 0.15 | −0.04 |
| L-theanine + | Medium | −0.06 | 0.16 | −0.11 |
| peptides | High | −0.07 | 0.17 | −0.12 |
| L-theanine + peptides | Low | −0.06 | 0.11 | −0.01 |
| | Medium | −0.07 | 0.13 | −0.08 |
| | High | −0.08 | 0.15 | −0.09 |
| Inulin | Low | −0.34 | 0.07 | 0.00 |
| | Medium | −0.38 | 0.01 | −0.07 |
| | High | −0.46 | −0.20 | −0.07 |

TABLE 4
Gas production upon fermentation of various test products by
the gut microbiota of a healthy adult subject. Negative control:
blank; positive control: inulin. (0-6 h, 6-24 h, 24-48 hr)

Gas production

| | | D 0-6 h | D 6-24 h | D 24-48 h |
|---|---|---|---|---|
| Blank | | 9.0 | 12.7 | 3.1 |
| Peptide blend | Low | 9.7 | 14.4 | 4.0 |
| | Medium | 11.8 | 23.7 | 4.4 |
| | High | 12.8 | 23.7 | 5.0 |
| L-theanine | Low | 9.2 | 14.5 | 4.6 |
| | Medium | 8.7 | 26.5 | 4.4 |
| | High | 8.6 | 32.5 | 5.1 |
| Trace Mineral | Low | 9.1 | 12.3 | 4.7 |
| Complex | Medium | 10.8 | 13.2 | 4.8 |
| | High | 9.2 | 12.9 | 4.8 |
| XOS prebiotic | Low | 18.2 | 18.4 | 4.5 |
| | Medium | 22.4 | 20.7 | 5.4 |
| | High | 26.0 | 7.9 | 13.4 |
| Nucleotides | Low | 9.6 | 13.3 | 5.0 |
| | Medium | 13.2 | 24.5 | 9.6 |
| | High | 14.3 | 30.6 | 13.1 |
| Polyphenol | Low | 9.8 | 13.4 | 4.8 |
| prebiotic blend | Medium | 11.4 | 14.8 | 5.8 |
| | High | 11.6 | 19.0 | 5.6 |
| Full product | Low | 13.5 | 7.2 | 5.8 |
| | Medium | 17.3 | 18.4 | 6.0 |
| | High | 17.9 | 28.3 | 6.6 |
| Trace Mineral | Low | 12.9 | 16.4 | 5.3 |
| Complex + L- | Medium | 11.6 | 24.0 | 6.1 |
| theanine + | High | 12.8 | 7.9 | 6.0 |
| peptides | | | | |
| L-theanine + | Low | 12.5 | 15.7 | 5.4 |
| peptides | Medium | 11.8 | 25.4 | 6.1 |
| | High | 13.5 | 23.3 | 5.7 |
| Inulin | Low | 14.9 | 23.6 | 3.6 |
| | Medium | 14.6 | 30.1 | 3.9 |
| | High | 15.9 | 49.2 | 6.3 |

B) Gas Production

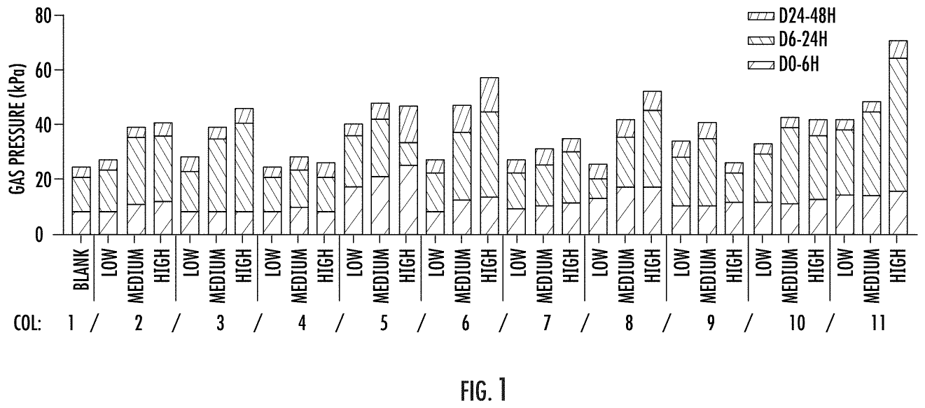

Like pH decrease, gas production is a measure of overall microbial activity, and thus of speed fermentation. Product-related effects on gas production are illustrated in FIG. 1 which is a bar graph of gas production per time interval (0 to 6 h; 6 to 24 h and 24 to 48 h) against test product concentration. In tabular form, the results are shown in Table 4.

The test products XOS, combination of L-theanine+Peptides and inulin stimulated gas production in all three concentrations. The test products peptide blend, L-theanine, nucleotides, polyphenol prebiotic blend and the full product stimulated gas production when administered in medium and high concentrations, indicating that at the low concentrations these test products were depleted. The combination of trace mineral complex+L-theanine+Peptides stimulated gas product when administered in low and medium concentrations. Overall, the highest gas pressures were obtained in the Incubations with the high inulin and nucleotides doses. The highest initial gas pressures (i.e. between 0 and 6 h) were obtained with XOS, indicating rapid product fermentation.

Expectedly, the trace mineral complex resulted in similar gas pressures as the blank, regardless of its concentration. This is consistent with the fact that this complex is mineral and not fermentable. The low concentrations of the test products peptide blend, L-theanine, nucleotides, polyphenol prebiotic blend, full product, and the (outlier) high dose of the combination trace mineral complex+L-theanine+Peptides (the latter during the period 6-24 hours) resulted in similar gas pressures as the blank. Any gas produced in these incubations was thus likely due to proteolytic fermentation of peptides and proteins in the background nutritional medium, rather than being due to product fermentation.

While pH decrease and gas production are both reflective of microbial activity, they are not always correlated with one another, given the fact that different bacterial groups might be responsible for gas production/consumption on the one hand and SCFA/lactate production (which affects pH) on the other hand. The results are also often donor dependent. Thus, outlier values can sometimes be obtained. As discussed below, these products, including the combination of trace mineral complex. L-theanine+peptides showed activity in increasing SCFA in a dose-related fashion.

The test products XOS, full product, positive control inulin and the high dose of the polyphenol prebiotic blend were fermented during the first 6 h of the Incubation, resulting in metabolites that lower the pH of the environment (likely SCFA and lactate). These observations indicate that product fermentation started at an early stage of the incubation. When inulin was used, the pH further decreased between 6 and 24 h in the incubation with the high inulin dose (positive control), suggesting that the substrate at the high concentration was not yet depleted after 6 h. This observation was confirmed by a strong stimulation of gas production within the same timeframe, indicating ongoing product metabolism. The stable or moderately Increasing pH in the other Incubations between 6 and 24 h suggests that products were either depleted after 6 h or that cross-feeding interactions were taking place.

Overall, the strongest initial pH drops and the highest initial gas pressures were obtained with the XOS test product in a dose-related fashion, indicating high fermentability of this test product by the gut microbiota. This is explained by the fact that the XOS product as a fiber prebiotic is a high contributor to fermentation. Furthermore, when used by itself, the XOS was much more concentrated than say the full product of which only a portion was XOS. The other fermentable products stimulated gas production at least at the medium and high concentrations. Theanine alone increased gas production at all concentrations.

B) Changes in Microbial Activity

Short-Chain Fatty Acids

SCFA production results from carbohydrate metabolism in the colon and is associated with various health effects. The most abundantly produced SCFAs include acetate, propionate and butyrate. Acetate can be used as an energy source for the host and as a potential substrate for lipid synthesis in the body; propionate reduces cholesterol and fatty acid synthesis in the liver (beneficial effect on metabolic homeostasis); butyrate is a major energy source for colonocytes and induces differentiation in these cells (which is associated with reduced cancer risk). Positive effects of the investigated substrates on SCFA production therefore include an increase in at least one of acetate, propionate and/or butyrate.

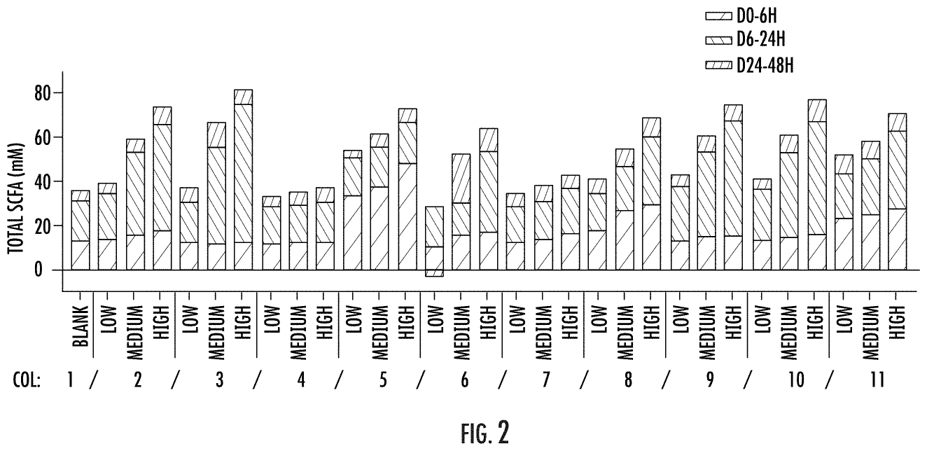

Total SCFA levels in this experiment are reflective of the overall fermentation of test Ingredients. All products, except the nonfermentable trace mineral complex and the polyphenol prebiotic blend (each used alone), yielded significantly higher total SCFA concentrations than the blank in at least one of their tested concentrations (FIG. 2 and Table 6). Even the products that did not increase SCFA significantly above background show a dose-relation, in which SCFA concentrations increased with product dose. XOS, the full product and the positive control inulin stimulated SCFA production already during the first 6 h of the incubation. By the end of the 48 h incubation, the high concentrations of the peptide blend, L-theanine, XOS, the combination of Trace Mineral Complex+L-theanine+peptides and the L-theanine+Peptides mix had yielded the highest SCFA concentrations, exceeding inulin. In fact, when looking at overall SCFA stimulation, L-theanine, the combination test products and the full product also exceeded XOS in their ability to stimulate total SCFA.

Acetate can be produced by many different gut microbes (e.g., Bifidobacterium, Bacteroides) and is a primary metabolite produced from fermentation of prebiotic fibers. All fermentable products except polyphenol blend yielded significantly higher acetate concentrations than the blank in at least one and in all but one case at least two of their tested concentrations (Table 5).

TABLE 5
Acetate and propionate production during different time-intervals upon fermentation,
including blank and positive control, inulin. (0-6 h, 6-24 h, 24-48 hr)

| | | Acetate production | | | Propionate production | | |
|---|---|---|---|---|---|---|---|
| | | D 0-6 | D 6-24 | D 24-48 h | D 0-6 | D 6-24 | D 24-48 h |
| Blank | | 8.8 | 8.3 | 1.2 | 4.3 | 4.3 | 1.3 |
| Peptide blend | Low | 8.2 | 10.1 | 0.8 | 4.5 | 5.4 | 0.8 |
| | Medium | 9.6 | 14.7 | 2.7 | 5.2 | 10.2 | 0.7 |
| | High | 11.0 | 18.0 | 3.5 | 5.7 | 12.8 | 1.3 |
| L-theanine | Low | 8.1 | 9.6 | 1.5 | 4.1 | 4.0 | 1.4 |
| | Medium | 7.6 | 28.6 | 4.6 | 4.0 | 4.0 | 2.5 |
| | High | 7.7 | 42.3 | 1.6 | 4.2 | 4.1 | 1.8 |
| Trace Mineral | Low | 7.3 | 9.3 | 0.2 | 3.9 | 4.3 | 1.0 |
| Complex | Medium | 7.9 | 9.3 | 0.5 | 4.1 | 4.0 | 1.2 |
| | High | 8.1 | 9.7 | 0.9 | 4.3 | 3.9 | 1.4 |
| XOS prebiotic | Low | 22.1 | 8.5 | −0.6 | 10.1 | 4.6 | 1.0 |
| | Medium | 25.5 | 8.0 | 1.4 | 11.7 | 5.4 | 1.2 |
| | High | 34.4 | 7.3 | 2.2 | 13.6 | 7.1 | 0.2 |
| Nucleotides | Low | 7.5 | 9.4 | −3.6 | 4.1 | 3.9 | −0.7 |
| | Medium | 9.7 | 9.1 | 10.5 | 4.9 | 2.9 | 5.8 |
| | High | 10.8 | 23.5 | 3.9 | 5.2 | 7.7 | 2.9 |
| Polyphenol | Low | 8.3 | 8.4 | 1.3 | 4.4 | 3.5 | 1.9 |
| prebiotic | Medium | 8.9 | 9.6 | 0.7 | 4.8 | 3.6 | 2.0 |
| blend | High | 10.5 | 12.5 | 3.1 | 6.0 | 4.7 | 0.6 |
| Full product | Low | 11.8 | 9.5 | 0.4 | 5.7 | 3.6 | 1.7 |
| | Medium | 17.4 | 10.3 | 0.9 | 9.3 | 4.4 | 2.0 |
| | High | 15.5 | 17.6 | 3.9 | 13.9 | 6.7 | 2.0 |
| Trace Mineral | Low | 8.4 | 12.1 | 1.4 | 4.7 | 5.4 | 1.3 |
| Complex + | Medium | 9.3 | 18.5 | 2.7 | 5.0 | 8.2 | 1.7 |
| L-theanine + | High | 10.0 | 24.6 | 2.4 | 5.3 | 11.1 | 1.3 |
| peptides | | | | | | | |
| L-theanine + | Low | 8.6 | 12.4 | −0.6 | 4.8 | 4.9 | 1.2 |
| peptides | Medium | 9.3 | 18.5 | 2.3 | 4.9 | 8.2 | 1.6 |
| | High | 10.1 | 25.1 | 3.0 | 5.2 | 10.8 | 2.0 |
| Inulin | Low | 15.7 | 10.9 | 1.3 | 6.7 | 6.1 | 2.8 |
| | Medium | 17.2 | 13.9 | 0.8 | 7.0 | 7.1 | 2.7 |
| | High | 20.4 | 19.8 | 1.8 | 7.1 | 9.1 | 2.4 |

TABLE 6
Butyrate and total SCFA production during different time-intervals upon fermentation,
including blank and positive control, inulin. (0-6 h, 6-24 h, 24-48 hr)

| | | Butyrate production | | | Total SCFA production | | |
|---|---|---|---|---|---|---|---|
| | | D 0-6 | D 6-24 | D 0-6 | D 6-24 | D 0-6 | D 6-24 |
| Blank | | | 0.6 | 2.6 | 0.6 | 2.6 | 0.6 |
| Peptide blend | Low | 0.6 | 3.3 | 0.6 | 3.3 | 0.6 | 3.3 |
| | Medium | 0.8 | 5.0 | 0.8 | 5.0 | 0.8 | 5.0 |
| | High | 0.9 | 6.7 | 0.9 | 6.7 | 0.9 | 6.7 |
| L-theanine | Low | 0.6 | 2.9 | 0.6 | 2.9 | 0.6 | 2.9 |
| | Medium | 0.5 | 9.9 | 0.5 | 9.9 | 0.5 | 9.9 |
| | High | 0.5 | 14.7 | 0.5 | 14.7 | 0.5 | 14.7 |
| Trace Mineral | Low | 0.5 | 2.3 | 0.5 | 2.3 | 0.5 | 2.3 |
| Complex | Medium | 0.6 | 2.3 | 0.6 | 2.3 | 0.6 | 2.3 |
| | High | 0.6 | 2.1 | 0.6 | 2.1 | 0.6 | 2.1 |
| XOS prebiotic | Low | 0.5 | 4.2 | 0.5 | 4.2 | 0.5 | 4.2 |
| | Medium | 0.5 | 4.2 | 0.5 | 4.2 | 0.5 | 4.2 |
| | High | 0.4 | 3.5 | 0.4 | 3.5 | 0.4 | 3.5 |
| Nucleotides | Low | 0.6 | 2.4 | 0.6 | 2.4 | 0.6 | 2.4 |
| | Medium | 0.7 | 2.0 | 0.7 | 2.0 | 0.7 | 2.0 |
| | High | 0.8 | 3.5 | 0.8 | 3.5 | 0.8 | 3.5 |
| Polyphenol | Low | 0.5 | 2.4 | 0.5 | 2.4 | 0.5 | 2.4 |
| prebiotic | Medium | 0.4 | 2.6 | 0.4 | 2.6 | 0.4 | 2.6 |
| blend | High | 0.3 | 2.6 | 0.3 | 2.6 | 0.3 | 2.6 |
| Full product | Low | 0.5 | 2.8 | 0.5 | 2.8 | 0.5 | 2.8 |
| | Medium | 0.6 | 4.2 | 0.6 | 4.2 | 0.6 | 4.2 |
| | High | 0.4 | 5.3 | 0.4 | 5.3 | 0.4 | 5.3 |
| Trace Mineral | Low | 0.6 | 3.4 | 0.6 | 3.4 | 0.6 | 3.4 |
| Complex + | Medium | 0.7 | 5.8 | 0.7 | 5.8 | 0.7 | 5.8 |
| L-theanine + | | | | | | | |
| peptides | High | 0.8 | 8.0 | 0.8 | 8.0 | 0.8 | 8.0 |
| L-theanine + | Low | 0.7 | 3.3 | 0.7 | 3.3 | 0.7 | 3.3 |
| peptides | Medium | 0.7 | 5.7 | 0.7 | 5.7 | 0.7 | 5.7 |
| | High | 0.7 | 8.0 | 0.7 | 8.0 | 0.7 | 8.0 |
| Inulin | Low | 0.8 | 3.0 | 0.8 | 3.0 | 0.8 | 3.0 |
| | Medium | 0.7 | 4.0 | 0.7 | 4.0 | 0.7 | 4.0 |
| | High | 0.6 | 5.5 | 0.6 | 5.5 | 0.6 | 5.5 |

indicating that product fermentation stimulated acetate production. A dose-relation was observed for the fermentable products, in which acetate concentrations increased with product dose. XOS, the full product and the positive control inulin stimulated acetate production already during the first 6 h of the Incubation and continued to do so throughout the incubation period. By the end of the 48 h Incubation, the highest acetate concentration was obtained with the high L-theanine dose. The medium L-theanine dose and the high doses of XOS, nucleotides, the full product, the combination of trace mineral complex+L-theanine+peptides, the combination of L-theanine+Peptides mix and Inulin also resulted in very high acetate concentrations. A higher net acetate consumption than production rate was observed for the low nucleotides dose during the last 24 h of incubation, evincing consumption of accumulated acetate.

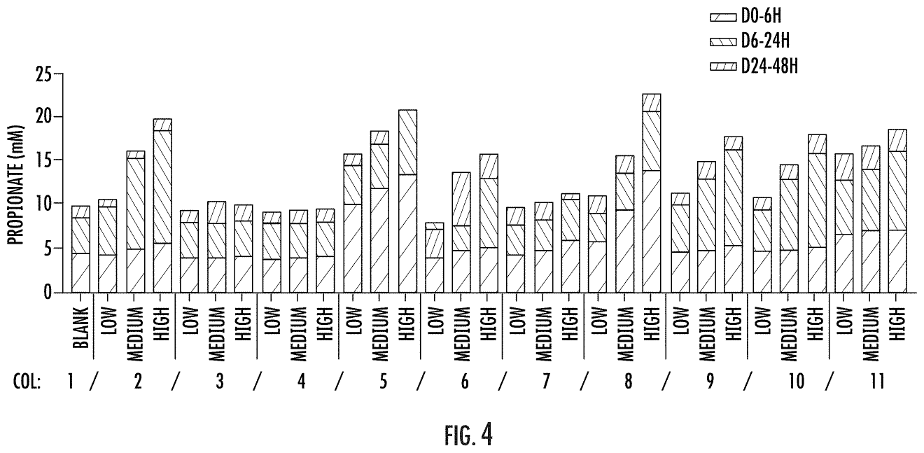

Like acetate, propionate can be produced by a wide range of gut microbes, with the most abundant propionate producers being Bacteroides spp., Akkermansia muciniphila and Veillonellaceae. All products, except L-theanine, trace mineral complex and the Polyphenol prebiotic blend, yielded significantly higher propionate concentrations than the blank in at least one of their tested concentrations (FIG. 4 and Table 5), indicating that product fermentation stimulated propionate production. A dose-relation was observed for these products, in which propionate concentrations increased with product dose. XOS, the full product and the positive control inulin stimulated propionate production already during the first 6 h of the incubation. By the end of the 48 h incubation, the highest propionate concentration was obtained with the high dose of the full product. The high concentrations of the peptide blend and XOS also resulted in very high propionate concentrations.

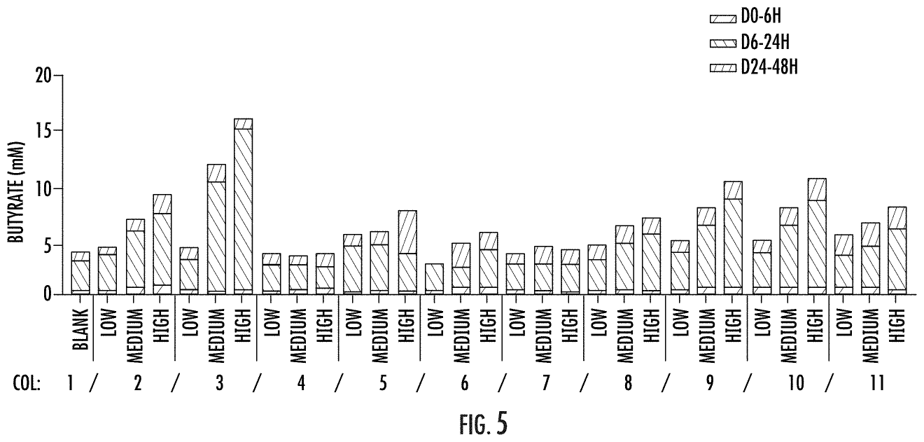

Butyrate is produced by members of the Clostridium clusters IV and XIVa (phylum a Firmicutes). In a process called cross-feeding, these microbes convert acetate and/or lactate (along with other substrates) to the health-related butyrate. Butyrate concentrations were generally low after 6 h of incubation, which was expected, considering that butyrate is a secondary metabolite requiring primary production of acetate and/or lactate. Butyrate is thus typically produced during later stages of the incubation, in this case between 6 and 48 h. All products, except the trace mineral complex and the Polyphenol prebiotic blend, yielded significantly higher butyrate concentrations than the blank in at least one of their tested concentrations (FIG. 5 and Table 6). This indicates that product fermentation stimulates cross-feeding interactions that lead to butyrate production. A dose-relation was observed for these products, in which butyrate concentrations increase with product dose. By the end of the 48 h incubation, the highest butyrate concentration was obtained with the medium and high L-theanine doses. The high concentrations of the Peptide blend and the formulations with L-theanine (i.e. the combination of trace mineral complex+L-theanine+Peptides and the combination L-theanine+Peptides) also resulted in very high butyrate concentrations.

Markers for Protein Metabolism: Branched SCFA and Ammonium

Ammonium and branched SCFA (“bSCFA”, isobutyrate, isovalerate and isocaproate) production results from proteolytic microbial activity, which is associated with formation of toxic by-products such as p-cresol. Therefore, high branched SCFA and ammonium production in the colon his undesirable. As a consequence, products that reduce branched SCFA and ammonium production are considered health-beneficial.

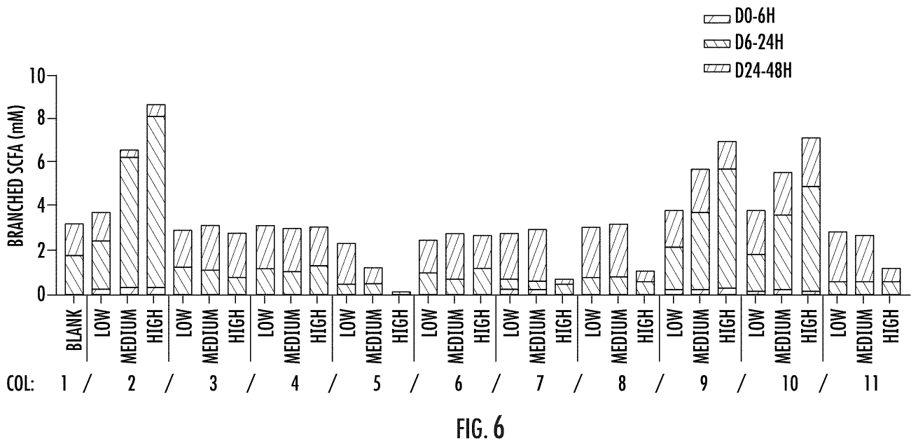

In the present experiment, production of branched SCFA mainly occurred during the 6-48 h timeframe (FIG. 6 and Table 7). The Peptide blend and two of the combination products (L-theanine+peptides, with and without trace mineral complex) stimulated the production of branched SCFA. L-theanine alone, trace mineral complex alone and Nucleotides yielded similarly low, i.e., favorable, bSCFA concentrations as the blank. XOS, the Polyphenol prebiotic blend, the full product and the positive control inulin actively lowered the production of bSCFA, with the effects being most pronounced for the high product doses.

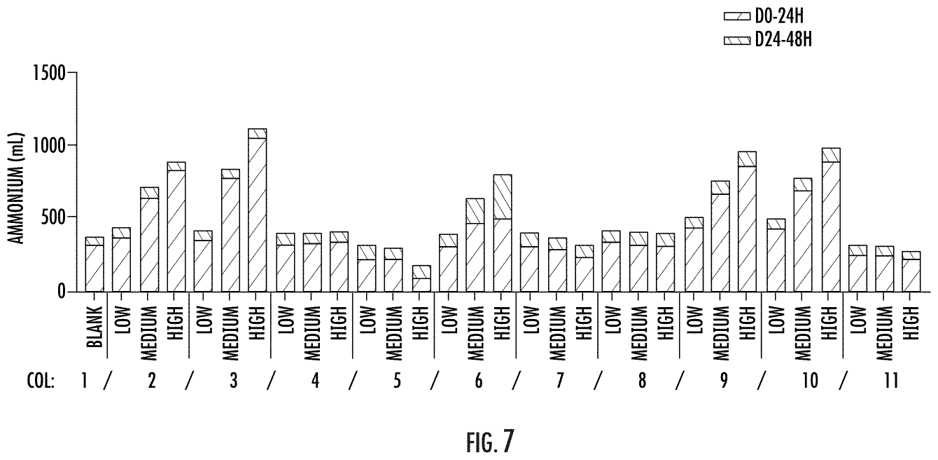

Production of ammonium occurred mainly during the first 24 h of the incubation (FIG. 7 and Table 8). The Peptide blend, both combination formulations containing L-theanine+Peptide (with and without trace mineral complex), L-theanine and the Nucleotide blend stimulated the production of ammonium. Ammonium production was related with product dose, i.e. higher doses yielded higher ammonium concentrations. The trace mineral complex and the full product yielded similar ammonium concentrations as the blank. XOS, the Polyphenol prebiotic blend and the positive control inulin actively lowered the production of ammonium, with the effects being most pronounced for the high test product doses.

TABLE 7
Branched chain production during different time intervals
upon fermentation, including blank and positive control,
inulin. (D 0-6 h, 6-24 h, 24-48 hr)

| | | Branched SCFA production | | |
|---|---|---|---|---|
| | | D 0-6 | D 6-24 | D 24-48 h |
| Blank | | 0.1 | 1.8 | 1.3 |
| Peptide blend | Low | 0.3 | 2.2 | 1.3 |
| | Medium | 0.3 | 6.0 | 0.3 |
| | High | 0.4 | 7.8 | 0.5 |
| L-theanine | Low | 0.1 | 1.2 | 1.7 |
| | Medium | 0.1 | 1.0 | 2.1 |
| | High | 0.1 | 0.7 | 2.1 |
| Trace Mineral | Low | 0.1 | 1.1 | 1.9 |
| Complex | Medium | 0.1 | 1.0 | 1.9 |
| | High | 0.1 | 1.2 | 1.8 |
| XOS prebiotic | Low | 0.1 | 0.4 | 1.9 |
| | Medium | 0.1 | 0.4 | 0.8 |
| | High | 0.0 | 0.1 | 0.0 |
| Nucleotides | Low | 0.1 | 1.0 | 1.4 |
| | Medium | 0.1 | 0.6 | 2.1 |
| | High | 0.1 | 1.1 | 1.6 |
| Polyphenol | Low | 0.2 | 0.5 | 2.2 |
| prebiotic blend | Medium | 0.2 | 0.4 | 2.5 |
| | High | 0.1 | 0.4 | 0.2 |
| Full product | Low | 0.1 | 0.7 | 2.3 |
| | Medium | 0.1 | 0.7 | 2.5 |
| | High | 0.1 | 0.5 | 0.6 |
| Trace Mineral | Low | 0.2 | 2.0 | 1.7 |
| Complex + L- | Medium | 0.2 | 3.6 | 1.9 |
| theanine + | High | 0.3 | 5.4 | 1.3 |
| peptides | | | | |
| L-theanine + | Low | 0.2 | 1.7 | 2.0 |
| peptides | Medium | 0.2 | 3.4 | 2.0 |
| | High | 0.2 | 4.9 | 2.0 |
| Inulin | Low | 0.0 | 0.6 | 2.2 |
| | Medium | 0.0 | 0.6 | 2.1 |
| | High | 0.0 | 0.6 | 0.6 |

TABLE 8
Ammonium production during different time intervals
upon fermentation, including blank and positive
control, inulin. (0-6 h, 6-24 h, 24-48 hr)

Ammonium production

| | | D 0-24 h | D 24-48 h |
|---|---|---|---|
| Blank | | 316.1 | 61.4 |
| Peptide blend | Low | 375.1 | 61.4 |
| | Medium | 626.7 | 74.0 |
| | High | 811.6 | 74.0 |
| L-theanine | Low | 344.3 | 64.4 |
| | Medium | 766.2 | 67.2 |
| | High | 1047.7 | 72.0 |
| Trace Mineral | Low | 303.7 | 77.6 |
| Complex | Medium | 326.8 | 71.4 |
| | High | 319.0 | 72.7 |
| XOS prebiotic | Low | 213.1 | 93.7 |
| | Medium | 204.4 | 82.5 |
| | High | 90.7 | 80.5 |
| Nucleotides | Low | 301.9 | 82.5 |
| | Medium | 464.4 | 176.6 |
| | High | 494.1 | 300.7 |
| Polyphenol | Low | 296.3 | 95.1 |
| prebiotic blend | Medium | 271.9 | 98.1 |
| | High | 233.3 | 68.7 |
| Full product | Low | 324.3 | 85.2 |
| | Medium | 306.9 | 85.0 |
| | High | 309.7 | 68.4 |
| Trace Mineral | Low | 422.6 | 69.6 |
| Complex + L- | Medium | 659.9 | 88.6 |
| theanine + | High | 849.0 | 93.1 |
| peptides | | | |
| L-theanine + | Low | 415.4 | 74.6 |
| peptides | Medium | 678.4 | 79.1 |
| | High | 886.0 | 82.7 |
| Inulin | Low | 242.8 | 67.3 |
| | Medium | 233.3 | 73.8 |
| | High | 216.5 | 43.4 |

Lactate Production

TABLE 9
Lactate production and consumption during different
time-intervals upon fermentation, including blank and
positive control, inulin. (0-6 h, 6-24 h, 24-48 hr)

Lactate production

| | | D 0-6 h | D 6-24 h | D 24-48 h |
|---|---|---|---|---|
| Blank | | 0.22 | −0.40 | 0.00 |
| Peptide blend | Low | 0.31 | −0.49 | 0.00 |
| | Medium | 0.82 | −0.98 | −0.02 |
| | High | 1.08 | −1.25 | −0.01 |
| L-theanine | Low | 0.21 | −0.39 | 0.00 |
| | Medium | 0.23 | −0.42 | 0.00 |
| | High | 0.23 | −0.42 | 0.00 |
| Trace Mineral | Low | 0.26 | −0.43 | −0.02 |
| Complex | Medium | 0.26 | −0.40 | 0.03 |
| | High | 0.19 | −0.36 | −0.01 |
| XOS prebiotic | Low | 4.98 | −5.16 | −0.01 |
| | Medium | 5.41 | −5.56 | −0.02 |
| | High | 11.45 | −7.54 | −4.08 |
| Nucleotides | Low | 0.21 | −0.36 | −0.04 |
| | Medium | 0.88 | −1.04 | 0.02 |
| | High | 1.22 | −1.41 | 0.02 |
| Polyphenol | Low | 0.40 | −0.56 | −0.02 |
| prebiotic blend | Medium | 1.04 | −1.22 | 0.02 |
| | High | 1.82 | −1.76 | −0.17 |
| Full product | Low | 1.59 | −1.76 | 0.01 |
| | Medium | 4.04 | −4.06 | −0.11 |
| | High | 4.90 | −4.92 | −0.11 |
| Trace Mineral | Low | 0.35 | −0.52 | −0.01 |
| Complex + L- | Medium | 0.75 | −0.90 | −0.02 |
| theanine + | High | 0.95 | −1.10 | −0.01 |
| peptides | | | | |
| L-theanine + | Low | 0.31 | −0.40 | −0.07 |
| peptides | Medium | 0.55 | −0.69 | −0.04 |
| | High | 0.98 | −1.10 | −0.04 |
| Inulin | Low | 1.62 | −1.71 | −0.10 |
| | Medium | 2.07 | −2.17 | −0.08 |
| | High | 3.57 | −3.73 | 0.00 |

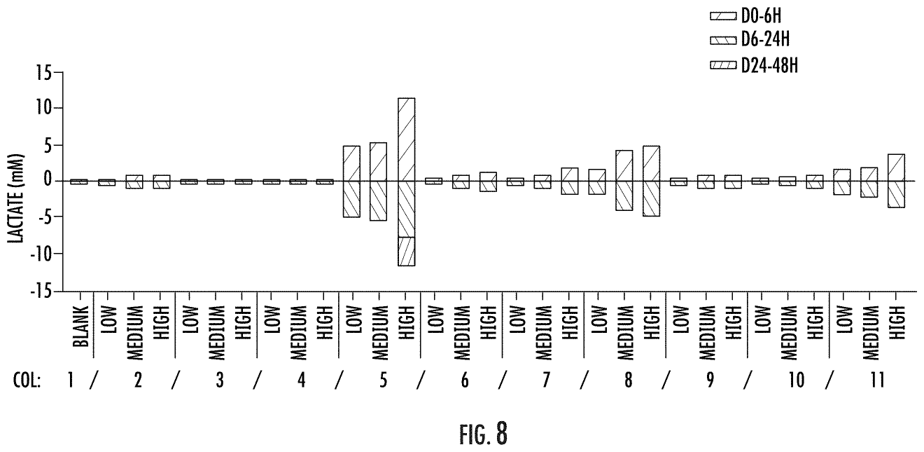

The human intestine harbors lactate-producing and lactate-utilizing (lactate converting) bacteria. Lactate is produced by lactic acid bacteria (bifidobacteria and lactobacilli); it decreases the pH of the environment. Especially at low pH values, lactate can exert strong antimicrobial effects against pathogens, as protonated lactic acid can penetrate the microbial cell, after which it dissociates and releases protons within the cell, resulting in microbial cell acidification and death. Another, indirect beneficial effect of lactate results from its conversion to the SCFAs butyrate and/or propionate, discussed above, by specific micro-organisms. As different microbial species thus produce and convert lactate, an increase of lactate concentration can result from increased lactate production, reduced lactate conversion or both. Therefore, one needs to be mindful of this when interpreting lactate data. The data, presented in FIG. 8 and Table 9 (negative values denote consumption), lead to the following observations:
As expected, the blank incubation resulted in low lactate production (and consumption) levels.
Lactate accumulation during the first 6 h (i.e. the lactate concentrations measured after 6 h of incubation) implies that the lactate production rate was higher than the lactate consumption rate in that stage of incubation. The Peptide blend, the Nucleotide blend, the Polyphenol prebiotic blend and both formulations with peptides (L-theanine+Peptides, with and without trace mineral complex) moderately stimulated lactate production during the first 6 h of the incubation; XOS, the full product and Inulin, the positive control, strongly increased lactate levels compared to the blank, with XOS achieving the highest stimulation and yielding the highest lactate concentration measured at 6 h. A dose-response was observed in the first stage of incubation in that higher product doses yielded higher lactate concentrations. L-theanine and trace mineral complex did not affect lactate production.
Overall, accumulated lactate (after 6 h) was efficiently consumed after 24 h in all reactors, which is indicative of efficient lactate conversion. However, in the incubation with the high XOS dose, a significant amount of lactate was consumed during the 24-48 h timeframe, which correlates with the high concentrations of butyrate that were obtained within the same time-interval. This indicates that at the high dose of XOS, the lactate converting bacteria needed a longer time to convert the large amount of lactate produced during the first stage of Incubation. Lactate consumption was highest for XOS, which is of course related to the higher lactate concentrations that were measured after 6 h.

Changes in Microbial Community Composition

Lactic Acid Bacteria—Lactobacillus and Bifidobacterium Spp.

TABLE 10
Abundance of Bifidobacterium in lumen and mucus
upon fermentation, including blank and positive
control, inulin. (0-6 h, 6-24 h, 24-48 hr)

| | | Bifidobacterium | | | |
|---|---|---|---|---|---|
| | | Lumen | | | Mucus |
| | | 0 h | 24 h | 48 h | 48 h |
| Blank | | 6.2E+09 | 8.8E+09 | 4.9E+09 | 1.9E+08 |
| Peptide blend | Low | 6.2E+09 | 1.0E+10 | 5.9E+09 | 1.7E+08 |
| | Medium | 6.2E+09 | 1.2E+10 | 8.9E+09 | 3.5E+08 |
| | High | 6.2E+09 | 1.4E+10 | 1.1E+10 | 1.6E+08 |
| L-theanine | Low | 6.2E+09 | 8.6E+09 | 5.7E+09 | 2.8E+08 |
| | Medium | 6.2E+09 | 1.0E+10 | 5.7E+09 | 2.0E+08 |
| | High | 6.2E+09 | 9.0E+09 | 6.6E+09 | 1.8E+08 |
| Trace Mineral | Low | 6.2E+09 | 9.7E+09 | 5.1E+09 | 9.1E+07 |
| Complex | Medium | 6.2E+09 | 1.7E+09 | 1.1E+09 | 9.1E+07 |
| | High | 6.2E+09 | 9.4E+09 | 4.9E+09 | 1.9E+08 |
| XOS prebiotic | Low | 6.2E+09 | 5.5E+10 | 4.2E+10 | 4.0E+08 |
| | Medium | 6.2E+09 | 7.9E+10 | 4.7E+10 | 6.6E+08 |
| | High | 6.2E+09 | 9.8E+10 | 5.5E+10 | 1.0E+09 |
| Nucleotides | Low | 6.2E+09 | 8.9E+09 | 5.1E+09 | 1.8E+08 |
| | Medium | 6.2E+09 | 2.2E+10 | 1.7E+10 | 3.1E+08 |
| | High | 6.2E+09 | 2.7E+10 | 2.6E+10 | 2.7E+08 |
| Polyphenol | Low | 6.2E+09 | 1.2E+10 | 6.3E+09 | 2.2E+08 |
| prebiotic blend | Medium | 6.2E+09 | 1.6E+10 | 1.0E+10 | 6.4E+08 |
| | High | 6.2E+09 | 2.2E+10 | 1.1E+10 | 6.6E+08 |
| Full product | Low | 6.2E+09 | 4.3E+09 | 2.6E+09 | 6.3E+08 |
| | Medium | 6.2E+09 | 4.1E+10 | 2.5E+10 | 6.3E+08 |
| | High | 6.2E+09 | 3.2E+10 | 2.5E+10 | 8.3E+07 |
| Trace Mineral | Low | 6.2E+09 | 1.3E+10 | 6.3E+09 | 1.8E+08 |
| Complex + L- | Medium | 6.2E+09 | 1.5E+10 | 7.6E+09 | 3.0E+07 |
| theanine + | High | 6.2E+09 | 1.7E+10 | 5.8E+09 | 7.1E+07 |
| peptides | | | | | |
| L-theanine + | Low | 6.2E+09 | 1.2E+10 | 2.4E+09 | 3.0E+07 |
| peptides | Medium | 6.2E+09 | 1.3E+10 | 4.0E+09 | 3.4E+08 |
| | High | 6.2E+09 | 1.4E+10 | 9.5E+09 | 5.6E+07 |
| Inulin | Low | 6.2E+09 | 4.9E+10 | 1.7E+10 | 4.3E+08 |
| | Medium | 6.2E+09 | 7.1E+10 | 2.6E+10 | 3.8E+08 |
| | High | 6.2E+09 | 2.7E+10 | 1.6E+10 | 3.8E+08 |

Lactobacilli and bifidobacteria are regarded as beneficial saccharolytic bacteria, capable of producing high concentrations of lactate and acetate. Lactate is an important metabolite because of its antimicrobial properties, but also (together with acetate) because it is the driver of a series of trophic interactions with other bacteria, resulting in the production of downstream metabolites.

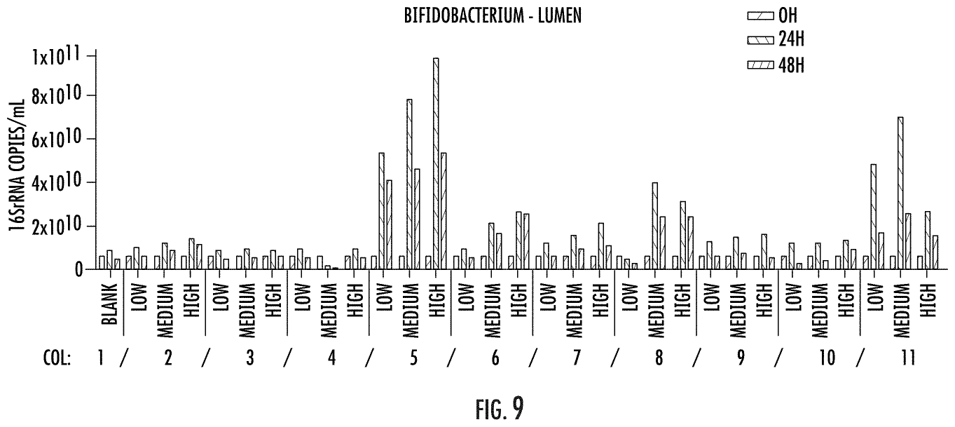

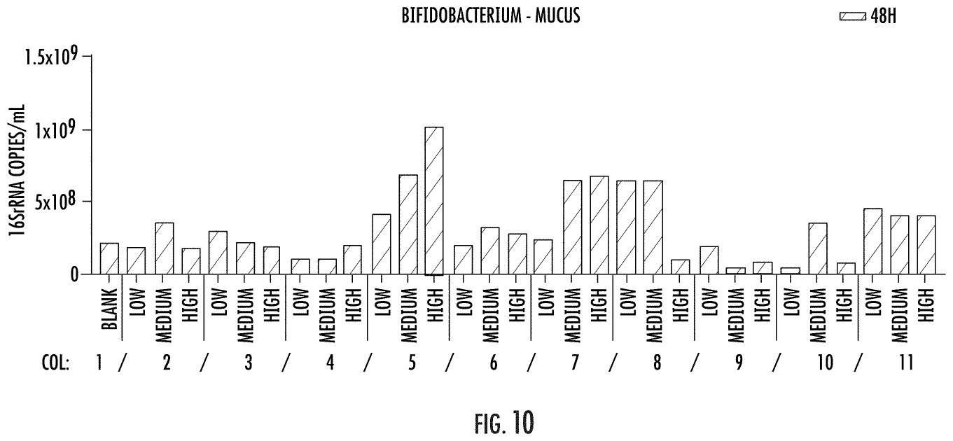

XOS and the positive control inulin strongly stimulated bifidobacteria in the lumen in a dose-related fashion (FIG. 9 and Table 10). The stimulatory effect induced by XOS was stronger than that induced by the positive control. Mild stimulatory effects were observed for the Nucleotide blend and for the full product. In the mucosal environment, an enrichment of bifidobacteria was observed in response to the treatment with XOS, and to a lesser extent in the incubations with the Polyphenol prebiotic blend and with the medium dose of the full product (FIG. 10 and Table 10).

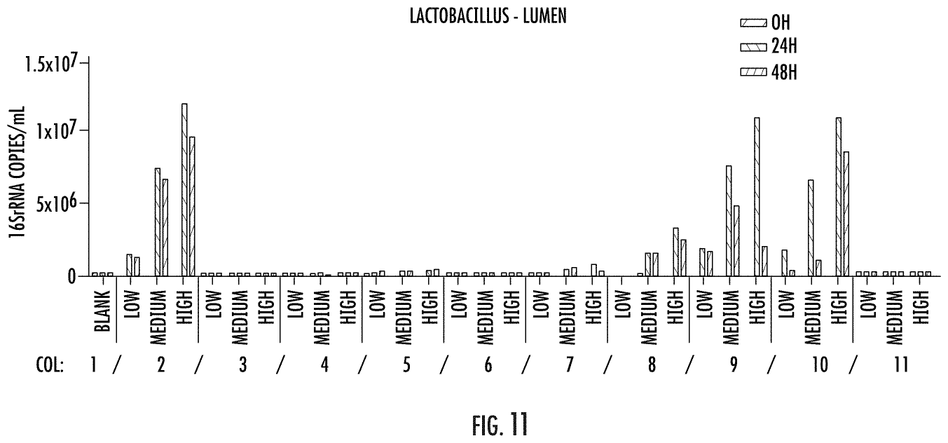

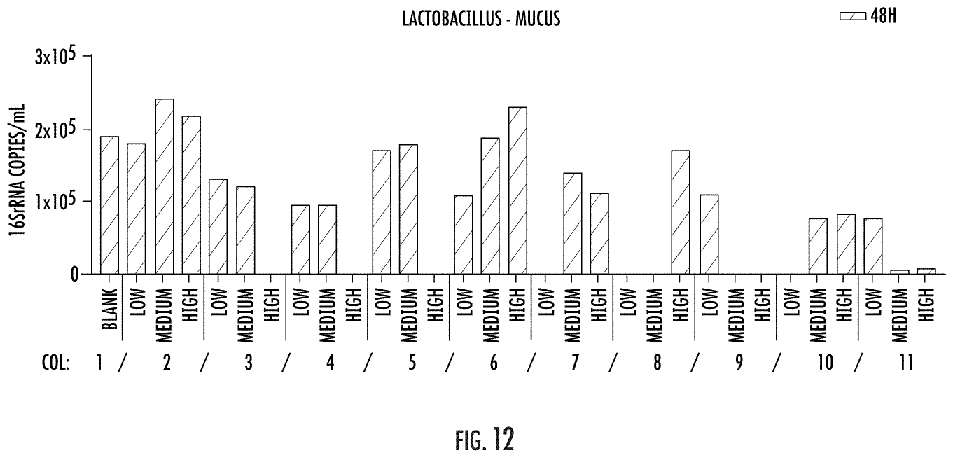

Lactobacilli were present in low abundance (<LOQ) at the start of the incubation due to low prevalence of these microbial groups in the fecal inoculum used for the experiment (FIG. 11 and Table 11). Lactobacilli were mostly simulated by the Peptide blend and by both L-theanine+Peptides formulations (with and without trace mineral complex). The full product resulted in mild stimulatory effects on lactobacilli, which can probably be attributed to its diluted content in peptides. None of the products stimulated lactobacilli in the mucosal environment (FIG. 12 and Table 11).

TABLE 11
Abundance of Lactobacillus in the lumen and mucus
upon fermentation, including blank and positive
control, inulin. (0-6 h, 6-24 h, 24-48 hr)

| | | Lactobacillus | | | |
|---|---|---|---|---|---|
| | | Lumen | | | Mucus |
| | | 0 h | 24 h | 48 h | 48 h |
| Blank | | 8.3E+04 | 7.2E+04 | 6.7E+04 | 1.9E+05 |
| Peptide blend | Low | 8.3E+04 | 1.6E+06 | 1.3E+06 | 1.8E+05 |
| | Medium | 8.3E+04 | 7.5E+06 | 6.7E+06 | 2.4E+05 |
| | High | 8.3E+04 | 1.2E+07 | 9.7E+06 | 2.2E+05 |
| L-theanine | Low | 8.3E+04 | 7.5E+04 | 8.7E+04 | 1.3E+05 |
| | Medium | 8.3E+04 | 1.2E+05 | 8.3E+04 | 1.2E+05 |
| | High | 8.3E+04 | 5.9E+04 | 8.5E+04 | 0.0E+00 |
| Trace Mineral | Low | 8.3E+04 | 8.6E+04 | 6.9E+04 | 9.4E+04 |
| Complex | Medium | 8.3E+04 | 5.0E+04 | 5.5E+03 | 9.4E+04 |
| | High | 8.3E+04 | 9.6E+04 | 9.2E+04 | 0.0E+00 |
| XOS prebiotic | Low | 8.3E+04 | 1.4E+05 | 1.6E+05 | 1.7E+05 |
| | Medium | 8.3E+04 | 1.9E+05 | 2.0E+05 | 1.8E+05 |
| | High | 8.3E+04 | 2.6E+05 | 4.1E+05 | 0.0E+00 |
| Nucleotides | Low | 8.3E+04 | 2.4E+04 | 2.7E+04 | 1.1E+05 |
| | Medium | 8.3E+04 | 7.9E+04 | 7.7E+04 | 1.9E+05 |
| | High | 8.3E+04 | 4.8E+04 | 1.6E+05 | 2.3E+05 |
| Polyphenol | Low | 8.3E+04 | 1.5E+05 | 9.8E+04 | 0.0E+00 |
| prebiotic blend | Medium | 8.3E+04 | 3.9E+05 | 4.9E+05 | 1.4E+05 |
| | High | 8.3E+04 | 7.2E+05 | 3.6E+05 | 1.1E+05 |
| Full product | Low | 8.3E+04 | 6.5E+04 | 4.6E+04 | 0.0E+00 |
| | Medium | 8.3E+04 | 1.6E+06 | 1.5E+06 | 0.0E+00 |
| | High | 8.3E+04 | 3.3E+06 | 2.5E+06 | 1.7E+05 |
| Trace Mineral | Low | 8.3E+04 | 1.8E+06 | 1.6E+06 | 1.1E+05 |
| Complex + L- | Medium | 8.3E+04 | 7.6E+06 | 4.9E+06 | 0.0E+00 |
| theanine + | High | 8.3E+04 | 1.1E+07 | 2.0E+06 | 0.0E+00 |
| peptides | | | | | |
| L-theanine + | Low | 8.3E+04 | 1.8E+06 | 2.5E+05 | 0.0E+00 |
| peptides | Medium | 8.3E+04 | 6.7E+06 | 9.8E+05 | 8.0E+04 |
| | High | 8.3E+04 | 1.1E+07 | 8.7E+06 | 8.3E+04 |
| Inulin | Low | 8.3E+04 | 1.0E+05 | 7.5E+04 | 8.0E+04 |
| | Medium | 8.3E+04 | 1.5E+05 | 6.5E+04 | 5.7E+03 |
| | High | 8.3E+04 | 5.3E+04 | 5.6E+04 | 5.7E+03 |

Propionate-Producers—Bacteroidetes and Akkermansia muciniphila

Akkermansia muciniphila is a mucin-degrading bacterium, typically prevalent in the distal colon region, and a known propionate producer. Its presence in the gut is associated with health benefits, as Inverse relationships between colonization of A. muciniphila and inflammatory conditions or obesity have been shown. A. muciniphila was present in low abundance (<LOQ (i.e. ≤1.105 copies/ml)) at the start of the incubation due to low prevalence of this microbial group in the starting inoculum used in the experiment. Moreover, none of the products stimulated A. muciniphila to as A. muciniphila remained present in abundances lower than the LOQ, i.e. lower than 1.105 copies/ml, in all incubations throughout the 48 h period.

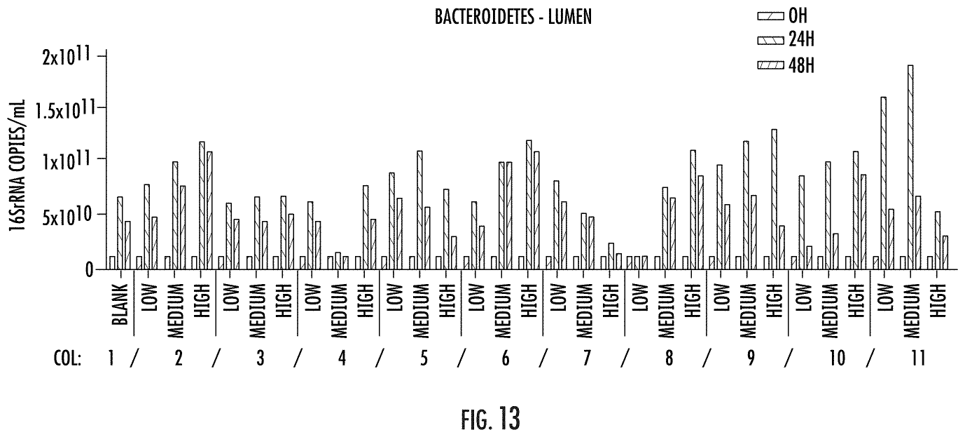

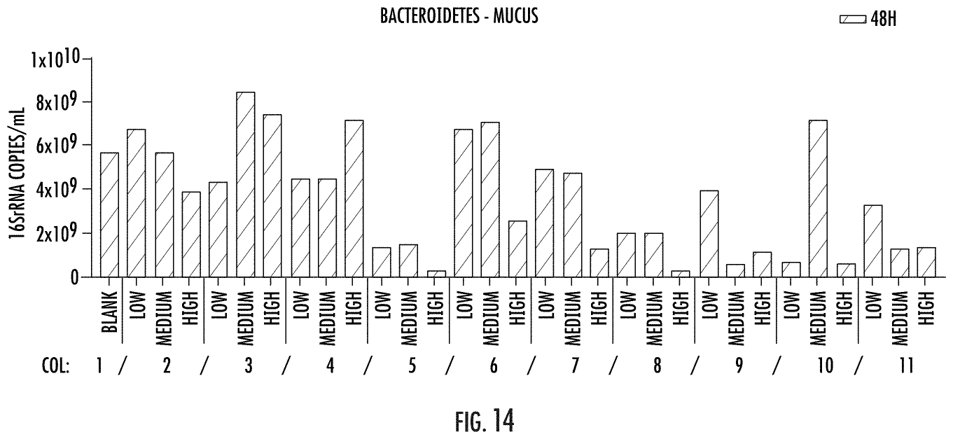

Inulin strongly stimulated Bacteroidetes in the lumen in a dose-related fashion (FIG. 13 and Table 12). Milder stimulatory effects were observed for the Peptide blend, for XOS, for the Nucleotide Wend, for the full product and for both L-theanine+Peptides formulations (with and without trace mineral complex). L-theanine and trace mineral complex did not alter the abundance of Bacteroidetes compared to the blank and the medium and high doses of the Polyphenol prebiotic blend tended to Inhibit growth of Bacteroidetes. None of the products stimulated Bacteroidetes in the mucosal environment (FIG. 14 and Table 12). However, this may have been related to the fact the Bacteroidetes population strongly colonized the mucus beads, even without treatment (i.e. 5,63.109 copies/ml in the blank), leaving less room for these bacteria to grow.

TABLE 12
Abundance of Bacteroidetes in the lumen and mucus
upon fermentation, including blank and positive
control, inulin. (0-6 h, 6-24 h, 24-48 hr)

| | | Bacteroidetes | | | |
|---|---|---|---|---|---|
| | | Lumen | | | Mucus |
| | | 0 h | 24 h | 48 h | 48 h |
| Blank | | 1.2E+10 | 6.8E+10 | 4.5E+10 | 5.6E+09 |
| Peptide blend | Low | 1.2E+10 | 7.9E+10 | 5.0E+10 | 6.7E+09 |
| | Medium | 1.2E+10 | 1.0E+11 | 7.8E+10 | 5.6E+09 |
| | High | 1.2E+10 | 1.2E+11 | 1.1E+11 | 3.8E+09 |
| L-theanine | Low | 1.2E+10 | 6.3E+10 | 4.6E+10 | 4.2E+09 |
| | Medium | 1.2E+10 | 6.8E+10 | 4.5E+10 | 8.4E+09 |
| | High | 1.2E+10 | 6.8E+10 | 5.3E+10 | 7.4E+09 |
| Trace Mineral | Low | 1.2E+10 | 6.5E+10 | 4.6E+10 | 4.4E+09 |
| Complex | Medium | 1.2E+10 | 1.6E+10 | 1.2E+10 | 4.4E+09 |
| | High | 1.2E+10 | 7.7E+10 | 4.8E+10 | 7.1E+09 |
| XOS prebiotic | Low | 1.2E+10 | 9.0E+10 | 6.6E+10 | 1.3E+09 |
| | Medium | 1.2E+10 | 1.1E+11 | 5.8E+10 | 1.4E+09 |
| | High | 1.2E+10 | 7.5E+10 | 3.0E+10 | 2.1E+08 |
| Nucleotides | Low | 1.2E+10 | 6.4E+10 | 4.1E+10 | 6.7E+09 |
| | Medium | 1.2E+10 | 1.0E+11 | 1.0E+11 | 7.0E+09 |
| | High | 1.2E+10 | 1.2E+11 | 1.1E+11 | 2.5E+09 |
| Polyphenol | Low | 1.2E+10 | 8.2E+10 | 6.3E+10 | 4.9E+09 |
| prebiotic blend | Medium | 1.2E+10 | 5.2E+10 | 4.9E+10 | 4.7E+09 |
| | High | 1.2E+10 | 2.4E+10 | 1.4E+10 | 1.1E+09 |
| Full product | Low | 1.2E+10 | 1.2E+10 | 1.2E+10 | 1.9E+09 |
| | Medium | 1.2E+10 | 7.8E+10 | 6.6E+10 | 1.9E+09 |
| | High | 1.2E+10 | 1.1E+11 | 8.7E+10 | 1.2E+08 |
| Trace Mineral | Low | 1.2E+10 | 9.7E+10 | 6.0E+10 | 3.9E+09 |
| Complex + L- | Medium | 1.2E+10 | 1.2E+11 | 6.9E+10 | 5.5E+08 |
| theanine + | High | 1.2E+10 | 1.3E+11 | 4.1E+10 | 1.1E+09 |
| peptides | | | | | |
| L-theanine + | Low | 1.2E+10 | 8.6E+10 | 2.1E+10 | 6.3E+08 |
| peptides | Medium | 1.2E+10 | 1.0E+11 | 3.3E+10 | 7.1E+09 |
| | High | 1.2E+10 | 1.1E+11 | 8.8E+10 | 5.9E+08 |
| Inulin | Low | 1.2E+10 | 1.6E+11 | 5.6E+10 | 3.2E+09 |
| | Medium | 1.2E+10 | 1.9E+11 | 6.9E+10 | 1.2E+09 |
| | High | 1.2E+10 | 5.2E+10 | 3.2E+10 | 1.2E+09 |

Butyrate Producer Faecalibacterium prausnitzii

Faecalibacterium prausnitzii has been reported as one of the main butyrate producers found in the intestine and is known for its anti-inflammatory properties, as both cell and supernatant fractions of this microorganism have anti-inflammatory activity. Low abundance of this species has been linked to disorders such as celiac disease, obesity and type 2 diabetes, appendicitis, chronic diarrhea, irritable bowel syndrome of alternating type, colorectal cancer, and particularly in inflammatory bowel disease (IBD). Many of its health-promoting characteristics have prompted those skilled in the art to consider F. prausnitzii to as a bioindicator of human health.

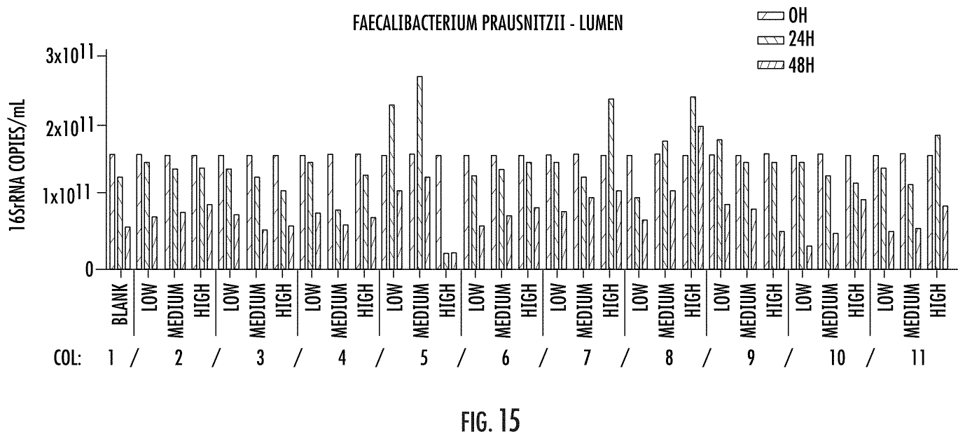

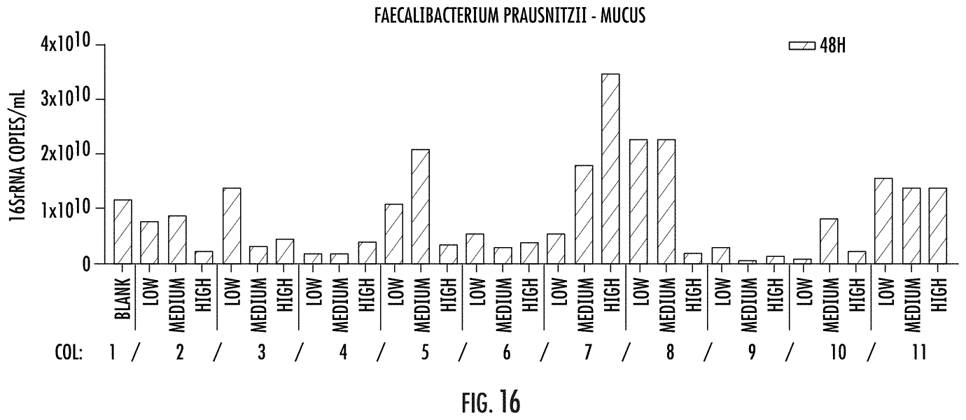

Faecalibacterium prausnitzii reached very high abundance in the lumen and in the mucosal environment in the negative control reactors (FIGS. 15 and 16, respectively, and Table 13). Enrichments in such dense populations are often less visible, yet biologically meaningful as they imply a strong enrichment of the microbial community. For instance, a log increase in a population consisting of 1×10⁹ copies/ml implies an enrichment with 9×10⁹ copies/ml, whereas a log increase in a population consisting of 1×10⁶ copies/ml implies an enrichment with 9×10⁹ copies/ml. Hence, from that perspective, XOS, the Polyphenol prebiotic blend and the full product were found to stimulate the already dense community of F. prausnitzii in the lumen and mucosal environment.

TABLE 13
Abundance of Faecalibacterium prausnitzii in the lumen
and mucus upon fermentation, including blank and positive
control, inulin. (0-6 h, 6-24 h, 24-48 hr)

| | | Faecalibacterium prausnitzii | | | |
|---|---|---|---|---|---|
| | | Lumen | | | Mucus |
| | | 0 h | 24 h | 48 h | 48 h |
| Blank | | 1.6E+11 | 1.3E+11 | 6.1E+10 | 1.2E+10 |
| Peptide blend | Low | 1.6E+11 | 1.5E+11 | 7.3E+10 | 7.8E+09 |
| | Medium | 1.6E+11 | 1.4E+11 | 8.0E+10 | 9.0E+09 |
| | High | 1.6E+11 | 1.4E+11 | 9.1E+10 | 2.2E+09 |
| L-theanine | Low | 1.6E+11 | 1.4E+11 | 7.6E+10 | 1.4E+10 |
| | Medium | 1.6E+11 | 1.3E+11 | 5.7E+10 | 3.5E+09 |
| | High | 1.6E+11 | 1.1E+11 | 6.3E+10 | 4.5E+09 |
| Trace Mineral | Low | 1.6E+11 | 1.5E+11 | 7.6E+10 | 1.8E+09 |
| Complex | Medium | 1.6E+11 | 8.5E+10 | 6.3E+10 | 1.8E+09 |
| | High | 1.6E+11 | 1.3E+11 | 7.4E+10 | 4.2E+09 |
| XOS prebiotic | Low | 1.6E+11 | 2.3E+11 | 1.1E+11 | 1.1E+10 |
| | Medium | 1.6E+11 | 2.7E+11 | 1.3E+11 | 2.1E+10 |
| | High | 1.6E+11 | 2.2E+10 | 2.4E+10 | 3.6E+09 |
| Nucleotides | Low | 1.6E+11 | 1.3E+11 | 6.1E+10 | 5.5E+09 |
| | Medium | 1.6E+11 | 1.4E+11 | 7.4E+10 | 3.1E+09 |
| | High | 1.6E+11 | 1.5E+11 | 8.5E+10 | 3.8E+09 |
| Polyphenol | Low | 1.6E+11 | 1.5E+11 | 7.9E+10 | 5.4E+09 |
| prebiotic blend | Medium | 1.6E+11 | 1.3E+11 | 1.0E+11 | 1.8E+10 |
| | High | 1.6E+11 | 2.4E+11 | 1.1E+11 | 3.5E+10 |
| Full product | Low | 1.6E+11 | 1.0E+11 | 7.0E+10 | 2.3E+10 |
| | Medium | 1.6E+11 | 1.8E+11 | 1.1E+11 | 2.3E+10 |
| | High | 1.6E+11 | 2.4E+11 | 2.0E+11 | 1.8E+09 |
| Trace Mineral | Low | 1.6E+11 | 1.8E+11 | 8.9E+10 | 3.0E+09 |
| Complex + L- | Medium | 1.6E+11 | 1.5E+11 | 8.4E+10 | 6.7E+08 |
| theanine + | High | 1.6E+11 | 1.5E+11 | 5.4E+10 | 1.3E+09 |
| peptides | | | | | |
| L-theanine + | Low | 1.6E+11 | 1.5E+11 | 3.2E+10 | 9.6E+08 |
| peptides | Medium | 1.6E+11 | 1.3E+11 | 4.9E+10 | 8.2E+09 |
| | High | 1.6E+11 | 1.2E+11 | 9.7E+10 | 2.1E+09 |
| Inulin | Low | 1.6E+11 | 1.4E+11 | 5.2E+10 | 1.6E+10 |
| | Medium | 1.6E+11 | 1.2E+11 | 5.7E+10 | 1.4E+10 |
| | High | 1.6E+11 | 1.9E+11 | 8.9E+10 | 1.4E+10 |

TABLE 14
Abundance of Firmicutes in lumen and mucus upon
fermentation, including blank and positive
control, inulin. (0-6 h, 6-24 h, 24-48 hr)

| | | Firmicutes | | | |
|---|---|---|---|---|---|
| | | Lumen | | | Mucus |
| | | 0 h | 24 h | 48 h | 48 h |
| Blank | | 1.0E+10 | 3.3E+10 | 1.5E+10 | 2.6E+10 |
| Peptide blend | Low | 1.0E+10 | 3.8E+10 | 1.7E+10 | 2.4E+10 |
| | Medium | 1.0E+10 | 5.1E+10 | 3.0E+10 | 3.9E+10 |
| | High | 1.0E+10 | 5.2E+10 | 3.7E+10 | 3.6E+10 |
| L-theanine | Low | 1.0E+10 | 3.5E+10 | 1.8E+10 | 2.4E+10 |
| | Medium | 1.0E+10 | 4.5E+10 | 1.9E+10 | 3.5E+10 |
| | High | 1.0E+10 | 5.6E+10 | 3.0E+10 | 4.3E+10 |
| Trace Mineral | Low | 1.0E+10 | 3.2E+10 | 1.7E+10 | 3.3E+10 |
| Complex | Medium | 1.0E+10 | 1.1E+10 | 7.1E+09 | 3.3E+10 |
| | High | 1.0E+10 | 3.3E+10 | 1.8E+10 | 2.6E+10 |
| XOS prebiotic | Low | 1.0E+10 | 6.2E+10 | 2.7E+10 | 1.2E+10 |
| | Medium | 1.0E+10 | 7.4E+10 | 3.5E+10 | 2.4E+10 |
| | High | 1.0E+10 | 9.3E+10 | 4.2E+10 | 1.6E+09 |
| Nucleotides | Low | 1.0E+10 | 3.4E+10 | 1.7E+10 | 2.5E+10 |
| | Medium | 1.0E+10 | 7.2E+10 | 5.7E+10 | 2.9E+10 |
| | High | 1.0E+10 | 1.0E+11 | 7.3E+10 | 1.3E+10 |
| Polyphenol | Low | 1.0E+10 | 3.9E+10 | 2.2E+10 | 1.5E+10 |
| prebiotic blend | Medium | 1.0E+10 | 3.9E+10 | 3.1E+10 | 2.5E+10 |
| | High | 1.0E+10 | 3.3E+10 | 1.7E+10 | 2.9E+10 |
| Full product | Low | 1.0E+10 | 1.0E+10 | 7.9E+09 | 3.8E+10 |
| | Medium | 1.0E+10 | 6.4E+10 | 4.0E+10 | 3.8E+10 |
| | High | 1.0E+10 | 1.1E+11 | 6.6E+10 | 1.3E+09 |
| Trace Mineral | Low | 1.0E+10 | 5.4E+10 | 3.0E+10 | 2.4E+10 |
| Complex + L- | Medium | 1.0E+10 | 6.2E+10 | 2.9E+10 | 2.9E+09 |
| theanine + | High | 1.0E+10 | 7.1E+10 | 2.2E+10 | 5.9E+09 |
| peptides | | | | | |
| L-theanine + | Low | 1.0E+10 | 4.9E+10 | 8.9E+09 | 4.6E+09 |
| peptides | Medium | 1.0E+10 | 6.4E+10 | 1.6E+10 | 2.5E+10 |
| | High | 1.0E+10 | 7.5E+10 | 3.8E+10 | 4.5E+09 |
| Inulin | Low | 1.0E+10 | 8.9E+10 | 2.0E+10 | 1.7E+10 |
| | Medium | 1.0E+10 | 1.2E+11 | 2.3E+10 | 1.6E+10 |
| | High | 1.0E+10 | 3.6E+10 | 1.6E+10 | 1.6E+10 |

The Firmicutes phylum is a very diverse bacterial phylum, containing acetate—(for instance 14 Abundance and 14 abundance), lactate—(for instance Lactobacillae and Eubacteriaceae), propionate—(for instance Veillonellaceae and Acidaminococcaceae) and butyrate-producers (for Instance Lachnospiraceae and Ruminococcaceae).

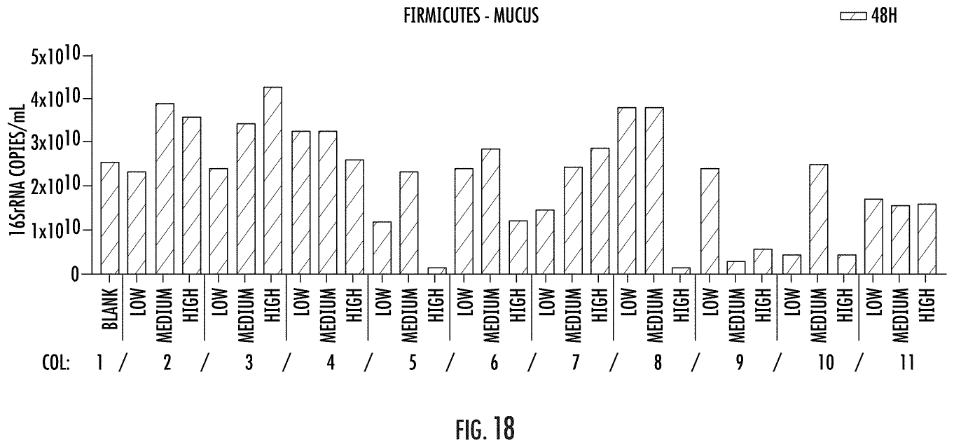

The test products XOS, nucleotide blend, full product and positive control inulin each strongly stimulated Firmicutes in the lumen in a dose-related fashion (FIG. 18 and Table 14). Mild stimulatory effects were observed for the L-theanine+Peptides formulations (with or without trace mineral complex). Regardless of the strong colonization of the mucus beads in the blank incubation (i.e. 2,56.1010 copies/ml), enrichment of Firmicutes was recorded upon treatment with the Peptide blend, L-theanine and the full product.

From the results of the foregoing experiments in this Example 1, the following conclusions can be reached:

First, the full product was efficiently fermented (i.e., lowered pH and/or increased gas production and/or increased one or more SCFA) by the gut microbiota of the adult donor under investigation, resulting in elevated acetate, propionate and butyrate levels that proportionally increased as the dose of the product was elevated. Likewise, the full product stimulated lactate production in a dose-related fashion.

Simulation of acetate and lactate production indicates a correlation with observed enrichment of two groups of health-related lactic acid bacteria (bifidobacteria and lactobacilli), while effects on propionate production were related to stimulation of members of the Bacteroidetes and Firmicutes phyla (likely Veillonellaceae family). Further, effects on butyrate production could be linked to stimulatory effects on the Firmicutes phylum that contains multiple butyrate producers such as F. prausnitzii that was indeed found to increase (for the two doses tested).

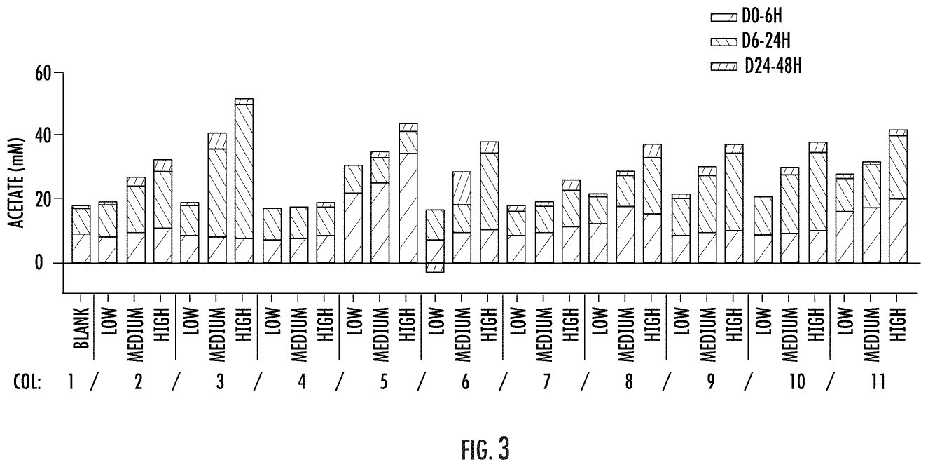

Besides the full product, different individual ingredients were tested which ingredients or combinations of ingredients short of the full product were active in the various premises tested. Many Ingredients and the combinations tested were fermented by the gut microbiota, resulting in elevated SCFA (acetate, propionate and butyrate) and lactate levels. Particularly notable, indeed surprising, was the ability of theanine to contribute to SCFA formation (see, e.g., FIG. 2 and Table 6; FIG. 3, Table 5 and FIG. 5. Table 6). Fermentation started already during the first 6 h of incubation, yielding metabolites that decrease the pH of the environment. XOS, the full product and the positive control inulin stimulated acetate, lactate and propionate production during the first 6 h of the incubation. Production of acetate and lactate suggests the action of lactic acid bacteria, while production of propionate suggests that also Bacteroidetes spp., typically capable of degrading complex molecules, may have been involved in substrate breakdown. qPCR data confirmed this hypothesis, in the sense that each of these treatments were consistently associated with an enrichment of bifidobacteria, thereby leading to the conclusion that XOS the full product and the positive control increased the efficiency of fermentation by increasing the population of bacteria. On the contrary, because lactobacilli (also lactic acid bacteria) were detected in low abundances in the fecal material of the investigated donor, they were not, or were only moderately stimulated by the applied treatments, as growth is influenced by the initial bacterial population The high abundances of members of the Bacteroidetes phylum in the fecal material of the donor and their enrichment by XOS, the full product and the positive control inulin are consistent with and likely to explain the stimulatory effects that were observed on propionate production during early stages of the incubations (0 to 6 h).

All products, except L-theanine, the trace mineral complex and the Polyphenol prebiotic blend stimulated propionate production, suggesting the action of members of the Bacteroidetes phylum and/or Veillonellaceae family (belonging to Firmicutes). These bacterial groups are both capable of propionate production, the former through direct degradation of (complex) carbohydrates, the latter through conversion of lactate. These data correlate well with qPCR data on Bacteroidetes members, which were specifically enriched by the aforementioned treatments. These results thus illustrate that the Peptide component, XOS, the Nucleotide Wend, the full product, the L-theanine+peptides combination and inulin enriched Bacteroidetes members, which resulted in increased propionate concentrations.

All products, except the trace mineral complex and the Polyphenol prebiotic, stimulated butyrate production. Butyrate production results from cross-feeding interactions between members of the gut microbial community, i.e. conversion of lactate by species such as Anaerobutyricum hallil and Anaerostipes caccae and from conversion of acetate by Roseburia spp., Faecailbacterium prausnitzii and Eubacterium rectale. qPCR data illustrated that XOS and the full product stimulated F. prausnitzii in the mucosal and luminal environment, which is likely to have contributed to the increased butyrate levels in these incubations.

Finally, XOS, the Polyphenol prebiotic and the positive control inulin significantly lowered production of branched SCFA and ammonium.

In summary, the full product was efficiently fermented by the gut microbiota of the adult donor under investigation, resulting in elevated acetate, propionate and butyrate levels that proportionally increased as the dose of the product was elevated. Likewise, the full product stimulated lactate production in a dose-related fashion. Simulation of acetate and lactate production was likely correlated with observed enrichment of two groups of health-related lactic acid bacteria (bifidobacteria and lactobacilli), while effects on propionate production were related to stimulation of members of the Bacteroidetes and Firmicutes phyla (likely Vellonellaceae family). Further, effects on butyrate production could be linked to stimulatory effects on Firmicutes that contains multiple butyrate producers such as F. prausnitzii that was indeed found to increase (for the two doses tested). Besides the full product, different individual ingredients were tested to assess whether they contributed to the observed effects of the complete product. Interestingly, many ingredients were fermented by the gut microbiota, resulting in elevated SCFA (acetate, propionate and butyrate) and lactate levels. XOS and the positive control inulin stimulated acetate, lactate and propionate production during the first 6 h of the incubation, attributed to the stimulation of bifidobacteria and members of the Bacteroidetes phylum. Further, the Peptide blend, XOS, the Nucleotide blend, the L-theanine+Peptides formulations and inulin enriched Bacteroidetes members during the 48 h incubation period, which was translated into increased propionate concentrations. A notable finding was that the initial fermentation of XOS was very strong and more profound than the positive control Inulin. Next, all products, except the trace mineral complex used alone (it is not fermentable) and the Polyphenol prebiotic blend, stimulated butyrate production, with a very strong and indeed surprising stimulation of butyrate for L-theanine. XOS activity can be correlated to the enrichment of F. prausnitzii in the mucosal and luminal environments, a known butyrate-producer. Finally, XOS, the Polyphenol prebiotic blend and the positive control inulin significantly lowered production of branched SCFA and ammonium.

### Example 2: In Vitro Assessment of Mucin Production and Tight Junction Integrity

The micro-organisms in the gut represent a biologically active community which lies at the interface of the host with its nutritional environment. As a consequence, they profoundly influence several aspects of the physiology and metabolism of the host. A wide range of microbial structural components and metabolites directly interact with host intestinal cells to influence nutrient uptake and epithelial health. Both microbial associated molecular patterns (MAMPs) and bacterial-derived metabolites (e.g. short-chain fatty acids (SCFA)) activate various signaling pathways such as lymphocyte maturation, epithelial health, neuroendocrine signaling, pattern recognition receptors (PRRs)-mediated and G-protein coupled receptor (GPRs)-mediated signaling. In turn, these signaling pathways will dictate inflammatory tone, energy balance, gut motility and appetite regulation (reviewed in Ha, C. W. Y., et al. (2014) W.J. Gastroenterol., 20(44):16498-16517). Dysregulation of host-microbiome Interactions has been recognized to contribute to numerous diseases, including metabolic syndrome and obesity, inflammatory bowel diseases (IBD) such as Crohn's disease (CD) and ulcerative colitis (UC), irritable bowel syndrome (IBS), celiac disease, diabetes, allergies, asthma and autoimmune diseases (Groschwitz, K. R. and Hogan, S. P. (2009) J. Allergy Clin. Immunol., 124(1):3-20). Common to these disorders is the dysregulation of the intestinal epithelial barrier (more permeable), initiating the pathology (Fasano, A. (2011) Physiol. Rev., 91: 151-175). When the intestinal barrier function is disrupted, the trafficking of molecules is no longer under control, so that luminal contents may enter the lamina propria and activate the immune system, thereby leading to uncontrolled immune responses (a process known as ‘leaky gut’). The intestinal epithelial barrier is formed by intercellular tight junctions, a complex protein-protein network that mechanically links adjacent cells and seals the intercellular space. Therefore, the intestinal epithelial barrier controls the equilibrium between immune tolerance and immune activation and so it has a prominent role in ‘leaky gut’ pathogenesis. An improper functioning or regulation of these tight junctions seems to be responsible for larger intercellular spaces allowing luminal element passage through the barrier, with a consecutive local and systemic inflammation. Accordingly, devising substances and methods to restore such equilibrium (or to ameliorate disequilibrium) by improving functioning of tight junctions is highly desirable.

In order to obtain additional information on modulatory effects of the present compositions and methods on gut surface, an in vitro model representative of a healthy epithelial cell layer was utilized. The Caco-2 cell line is a human intestinal epithelial-like cell line, widely used as an in vitro model of the small intestinal mucosa to predict the absorption of orally administered drugs. Caco-2 cells can be grown into a confluent monolayer that provides a physical and biochemical barrier to the passage of ions and small molecules. At this stage, cells are differentiated and polarized in such way that, both morphologically and functionally, they resemble the enterocyte lining in the human small intestine.

A co-culture model known in the art (Loyoza-Aguillo, I. et al, Mol Pharm. 2017 Apr. 3; 14(4):1264-1270. doi: 10.1021/acs.molpharmaceut.6b01165. Epub 2017 Mar. 10) which comprises enterocytes (Caco-2) and HT-29-mtx cells which display goblet cell-like characteristics (Lesuffleur, T., et al. (1990) Cancer research, 50: 6334.6343) was utilized for evaluation of tight junctions and mucin production in response to the treatments with the test products used for the experiments of Example 1 or the combinations of components noted in Example 1 including the full product. Caco-2, when seeded on suitable supports, spontaneously differentiate Into mature enterocyte-like cells, characterized by polarization, presence of villi, formation of domes, presence of tight junctions and vectorial transport and expression of apical brush-border enzymes (reviewed by Sambuy, Y., et al. (2005) Cell Biology and Toxicology. 21: 1-26). The mucus layer is mimicked by co-culturing Caco-2 cells with HT29-MTX cells, a mucin producing human cell line. This model is considered a useful in vitro intestinal model.

Barrier function was evaluated by measuring TEER and expression of tight junction and mucin genes in the co-culture model using TNF alpha/IFN gamma as an inflammatory insult. Inulin and sodium butyrate were used as positive controls. The test products were administered at three different concentrations.

The test product amounts were determined by titration, as described in Example 1, but using the co-culture to determine the highest nontoxic amount.

Tight junction proteins include occludin, junctional adhesion molecules (JAM) and claudins¹. Occludin and JAM have a regulatory role, while claudins are transmembrane proteins are mainly responsible for the intestinal barrier function. Occludin and claudins link adjacent cells to the actin cytoskeleton through cytoplasmic scaffolding proteins like zonula occludens (ZO) proteins. Alterations in intestinal permeability related to changes in tight junction competency are linked to IBD and IBS (Fasano, A. et al, supra; Bischoff, S. C. et al, (2014) BMC Gastroenterology, 14: 189).

Claudin proteins form the structural backbone of tight junctions. In the intestine, claudin-1, -3, -4, -5 and -8 strengthen tight junctions, with decreased paracellular permeability. On the other hand, claudin-2 forms charge-selective paracellular pores and is selectively permeable to small cations and water. Claudin-2 can be induced by pathogens as a means to clear the infection. In addition, claudin-2 is suggested to contribute to ‘leaky gut’ under pathological conditions. In HT29 cells, claudin-2 expression was downregulated by butyrate; while claudin-1, occludin and zonula occludens (ZO-1 and ZO-2) were upregulated. In addition, inflammatory stimuli, like TNFα and IFNγ, increased expression of claudin-2; while they decreased the expression of occludin (Luettig, J. et al. (2015) Tissue barriers 3(1-2); Ye, D. et al. (2011) Gastroenterology 141: 1323-1333; and Mankertz, J. et al. (2000) Journal of Cell Science 113: 2085-2090). In addition, probiotic strains were shown to increase the expression of occludin (Bischoff, S. C. et al, supra; Luettig, J. et al, supra). In CD patients, reduced expression of occludin and claudins-3, -5 and -8 was reported, in addition to upregulation of claudin-2 (Bischoff, Luettig, both supra). In UC patients, occludin and claudins-1, -4 and -7 were downregulated; while claudin-3 expression was unchanged. Also, claudin-2 was upregulated in UC patients.

The mucus layer contains various mucosal secretions, like mucins, trefoil peptides and surfactant lipids. Two types of mucins exist, namely secreted and membrane-bound mucins². MUC2 is the main mucin secreted in the Intestine, but most of the membrane-bound mucins are also expressed in the small and large intestine. Membrane-bound mucins are involved in cell signaling, immune modulation, growth, adhesion and motility; while secreted mucins form a protective layer, which creates a physical barrier against pathogens. Disruption of the mucus layer exposes the mucosa to the microbiome and may play a role in the pathogenesis of IBD. In addition, mucus contains trefoil factors, defensins and secreted immunoglobulins, promoting wound healing and mucosal restoration at damaged sites in the epithelium. Finally, mucus also contains antimicrobial agents, providing protection to the mucosal environment from invading pathogens.

In healthy ileal mucosa from CD patients, MUC2 and MUC3 are mainly expressed.

In the present experiment, the colonic suspensions collected from the SHIME in Example 1 were brought in contact with the apical side of the co-cultures. This approach allows evaluating the effect Induced by not only the test product but also the fermentation-derived metabolites produced by the gut microbiota during the digestive steps.

Cell Cultures. Caco-2 cells (HTB-37) were obtained from the American Type Culture Collection (ATCC, Manassas, Va.). Cells at passage 18 were seeded at a density of 10.000 cells/cm² and grown with 7-day passage frequency in Dulbecco's modified Eagle medium (DMEM) containing 25 mM glucose and 4 mM glutamine and supplemented with 0.1% (v/v) nonessential amino acids (Invitrogen) and 10% (v/v) heat-inactivated fetal bovine serum (FBS). Cells are incubated at 37° C. in a humidified atmosphere of air/CO2 95:5, v/v). The Caco-2 cells were seeded in a ratio 90:10 with HT-29-mtx cells. The experiment is conducted in a 24-well plate with semipermeable inserts, previously coated with collagen for adhesion.

The co-cultured cells are differentiated for 14 days with 3 medium changes per week.

Caco-2/HT29-MTX monolayers were cultured for 14 days, with three medium changes/week, until a functional cell monolayer with a transepithelial electrical resistance (TEER) was obtained. Cells were maintained in Dulbecco's Modified Eagle Medium (DMEM) containing glucose and glutamine and supplemented with HEPES and 20% (v/v) heat-inactivated (HI) fetal bovine serum (FBS). Before treatment, the TEER of the Caco-2/HT29-MTX monolayers was measured (0 h time point). The TEER of an empty insert was subtracted from all readings to account for the residual electrical resistance of an insert. The apical compartment (containing the Caco-2/HT29-MTX cells) was filled with sterile-filtered (0.22 μm) colonic batch suspensions. Cells were also treated apically with Sodium butyrate (NaB) (Sigma-Aldrich) as positive control. In addition, cells were stimulated from the basolateral side with TNFα/IFNγ. Cells were also exposed to Caco-2 complete medium in both chambers as control. Cells were treated for 48 h and the TEER was measured at 24 h (=24 h time point) and 48 h (=48 h time point). After subtracting the TEER of the empty insert, all 24 h and 48 h values were normalized to its own 0 h value (to account for the differences in initial TEER of the different inserts) and are presented as percentage of initial value. Then, cells were lysed in RNA lysis buffer and samples were kept at −80° C. All treatments with colonic batch suspensions were done as singlets. Cells were incubated at 37° C. in a humidified atmosphere of air/CO2 (95:5, v/v).

RNA isolation and qPCR. RNA from cells was isolated using the ISOLATE II RNA Micro kit from GC Biotech; according to manufacturer's instructions. cDNA was prepared using the high-capacity cDNA reverse transcription kit (Applied Biosystems) in a Thermal Cycler Biometra (Westburg). qPCRs were performed on a Quantstudio 5 (Applied Biosystems) with the Sensifast SYBR LO-ROX mix (Bioline).

On the day of the experiment, TEER (transepithelial electrical resistance) is measured (corresponding to a time point of 0 h) to assess the integrity of the monolayer.

Cells are apically incubated for 48 h with the colon batch samples collected from Example 1 diluted in cell culture medium. Sodium butyrate (NaB) was used as positive control; cell culture medium will be used as negative control. At the same time, cells were basolaterally stimulated with TNFalpha/IFNgamma to induce damage (inflammatory insult) for 48 h. A TNFalpha/IFNgamma only control was included.

TEER was measured again at 24 h and 48 h of incubation.

After 48 h, cells were lysed in RNA lysis buffer, centrifuged and the DNA collected. qPCR is performed for tight junction genes (claudin-1, -2, -3 and -4, and occludin) and mucin genes (mucin 3); 2 housekeeping genes were Included. The purpose of this is to evaluate improvements in tight junction and mucin production, each as reflected in increased expression of the TJ and mucin genes. Mucins can be measured as disclosed for example in Leteutre, E. et al, Biology of the Cell 96 (2004) 145-151. Mucin turnover can be assessed as described for example in Schneider, H. et al, Nature Scientific Reports | (2018) 8:5760 | DOI:10.1038/s41598-018-24148-x.

Transepithelial electrical resistance (TEER). Batch samples were collected 48 h after addition of the different treatments. These samples were diluted (1:5, v/v) in complete medium (CM) after filter-sterilization (0.22 μm) and were given apically to the Caco-2/HT29-MTX co-cultures for 48 h.

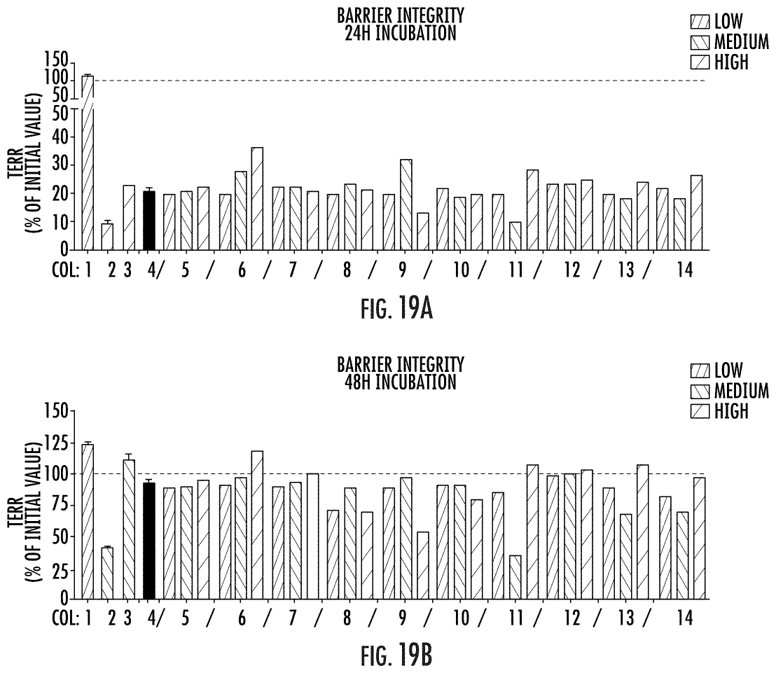

Simultaneously, cells were stimulated from the basolateral side with TNFα/IFNγ to simulate inflammation-induced barrier disruption. The results are shown in FIG. 19A for the 24-hour incubation and 198 for the 48-hour incubation. CM means complete medium control.

TNFα/IFNγ treatment induced a 90% decrease in TEER upon 24 h of Incubation; which spontaneously increased again to 40% of its initial value after 48 h (FIG. 19). Upon 24 h of incubation, TEER did decrease by approximately 80% in the presence of the positive control Sodium butyrate (NaB). However, as expected, NaB increased the TEER above its initial value upon 48 h of incubation; demonstrating its protective effects on inflammation-induced barrier disruption.

Treatment with the colonic control suspensions (i.e. blank) also decreased the TEER by 80% upon 24 h of incubation. In contrast, after 48 h of incubation, TEER increased again to approximately 90% of its initial value. Therefore, colonic suspensions as such were already able to protect the intestinal epithelial barrier from inflammation-induced damage; which was expected.

After 24 h of incubation, for most treatments and concentrations, TEER decreased similarly to the blank control. However, remarkably, treatment with L-theanine increased the TEER in a concentration-dependent manner compared to the blank. Also, L-theanine+peptides slightly increased the TEER at the highest concentration; TEER was also slightly higher at all concentrations of trace mineral complex+L-theanine+peptides; compared to the blank control, even though the combinations have proportionally less L-theanine than the L-theanine test product when equal weights of material are compared. The full product increased the TEER at the highest concentration, protecting the Intestinal barrier from inflammation-induced damage; however; at the medium concentration a decrease was noted. Nucleotides increased the TEER at the medium concentration; while it decreased TEER at its highest concentration. The inulin control showed a slight increase in TEER at the highest concentration compared to the blank control.

After 48 h of incubation, L-theanine increased the TEER in a concentration-dependent manner compared to the blank control. Furthermore, the highest concentration of L-theanine strongly Increased the TEER above its initial value. In addition, the highest concentration of the trace mineral complex increased the TEER compared to the blank control to its initial levels. This result persisted in a model where an inflammation insult was inflicted on the cultured cells. Accordingly, these results suggest that the minimum effective amount of trace mineral complex should be increased.

The results also suggest that a combination of L-theanine and trace mineral complex would be beneficial.

The lowest and medium concentrations of trace mineral complex+L-theanine+peptides increased the TEER to its initial levels; while at the highest concentration, the TEER even further increased. The combination of L-theanine+peptides increased the TEER above its initial levels at the highest concentration; as was the case for the full product. Note that the medium concentration of the full product strongly decreased the TEER compared to the blank. As this was only 1 measurement, a technical error in dosing cannot be excluded. At the highest concentration, the inulin control increased the TEER almost to its initial levels. Finally, the highest concentration of nucleotides decreased the TEER in this experiment but there is other experimental evidence showing the contrary: an increase in TEER.

From the foregoing, he strongest protection against inflammation-induced barrier disruption was seen for L-theanine. Furthermore, the trace mineral complex, combination containing peptides and L-theanine (with and without the trace mineral complex) increased TEER. The full product also showed substantially increased protection, even though it contained a fraction of the L-theanine and a fraction of the peptides compared to these components used individually (see Example 1). The protective effects were most apparent at the highest concentrations used.

The second part of this experiment involved assessment of tight junction and mucin genes.

After 48 h of incubation, Caco-2/HT29-MTX cells were lysed and qPCR was performed to determine the expression of tight junction (claudin-1, -2, -3, -4 and occludin) and mucin (MUC3) genes.

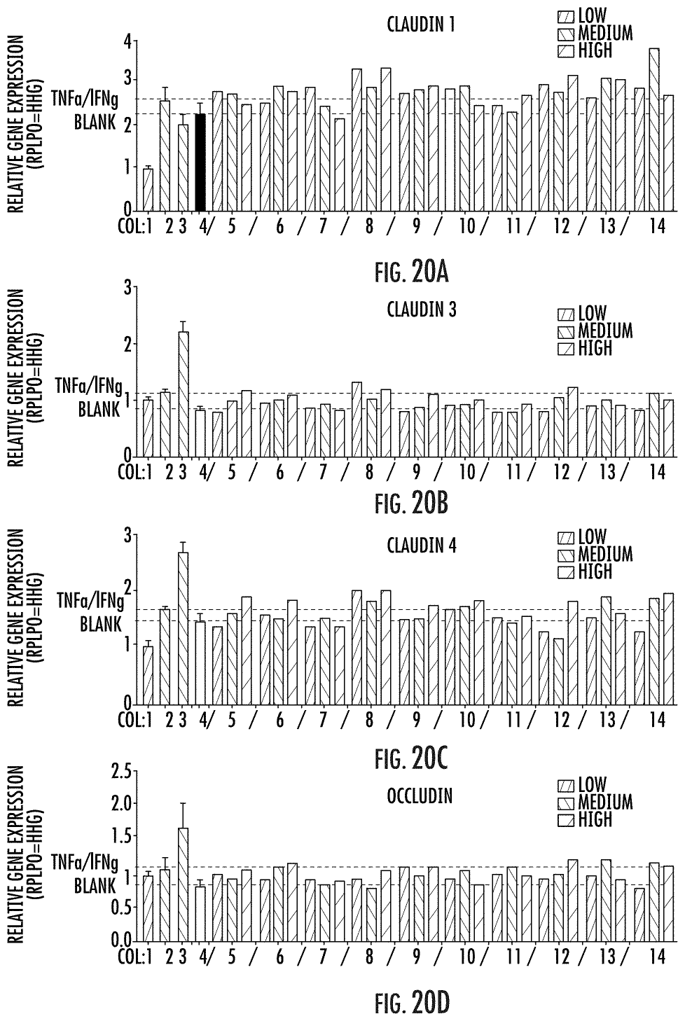

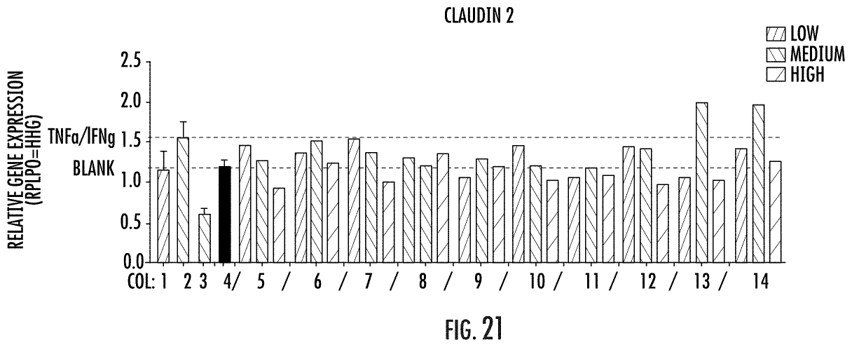

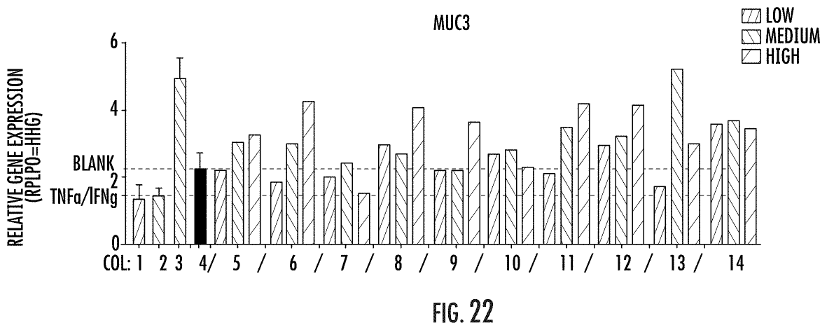

Apical treatment of the cells with the positive control NaB increased the expression of claudin-3, claudin-4 and occludin (i.e. tighter junctions) (FIG. 20B through 20D); while it decreased the expression of claudin-2 (i.e., decreasing permeability—a favorable outcome for barrier function) (FIG. 21), compared to the TNFα/IFNγ control. No effect of NaB was seen on the expression of claudin-1 (FIG. 20A). Finally, NaB strongly increased the expression of mucin-3 (FIG. 22). The positive control NaB behaved as expected by strengthening tight junctions and Increasing mucin-3 production.

In general, the effects of the treatment colonic suspensions on the expression of the barrier-strengthening tight junction genes were minor (FIG. 16). This is in contrast with Example 3, which showed Increased protein levels. Furthermore, the effects were mostly restricted to claudin-4 expression. At the highest concentration, peptide blend, L-theanine and the combination of trace mineral complex+L-theanine+peptides tended to increase the expression of claudin-4 compared to both the TNFα/IFNγ and blank control. In addition, XOS prebiotic fiber tended to increase claudin-4 expression at all concentrations and the combination of L-theanine+peptides at its medium concentration. The inulin control tended to increase the claudin-4 expression at the two highest concentrations. Regarding claudin-1 expression, XOS prebiotic fiber (lowest and highest concentration) and the medium concentration of the inulin control tended to slightly increase the expression of claudin-1 compared to the TNFα/IFNγ and blank control. None of the treatments increased the expression of claudin-3 and occludin compared to the TNFα/IFNγ control. However, when compared to the blank control, a slight increase in claudin-3 and/or occludin expression could be observed. Indeed, claudin-3 expression dose-dependently increased upon administration of peptide blend, L-theanine and the combination of trace mineral complex+L-theanine+peptides. Furthermore, XOS prebiotic fiber increased claudin-3 expression at all concentrations; while nucleotides only at the highest concentration. The two highest concentrations of the inulin control also increased the claudin-3 expression. Concerning the occludin expression, L-theanine and the combination of trace mineral complex+L-theanine+peptides increased occludin expression in a dose-dependent manner, compared to the blank control. Also, the highest concentration of peptide blend and XOS prebiotic fiber, all concentrations of nucleotides and the full product and the two lowest concentrations of polyphenol prebiotic blend and the combination of L-theanine+peptides did increase the expression of occludin compared to the blank control. Finally, also the two highest concentrations of inulin Increased the occludin expression.

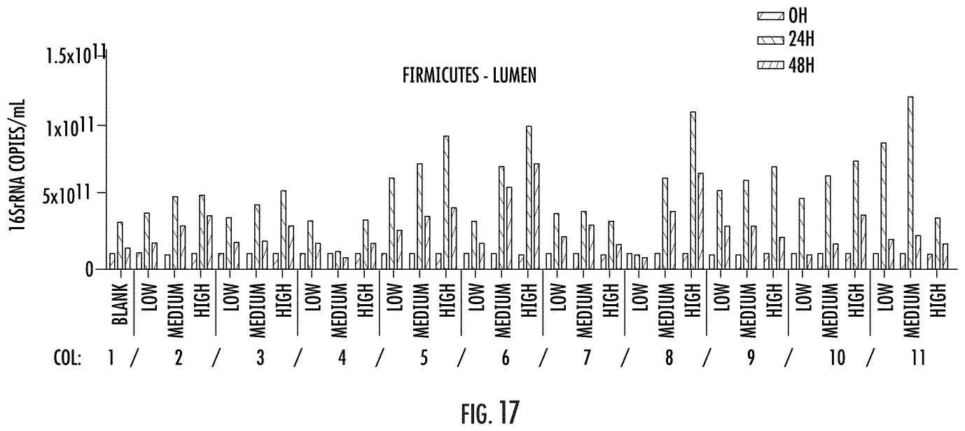

In contrast to the effects on the tightening genes, the effects of the treatment colonic suspensions on the expression of the permeability-inducing claudin-2 gene were more pronounced (FIG. 17). Most of the colonic batch samples, including the blank control, decreased the expression of claudin-2 compared to the TNFα/IFNγ control. In addition, at the highest concentration, peptide blend, trace mineral complex, polyphenol prebiotic blend, the full product, the combination of trace mineral complex+L-theanine+peptides and of L-theanine+peptides further decreased the expression of claudin-2 compared to the blank control. The medium concentration of L-theanine+peptides and of the inulin control increased the expression of claudin-2.

All colonic batch samples, including the blank control, increased the expression of mucin-3 compared to the TNFα/IFNγ control (FIG. 18). In addition, the combination of trace mineral complex+L-theanine+peptides and the inulin control increased the expression of mucin-3 compared to the blank control at all concentrations tested. Furthermore, the two highest concentrations of peptide blend, L-theanine, the full product and the combination of L-theanine+peptides further increased the expression of mucin-3 compared to the blank control. Finally, the highest concentration of nucleotides and the lowest and highest concentration of XOS prebiotic also further increased the expression of mucin-3. In general, the full product and all subfractions, except for trace mineral complex and polyphenol prebiotic blend, increased the expression of mucin-3 at least at the highest concentration tested.

The full product and its subfractions in this experiment on the expression of the barrier-strengthening tight junction genes all increased claudin-4 expression. The effects on the permeability-inducing claudin-2 gene were more pronounced; with decreased reported expression upon treatment with the highest concentration of peptide blend, trace mineral complex, polyphenol prebiotic blend, the full product, the combination of trace mineral complex+L-theanine+peptides and the combination of L-theanine+peptides. Finally, the full product and all subfractions, except for the trace mineral complex and polyphenol prebiotic blend, increased the expression of mucin-3 at least at the highest concentration tested, with the full product being active in increasing mucin-3 expression at both the medium and high concentrations.

There appears to be some conflict between the tight junction protein expression (actually qPCR) of Example 2 and the protein level measurements of Example 3. One would expect the results to be more consistent. Example 3 clearly showed increased tight junction protein levels as measured by antibody detection. So there must have been increased expression. The Example 3 results were generated in a controlled system (no donor dependence, unlike Example 2) and they are consistent with one another.

### Example 3: In Vitro Assessment of Effect on Intestinal Surface and Barrier Function

The following test substance preparations containing each Ingredient in amounts outlined below were evaluated in this experiment. The combination products contain each ingredient in the same proportions as in Example 2.
Peptide blend
L-theanine
trace mineral complex
XOS prebiotic fiber
Nucleotides
Polyphenol prebiotic blend
Combination of 2 components (L-theanine and trace mineral complex)
Combination of 3 components (L-theanine, trace mineral complex and plant peptide blend)
Combination of all components (L-theanine, trace mineral complex, plant peptide blend, XOS prebiotic, polyphenol blend and nucleotides), referred to as “full product”
Control (no treatment, media only)
Inulin was used as a positive control.

Microvilli proteins that can serve as markers which when upregulated Indicate an increase in microvilli construction include the following:

Villin (George et al. 2007) a marker for microvilli of the brush border • myosin (Klooster et al. 2009) marker for the microvilli of the brush border and microvilli induction • MLPCDH aka cadherin (Crawley et al. 2014b) a marker for microvilli of the brush border • Sucrase-isomaltase (Tyska und Mooseker 2004) a marker for enterocytes of the brush border. See, George, Michael D.; Wehkamp, Jan; Kays, Robert J.; Leutenegger, Christian M.; Sabir, Sadiah; Grishina, Irina et al. (2008): In vivo gene expression profiling of human intestinal epithelial cells: analysis by laser microdissection of formalin fixed tissues. In: BMC genomics 9, S. 209. DOI: 10.1186/1471-2164-9-209. George, Sudeep P.; Wang, Yaohong; Mathew, Sijo; Srinivasan, Kamalakkannan; Khurana, Seema (2007): Dimerization and actin-bundling properties of villin and its role in the assembly of epithelial cell brush borders. In: J. Biol Chem. 282 (36), S. 26528-26541. DOI: 10.1074/jbc.M703617200. Klooster, Jean Paul ten; Jansen, Marnix; Yuan, Jin; Oorschot, Viola; Begthel, Harry; Di Giacomo, Valeria et al. (2009): Mst4 and myosin induce brush borders downstream of the Lkb1/Strad/Mo25 polarization complex. In: Developmental cell 16 (4), S. 551-562. DOI: 10.1016/j.devcel.2009.01.016. Crawley, Scott W.; Shifrin, David A.; Grega-Larson, Nathan E.; McConnell, Russell E.; Benesh, Andrew E.; Mao, Sull et al. (2014b): Intestinal brush border assembly driven by protocadherin-based intermicrovillar adhesion. In: Cell 157 (2), S. 433-446. DOI: 10.1016/j.cell.2014.01.067. Tyska, Matthew J.; Mooseker, Mark S. (2004): A role for myosin-1A In the localization of a brush border disaccharidase. In: J Cell Biol 165 (3), S. 395-405. DOI: 10.1083/jcb.200310031.

Barrier function can be assessed by various tight junction proteins and/or TEER:
Occludin
Claudin-1
Zona occludens.
France M M, Turner J R. The mucosal barrier at a glance. J Cell Sci. 2017; 130(2):307-314. doi:10.1242/jps.193482
Günzel D, Yu A S. Claudins and the modulation of tight junction permeability. Physiol Rev. 2013; 93(2):525-560. doi:10.1152/physrev.00019.2012
Khan N, Asif A R. Transcriptional regulators of claudins in epithelial tight junctions. Mediators Inflamm. 2015:2015:219843. doi:10.1155120151219843
Luettig J, Rosenthal R, Barmeyer C, Schulzke J D, Claudin-2 as a mediator of leaky gut barrier during intestinal inflammation. Tissue Barriers. 2015; 3(1-2):e977176. Published 2015 Apr. 3. doi:10.4161121688370.2014.977176 Zuo L, Kuo W T, Turner J R. Tight Junctions as Targets and Effectors of Mucosal Immune Homeostasis. Cell Mod Gastroenterol Hepatol. 2020:10(2):327-340. doi:10.1016/j.jcmgh.2020.04.001

The CaCo-2 model was utilized to show the ability of the test formulations to positively influence the intestinal surface by measuring molecular markers known to be indicative of and/or correlate with intestinal surface modulation and barrier function. The CaCo-2 model is a well-known model for the evaluation of processes in the human intestine. The cells are cultured as a monolayer and are induced to differentiate and to express enterocyte properties.

One main determinant of the intestinal surface are microvilli formed on enterocytes, in particular, the number of microvilli on each enterocyte. These structures increase the surface of the cell thus providing a better contact to the luminal content. Microvilli are generated by the cell and comprise structural proteins like Actin, Villin, Espin, and Fimbrin. More recently other proteins Involved in the formation of microvilli have been described, e.g., tyrosine kinase substrate EP58, Ezrin, Radixin, Harmonin, Moesin or myosins, for example, Myosin-la amongst them. Other markers are representative of enterocytes as such, e.g., alkaline phosphatase or sucrase-isomaltase.

In parallel to markers of microvilli, markers of intestinal barrier like claudins, occludin, Zona occludens 1 (ZO-1) or trans epithelial resistance were analyzed. The intestinal barrier function needs to be tightly regulated in order to maintain a well-balanced status enabling the uptake of nutrients from the intestinal lumen while hindering the entrance of harmful microorganisms into the body tissue. Conditions which negatively influence the viability of intestinal epithelial cells naturally cause a loss of barrier integrity. In other words, toxicity and barrier function are directly linked.

Selected markers (Villin, Sucrase-isomaltase, Cadherin, Myosin, Occludin, Claudin1 and Zona occludens 1) were measured to demonstrate the effects of a treatment on microvilli generation in vitro and differences in microvilli amount or barrier function between treatment groups.

There are two main methods to analyze these markers/proteins of interest, namely, quantitative RT-PCR and ELISA. RT-PCR is used to measure the expression or mRNA coding the marker proteins noted above. ELISA is used to detect the expressed protein itself. ELISA usually overcomes the additional influence of post translational regulation processes. In this experiment, ELISA was used but the option to utilize RT-PCR was preserved.

Except if otherwise indicated, all 10 test products listed above were used in the present experiments. Maximal applicable dosages of the respective compositions under test were determined for CaCo-2 cells. CaCo-2 cells (ATCC HTB-37TM) were cultured on 96-well plates (100 μl/well) and were treated for 12 days with seven concentrations of each test mixture, including a change of media every 2-3 days. Toxic positive controls and negative controls (media only) were tested in parallel. After treatment, the number of living cells in each treatment group was determined by measuring the transformation of a vital dye (methyl tetrazolium salt (MTT)) to its metabolite (formazan) at wavelengths of 590 nm against 750 nm on Tecan Infinite M200pro instrument (Tecan, Austria). Applicable concentrations should not reduce the number of viable cells to less than 70% of the untreated control.

After the evaluation of applicable concentrations of the test mixtures, CaCo-2 cells were cultured in Minimum Essential Medium (Blochrom, Berlin GERMANY) with 20% FBS, 1% non-essential amino acids, 1% penicillin/Streptomycin, with an appropriate concentration of test products for a defined time, for example, 6 days or 12 days. The cultures were analyzed for the presence and relative quantity of the markers of the intestinal surface. Treated cultures were compared to the control (medium only or untreated) to show differences that support increased numbers of microvilli and/or enterocytes. The results were normalized to the respective protein content of the culture as reference for differences in cell number of the cultures.

A set up based on CaCo-2 models including negative control, positive control (if used) and eight test mixtures was used to perform experiments in two independent runs evaluating, for example, four markers of the intestinal surface.

a) Villin, a marker for microvilli of the brush border;
b) Myosin, a marker for the microvilli of the brush border and microvilli induction;
c) MLPCDH/MUPCDH=Cadherin, a marker for microvilli of the brush border; and
d) Sucrase-isomaltase, a marker for enterocytes of the brush border.

Evaluation of the Barrier Function:

Markers of the barrier function based of the trans-epithelial electrical resistance were determined. Barrier function was also measured by ELISA based on the analysis of the following markers of tight junctions:
a) Claudin 1
b) Occludin
c) Zona occludens (ZO-1)

The test products were dissolved either in cell culture medium, using ethanol or DMSO as solvent as follows.

TABLE 15
Stock solutions of test products

| | Solution found [mg | |
|---|---|---|
| | powder/mL solvent] | Solvent |
| Peptide blend | 20 | DMSO |
| L-Theanine | 200 | medium |
| L-Theanine/peptide blend | 20 | medium |
| Trace mineral complex + L- | 20 | medium |
| Theanine + peptides | | |
| Trace mineral complex | 2 | DMSO |
| XOS | 200 | medium |
| inulin | 2 | medium |
| Polyphenol prebiotic | 2 | medium |
| Full product | 20 | DMSO |

The stock solutions listed the foregoing table were applied as starting doses for the subsequent dose-determining experiments. However, DMSO needed to be diluted prior to the application to the cells at minimum 1:100(1%) since higher concentrations of DMSO are known to be cytotoxic.

TABLE 16
Dose-determining experiment. Doses applied.

| | Stock solution | | | | | | | | |
|---|---|---|---|---|---|---|---|---|---|
| | [mg powder/ | Concentration [mg powder/mL] | | | | | | | |
| | mL solvent] | 1 | 2 | 3 | 4 | 5 | 6 | 7 | 8 |
| Peptide blend | 20 | 0.2 | 0.075 | 0.027 | 0.01 | 0.0039 | 0.0014 | 0.0005 | 0.0002 |
| L-Theanine | 200 | 200 | 74.55 | 27.79 | 10.36 | 3.86 | 1.44 | 0.54 | 0.2 |
| L-Theanine + | 20 | 20 | 7.45 | 2.77 | 1.04 | 0.38 | 0.14 | 0.054 | 0.02 |
| peptides | | | | | | | | | |
| Trace mineral | 20 | 20 | 7.45 | 2.77 | 1.04 | 0.38 | 0.14 | 0.054 | 0.02 |
| complex + L- | | | | | | | | | |
| theanine + peptides | | | | | | | | | |
| Trace mineral | 2 | 0.02 | 0.0074 | 0.0027 | 0.001 | 0.0003 | 0.0001 | 0.00005 | 0.00002 |
| complex | | | | | | | | | |
| XOS prebiotic | 200 | 200 | 74.55 | 27.79 | 10.36 | 3.86 | 1.44 | 0.54 | 0.2 |
| Inulin (positive | 2 | 2 | 0.74 | 0.27 | 0.1 | 0.038 | 0.014 | 0.005 | 0.002 |
| control) | | | | | | | | | |
| Polyphenol blend | 2 | 2 | 0.74 | 0.27 | 0.1 | 0.038 | 0.014 | 0.005 | 0.002 |
| prebiotic | | | | | | | | | |
| Full product | 20 | 0.2 | 0.074 | 0.027 | 0.010 | 0.003 | 0.001 | 0.0005 | 0.0002 |

Cells were treated with the doses listed in Table 16 for 12 days. Then the relative amount of living cells was determined by application of a vital dye (MTT) to the cultures. Doses that result in more than 70% viable cells (compared to the medium control) qualify for the subsequent experiments. If possible, a concentration was chosen showing some effect by the treatment (75%-90% viability) to be sure to apply a dose sufficiently high to cause an effect without being cytotoxic. On the other hand, some test products did not cause a reduction of viability even at the maximum concentration that was achievable while limited by the maximum amount dissolvable of the respective test product.

Table 17 lists the results of the dose finding experiments and consequently the doses applied in the main trial on epithelial and barrier function markers.

TABLE 17
Results of the dose finding experiments

| | Concentration chosen |
|---|---|
| | for treatment [mg |
| Sample | powder/mL] |
| Peptide Blend | 0.1 |
| L-theanine | 5 |
| L-theanine + Peptides | 5 |
| Trace mineral complex + L-theanine + peptides | 4 |
| Trace mineral complex | 0.02 |
| XOS prebiotic fiber | 15 |
| Nucleotides 60% from S. cerevisiae) | 2 |
| Polyphenol prebiotic blend | 0.04 |
| Full product | 0.1 |

For the main trial on the influence of the test products on epithelial surface markers or barrier function, the cells were treated for 6 days or for 12 days with the concentrations of compositions listed in Table 17. Within this period, test medium was renewed every two to three days. Additionally, and for comparison, cultures were treated with the culture media only as a control. At the end of the treatment, viability of the cultures was checked by measuring the electrical resistance of the cultures (TEER). The cells were harvested and the protein and RNA was extracted. Two replicate cultures were combined. After measuring the protein content of each sample the samples were frozen and stored until being analyzed by ELISA.

Results:

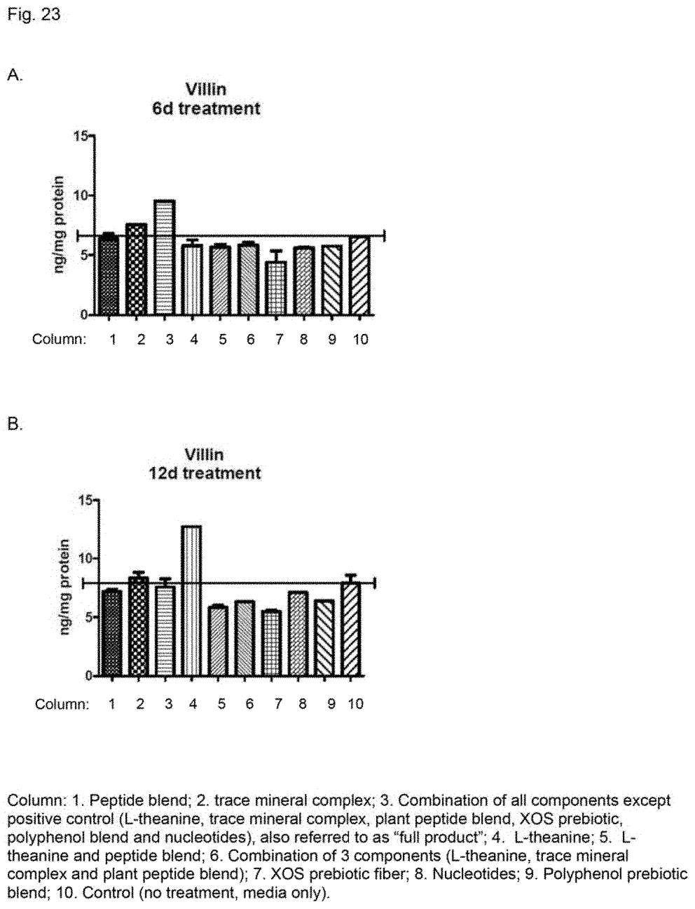

FIG. 23 represents the results of the Villin expression (a marker of the intestinal surface) in CaCo-2 cells after 6 day treatment with a test products or cell culture medium only. The means of two independent experiments are shown. The results are in ng Villin/mg protein+SEM. The horizontal line marks the level of the untreated control (median only).

It is believed that the 6-day data are overall more relevant than the 12-day data given the testing system. The gut is the fastest renewing organ in the body. The small intestine turns over every 3-5 days, and the colon every 5-7 days. The inner mucus layer, depending upon the mucosal environment, about every hour. Groos, S., et al., Changes in epithelial cell turnover and extracellular matrix in human small Intestine after TPN. The Journal of surgical research, 2003. 109: p. 74-85. Johansson, M. E., Fast renewal of the distal colonic mucus layers by the surface goblet cells as measured by in vivo labeling of mucin glycoproteins. PLoS One, 2012. 7(7): p. e41009.

This makes the 6-day data the most relevant. To get growing Caco cells to the point of differentiation as in the intestinal brush border usually requires 2 weeks, but the present experiments started once these cells had reached differentiation. Many protocols other than the one used here evaluate test materials are evaluated within shorter time frames. The present experiments were permitted to extend to 12 days in an exploratory vein to continue to monitor mechanism and activity. The time interval 6 to 12 days is not intended to be physiologically representative of the normal time food spends in the small intestine or even the cellular lifespan of enterocytes or the half-life of the mucin that covers them.

Trace mineral complex and the full product stimulated Villin expression to a higher extent than the untreated control. Trace mineral complex outperformed every group at 6 days except the full product. It did significantly better alone than when combined with L-theanine and peptides (the effect does not translate from the ingredient alone to the combination most likely because of dilution) and it failed to exponentially increase the effect of the full product (also because it is diluted), but the performance of the trace mineral complex alone indicates that the dose in the combinations in all experiments herein should have been increased (the combinations use the same proportions of ingredients as in the full product).

L-theanine, used alone, did not perform well at 6 days in stimulating villin or myosin at 6 days although the effect was consistently strong at 12 days.

The XOS test product may reduce the expression of Villin. All other test products did not stimulate or reduce Villin expression in comparison to the untreated control.

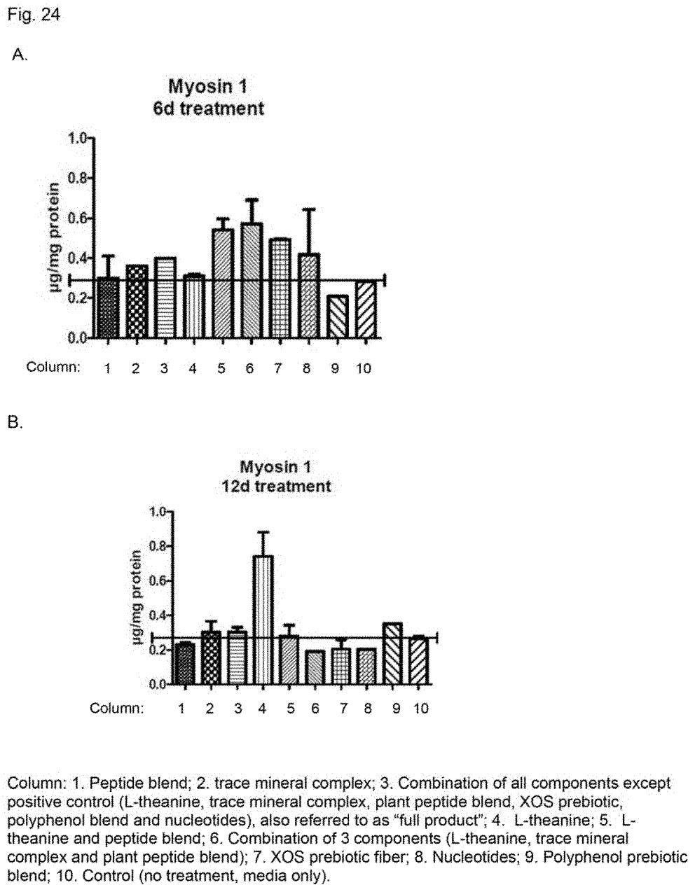

FIG. 24 shows Myosin 1 expression in CaCo-2 cells after treatment with the test products or cell culture medium only control. Means of two Independent experiments are shown. Results are in ng Myosin/mg protein+SEM. The horizontal line marks the level of the cell culture medium only control.

The stimulation of Myosin 1 expression by test products is shown in FIG. 24. After 6 days of treatment with the test products, six of the test products, (Trace mineral complex, full product, L-Theanine/peptide combination, Trace mineral complex/L-Theanine/peptide combination, XOS and inulin) stimulated Myosin 1 expression to a higher extent than the control. The polyphenol prebiotic test product reduced the expression of Myosin 1.

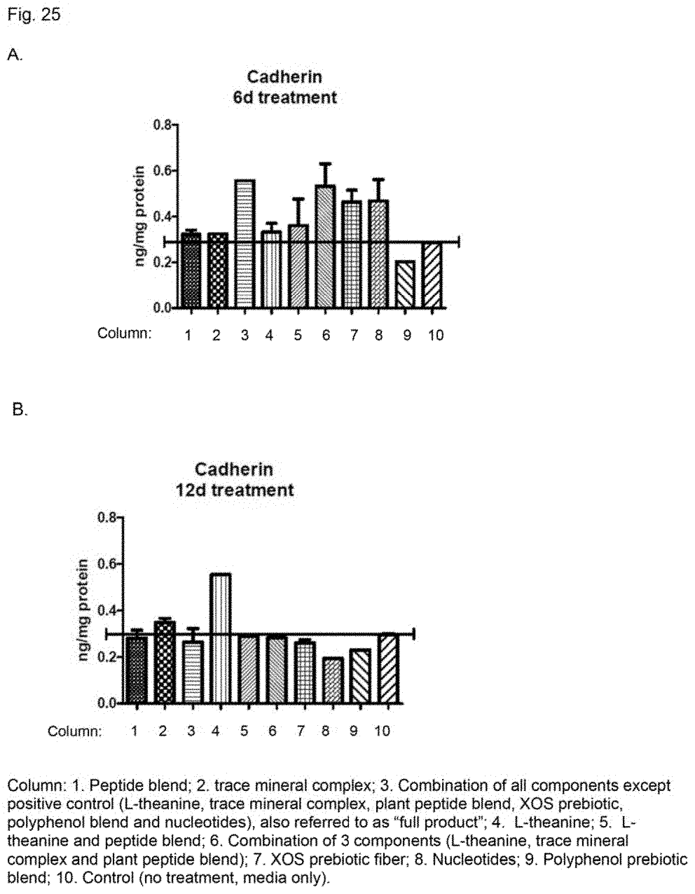

FIG. 25 shows Cadherin expression in CaCo-2 cells after treatment with the test products or cell culture medium only control. Means of two independent experiments are shown. Results are in ng Cadherin/mg protein+SEM. The horizontal line marks the level of the cell culture medium only control.

After 6 days of treatment with the test products, full product, L-theanine/peptide combination, Trace mineral complex/L-theanine/peptide combination, XOS and inulin stimulated Cadherin expression to a higher extent than the control. The polyphenol prebiotic reduced the expression of Cadherin.

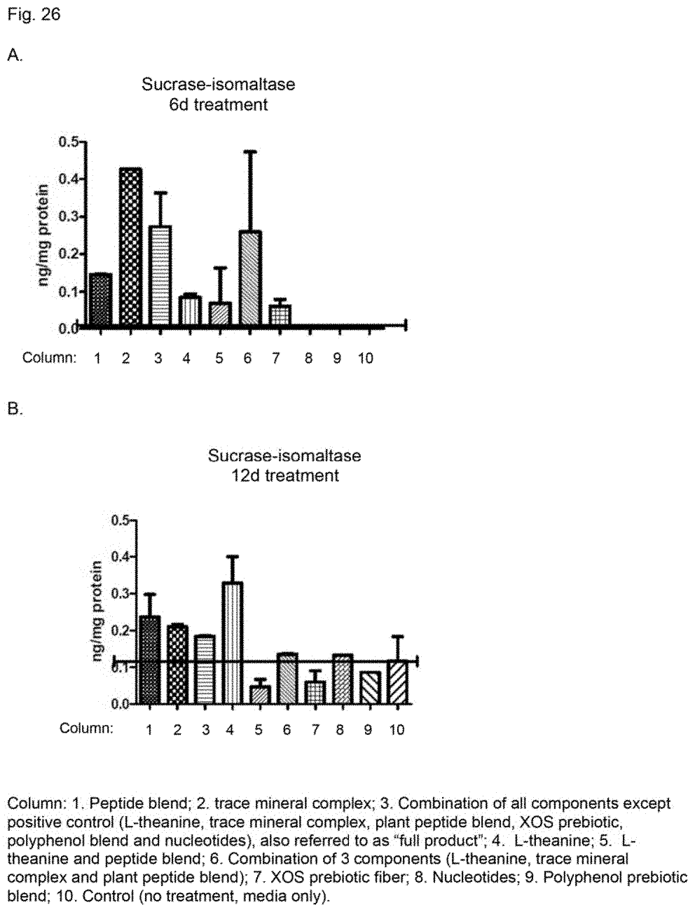

FIG. 26 shows Sucrase-isomaltase (SI) expression in CaCo-2 cells after treatment with the test products or cell culture medium only control. Means of two independent experiments are shown. Results are in ng SI/mg protein+SEM. The horizontal line marks the level of the negative (medium only) control.

After 6 days of treatment, the SI value of the negative control was almost undetectable. With the exception of inulin and the polyphenols, all treatments induced SI. Trace mineral complex, full product and trace mineral complex/L-theanine/peptide combination Induced SI.

Taken together, markers of intestinal surface were heterogeneously stimulated by different test products after 6 days of treatment. The full product consistently induced markers of intestinal surface.

Furthermore, one of the most active inducers of the intestinal surface was the combination of trace mineral complex/L-theanine/peptide combination. XOS test product and the positive control inulin were more selective in inducing Myosin 1 and Cadherin.

Polyphenols reduced the majority of the intestinal surface markers after 6 days of treatment.

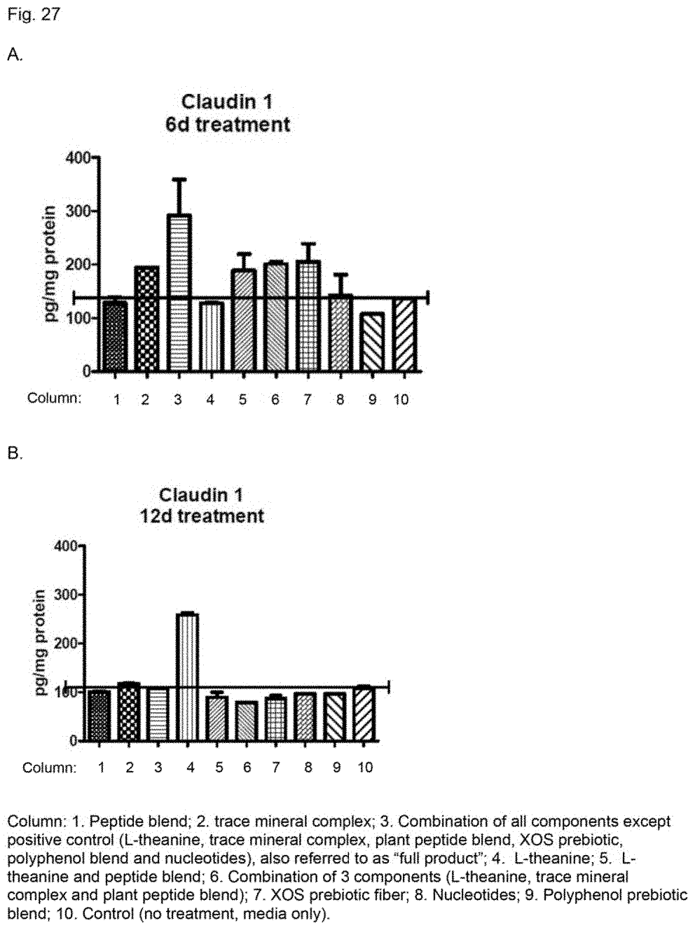

Claudin 1 is used as a marker of intestinal barrier function. FIG. 27 shows the results of Claudin 1 expression in CaCo-2 cells after treatment with the test products or negative (medium only) control.

Means of two independent experiments are shown. Results are in pg Claudin/mg protein+SEM. The horizontal line marks the level of the negative control.

As shown in FIG. 27, after 6 days of treatment the full product induced high-level expression of Claudin 1. Trace mineral complex, L-theanine/peptide combination, trace mineral complex/L-theanine/peptide combination and XOS also induced the expression of Claudin 1.

Polyphenols Reduced the Expression of Claudin 1.

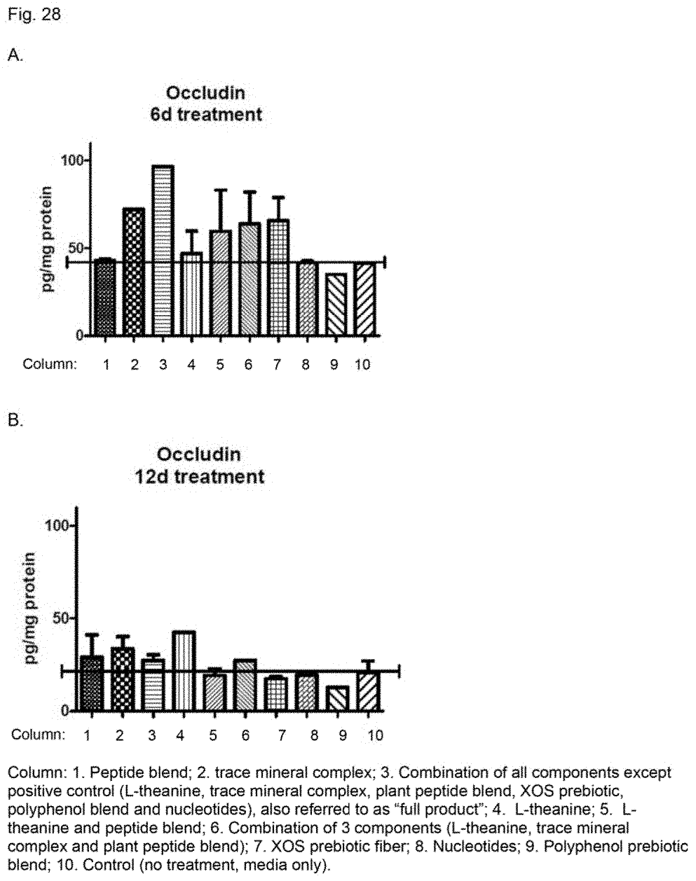

Occludin is used as a marker of intestinal barrier function. FIG. 28 shows the results of Occludin expression in CaCo-2 cells after treatment with the test products or cell culture medium only control. Means of two independent experiments are shown. Results are in pg of Occludin/mg protein+SEM. The horizontal line marks the level of the negative control.

As shown in FIG. 28 after 6 days of treatment, the full product complete formula induced high-level expression of Occludin. Trace mineral complex, L-Theanine/peptide combination, Trace mineral complex/L-Theanine/peptide combination and XOS also induced the expression of Occludin.

Zona occludens 1 (ZO-1) protein expression is used as a marker of intestinal barrier function. FIG. 7X shows the results of ZO-1 expression in CaCo-2 cells after treatment with the test products or negative control. Means of two independent experiments are shown. Results are in pg ZO-1/mg protein+SEM.

The horizontal line marks the level of the negative (culture medium only) control.

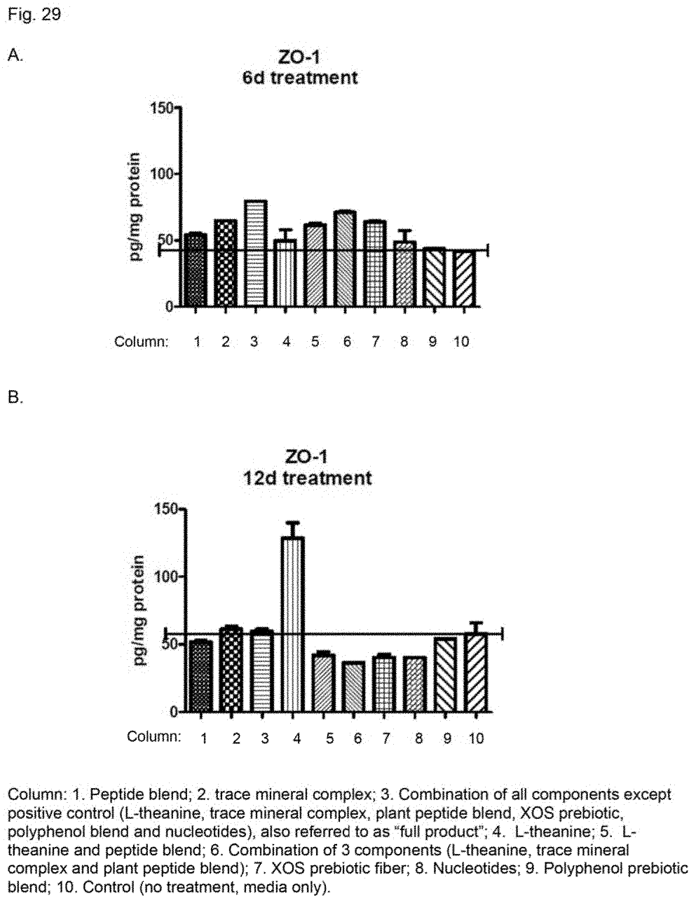

As shown in FIG. 29, after 6 days of treatment ZO-1 protein expression was induced by every treatment formulation in comparison to control, with the full product demonstrating a higher level of ZO-1 protein expression as compared to other products tested.

It may be concluded from the foregoing that the both the trace mineral complex and the full product induced all protein markers tested in this experiment (Villin, Myosin, Cadherin and Sucrase-isomaltase) which are markers of the intestinal surface; the full product was the most consistent performer across all of the data points. The data indicate that the full product exhibits a positive effect on both intestinal surface and barrier function. The data Indicate that the full product is likely to benefit from Inclusion of higher amounts of trace mineral complex as that ingredient was present in the full product in extremely low amounts. Even the lowest dose of the trace mineral complex, tested alone, contained more of this ingredient than the highest dosage of the full product. The combination product containing trace mineral complex falls in-between. Based on the results of the present experiments as well as on those of the prior examples, the lowest limit of the range of trace mineral complex effective amounts for most purposes discussed in this disclosure can be set at 50 and preferably 85 grams daily, as set forth elsewhere herein. Both the trace mineral complex and the full product induced the protein markers tested in this experiment that are associated with improved barrier function of the intestinal epithelium (claudin 1, occludin and ZO-1).

### Example 4: In Vivo Mouse Study

The effects of treatment with test combination products and test components are evaluated in mice with respect to, more favorable gut structure (increased mucin formation, Increased mucin turnover, increased or optimized gut surface area), and more effective barrier function as these contribute to maintaining a healthy balance in the lumen environment, a favorable microbiome ecology and efficient energy metabolism in the host. A higher minimum amount of trace mineral complex is employed, corresponding to a human daily dose of 50 mg or preferably 85 mg in the full product and all combinations.

A method of measuring murine gut surface area is described in Casteleyn, C. et al, Laboratory Animals 2010; 44: 176-183. DOI:10.1258/la.2009.009112. The following fractions are administered daily to mice: negative control (no administration), L-theanine component, bioactive peptide component, polyphenol component, XOS nondigestible carbohydrate fraction, mineral complex fraction, or combination of three components (bioactive peptides, L-theanine and mineral complex) and a combination of all of the foregoing (full product). The proportions of the individual components in the combination products are the same but the amount of trace mineral complex is increased based on the results of Examples 2 and 3.

The following parameters are evaluated in the treated mice:
Intestinal barrier function (one or more of mucin amount, mucin turnover).
Tight junction transcriptome and production of Ti proteins (mRNA) as per Example 2 or 3
Microvilli (height to crypt depth ratio)
inflammatory cytokines and markers (IL-1beta, TNF-alpha, INF-gamma)
Metabolic products produced by bacteria, especially SCFA and especially butyrate
Host-microbiome interactions
Surface area (fully stretched intestinal length, which would be influenced in large measure by the intestinal folds or villi).

The dietary supplements of the present disclosure and methods of their use have been described above by reference to particular embodiments. It will be apparent to those skilled in the art however in light of the present disclosure that many variations are possible all within the scope of the present disclosure. In particular, the present Examples contain general disclosure not dependent upon the particular compositions used in the experiments described in these sections. Such general discussion should therefore be recognized as such and not be tethered to the specific experimental system and test products and methods described in the Examples.

The disclosure of cited references are hereby incorporated by reference in their entirety for all purposes.

1-43. (canceled)

44. A human dietary supplement comprising a mixture of L-theanine and a trace mineral complex, and at least one or at least two bioactive peptides, wherein the amount of L-theanine is in a daily dose within the range from about 25 mg to about 1,000 mg, the amount of trace mineral complex is in a daily dose from about 5 mg to about 1,000 mg, and the total amount of bioactive peptides is in a daily dose from about 50 mg to about 10 g wherein the dietary supplement promotes gut health.

45. The supplement of claim 44 wherein an improvement in gut health is assessed in vivo or in vitro by improvements in at least two of the following parameters: mucous membrane integrity; increase in one or more tight junction protein expression (or a decrease in claudin-2); increase in mucin production; increase in mucin turnover: increase in microvilli-related gene transcription and/or expression; decrease in inflammatory markers and/or cytokines; increase in gut surface area; increase in short chain fatty acid production; reduction in pH; increase in lactate and/or gas production; and a favorable gut microbiota profile shift.

46. The supplement of claim 44 wherein an improvement in gut health comprises strengthening, maintenance and/or repair of the intestinal barrier and/or maintenance and/or restoration of luminal balance.

47. The supplement according to claim 44 further comprising: (i) one or more prebiotics selected from the group consisting of nondigestible fiber oligosaccharides; (i) one or more polyphenol prebiotics; and (iii) one or more phage prebiotic.

48. The supplement according to claim 47 wherein the one or more nondigestible carbohydrate prebiotics are provided in an amount from about 300 mg to about 5 g if the nondigestible carbohydrate prebiotic comprises XOS, or from about 1,000 mg to about 15 g if the nondigestible carbohydrate prebiotic comprises FOS, IOS or GOS.

49. The supplement according to claim 47 wherein the one or more polyphenol prebiotics is in a daily dose within the range generally from about 10 mg to about 2 g or typically within the range from about 100 mg to about 2 g, or more specifically within the range of about 175 mg to about 1.5 g.

50. The supplement according to claim 47 wherein the one or more phage prebiotic is in a daily dose within the range generally from about 2 mg to about 200 mg, or typically from about 5 to about 100 mg, or more specifically from about 10 to about 50 mg.

51. The supplement according to claim 47 further comprising a daily dose within the range generally from about 45 mg to about 1.8 g, or typically within the range from about 60 mg to 600 g, or more specifically within the range from about 120 mg to 300 mg of a nucleotide mixture prebiotic.

52. The supplement according to claim 47 further comprising an activated enzyme mixture comprising an amylase, a protease, a cellulase and a lipase and optionally further comprising one or more of a galactosidase, an invertase, a maltase, bromelain and papain.

53. The supplement according to claim 44, wherein the peptides comprise at least one or preferably at least two of the following: pea peptide, mung bean peptide, Momordica charantia (bitter melon) peptide, rice peptide, algae peptide, fava bean peptide, spinach peptide, almond peptide, walnut peptide, oyster peptide, algae peptides, wheat peptide, collagen peptide, croix peptide, potato peptide, salmon peptide, corn peptide, sea cucumber peptide, yeast peptide, egg peptide, albumin peptide, milk peptide, casein peptide, amaranth peptide, silk peptide, soy peptide, oat peptide, quinoa peptide, and pulses peptides.

54. The supplement according to claim 44 wherein the trace mineral complex is selected from the group consisting of a composition comprising 74 trace minerals including boron, cobalt, copper, iron, manganese, vanadium and zinc, the composition derived from rhyolitic tuff breccia also comprising volcanic glass and clay.

55. The supplement according to claim 50 wherein the at least one phage prebiotic comprises myoviridae and siphoviridae.

56. The supplement according to claim 44 wherein the supplement is formulated in one or more daily dosages for at least 30 days.

57. The supplement of claim 44 wherein the mineral complex is derived from a rhyolitic tuff breccia.

58. The supplement of claim 44 wherein the daily dose is achieved by a plurality of dosage forms of the same or different composition.

59. A method for promoting gut health comprising administering to a subject a dietary supplement according to claim 44.

60. The method of claim 59 wherein promoting gut health comprises an improvement in at least two of the following an increase in membrane integrity; an increase in tight junction protein expression: an increase in mucin production: an increase in mucin turnover; an increase in microvilli-related gene transcription/expression; a decrease in inflammatory markers and/or cytokines; an increase in gut surface area; an increase in short chain fatty acid production; a decrease in pH; an increase in lactate and gas production; and a gut microbiota profile shift.

61. The method of claim 59 wherein promoting gut health comprises strengthening, maintaining and/or repairing the intestinal barrier and maintaining and/or restoring luminal balance.

62. The method of claim 59 wherein the administration continues for at least 30 days.

63. The supplement of claim 44 wherein the amount or amounts is/are effective to accomplish one or more of the following:
increase the amount of total short-chain fatty acid production or of at least one of propionate, acetate and butyrate;
increase the expression or level of at least one tight junction protein (for example, claudin-1, claudin-3, claudin-4, occludin, or a zona occludens protein) or mucin-3 or decrease the expression or level of claudin-2;
increase transepithelial electrical resistance:
increase the abundance or diversity of a member or phylum of the intestinal bacterial community of the intestinal lumen or mucus area (for example, Firmicutes, Bacteroidetes, Lactobacilli, Bifidobacteria, F. prausnitzii, or A. muciniphila);
increase the expression or level of at least one intestinal surface area marker protein (for example, villin, myosin, cadherin or sucrase isomaltase).

64. The supplement of claim 44 wherein the amount of trace mineral complex is in a daily dose within the range of 50 to 1,000 mg and preferably 85 to 1000 mg; the amount of L-theanine is in a daily dose within the range from about 25 mg to about 1,000 mg, and the amount of bioactive peptides, when present in the dietary supplement, is in a daily dose from about 50 mg to about 10 g.

65. The supplement of claim 44 further comprising at least one of the following:
one or more and preferably two or more polyphenolic prebiotics in a daily dose within the range generally from about 10 mg to about 2 g or typically within the range from about 100 mg to about 2 g, or more specifically within the range the range of about 50 mg to about 1.5 g;
one or more nondigestible carbohydrate prebiotics in an amount from about 300 mg to about 5 g if the nondigestible carbohydrate prebiotic comprises XOS (which is preferred), or from about 1,000 mg to about 15 g if the nondigestible carbohydrate prebiotic comprises FOS, IOS or GOS; and
one or more nucleotide mixture prebiotic in a daily dose within the range generally from about 45 mg to about 1.8 g, or typically within the range from about 60 mg to 1 g, or more specifically within the range from about 120 mg to 600 mg;
one or more phage prebiotics in an amount within the range from about 2 mg to about 200 mg, or typically from about 5 to about 100 mg, or more specifically from about 10 to about 50 mg.

66. A method for Increasing intestinal surface area and/or improving intestinal barrier function comprising administering to a subject an effective amount for that purpose of a composition comprising a product selected from the group consisting of a trace mineral complex, L-theanine, a combination of (a) L-theanine+bioactive peptides or (b) L-theanine+bioactive peptides+trace mineral complex or (c) L-theanine and trace mineral complex; and a combination of L-theanine+bioactive peptides+trace mineral complex+optionally, one or more of (i) a polyphenol prebiotic, (ii) a nondigestible fiber prebiotic, and (iii) a polynucleotide.

67. The method of claim 66 wherein the increase in surface area is assessed by an increase in one or more proteins of intestinal villi such as one or more of villin, myosin, cadherin and sucrase-isomaltase; and/or intestinal barrier function is assessed by assessing the expression or level of at least one of claudin-1, claudin-2, claudin-3, claudin-4, occludin and a protein of zona occludens (such as ZO-1), and mucin, such as mucin-3, or by a decrease in claudin-2, or by an increase in TEER.

68. A method for increasing the efficiency of fermentation comprising administering to a subject an effective amount for that purpose of a composition comprising one or more of the following: a trace mineral complex, L-theanine, a combination of (a) L-theanine+bioactive peptides or (b) L-theanine+bioactive peptides+trace mineral complex or (c) L-theanine and trace mineral complex; and a combination of L-theanine+bioactive peptides+trace mineral complex+optionally, one or more of (i) a polyphenol prebiotic, (ii) a nondigestible fiber prebiotic, and (iii) a polynucleotide each preferably on a daily basis.

69. The method of claim 68 wherein the efficiency of fermentation as assessed by increased levels of one or more SCFA (acetate, propionate and butyrate) or total SCFA and/or lactate levels or pH decrease or gas production.

70. A method for increasing the abundance or diversity of members of the intestinal bacterial community (including one or more of Bacteroidetes or Firmicutes phyla or one or more of Lactobacilli or Bifidobacteria, or Faecalibacterium prausnitzii) comprising administering to a subject a composition comprising one or more of the following: a trace mineral complex, L-theanine, a combination of (a) L-theanine+bioactive peptides or (b) L-theanine+bioactive peptides+trace mineral complex or (c) L-theanine and trace mineral complex; and a combination of L-theanine+bioactive peptides+trace mineral complex+optionally, one or more of (i) a polyphenol prebiotic, (ii) a nondigestible fiber prebiotic, and (Iii) a polynucleotide, each preferably on a daily basis.

71. A method for increasing abundance and/or diversity of or increasing the efficiency of fermentation by an intestinal microbial community the method comprising administering to a subject an amount effective for that purpose of a composition comprising a polyphenolic preparation containing one I or more and preferably two or more polyphenolic prebiotics.

72. The method of claim 71 wherein the amount is in a daily dose within the range generally from about 10 mg to about 2 g or typically within the range from about 100 mg to about 2 g, or more specifically within the range the range of about 50 mg to about 1.5 g.

73. A method for increasing the abundance or diversity of members of the intestinal bacterial community (including one or more of Bacteroidetes or Firmicutes phyla or one or more of Lactobacilli or Bifidobacteria, or Faecalibacterium prausnitzii) comprising administering to a subject a composition comprising one or more of the following: a combination of (a) L-theanine+at least one or at least two bioactive peptides or (b) L-theanine+at least one or at least two bioactive peptides+trace mineral complex or (c) L-theanine+at least one or at least two bioactive peptides+trace mineral complex+optionally, one or more of (i) a polyphenol prebiotic, (ii) a nondigestible fiber prebiotic, and (iii) a polynucleotide, each preferably on a daily basis.
